(19) United States
(12) Patent Application Publication
(10) Pub. No.: US 2021/0009987 A1
(43) Pub. Date: Jan. 14, 2021

(54) RNA-TARGETING KNOCKDOWN AND REPLACEMENT COMPOSITIONS AND METHODS FOR USE

(71) Applicant: Locana, Inc., San Diego, CA (US)

(72) Inventors: David A. NELLES, San Diego, CA (US); Ranjan BATRA, San Diego, CA (US)

(21) Appl. No.: 16/926,205

(22) Filed: Jul. 10, 2020

Related U.S. Application Data
(60) Provisional application No. 62/872,604, filed on Jul. 10, 2019.
(60) Provisional application No. 62/968,819, filed on Jan. 31, 2020.

(51) Int. Cl. C12N 15/10; C12N 15/861; C07K 14/00

(52) U.S. Cl. CPC C12N 15/102; C07K 14/00; C12N 15/861

(57) ABSTRACT

Disclosed are compositions and methods for specifically targeting and knocking down pathogenic RNA molecules which lead to toxic gain-or-loss-of-function mutations while also replacing the targeted, and knocked down, gene with a therapeutic replacement gene.

### CROSS-REFERENCE TO RELATED APPLICATIONS

This application claims priority to, and the benefit of, U.S. provisional application Nos. 62/872,604, filed Jul. 10, 2019 and 62/968,819 filed Jan. 31, 2020, under 35 USC § 119(e). The contents of each of these applications are hereby incorporated by reference in their entireties.

### FIELD OF THE DISCLOSURE

The disclosure is directed to molecular biology, gene therapy, and compositions and methods for modifying expression and activity of RNA molecules.

### INCORPORATION BY REFERENCE OF SEQUENCE LISTING

The contents of the text file named “LOCN_005_001US_SeqList_ST25”, which was created on Jul. 10, 2020 and is 6.07 MB in size, are hereby incorporated by reference in their entirety.

### BACKGROUND

There has been a long-felt but unmet need in the art for providing effective gain-or loss-of-function gene replacement therapies. There is also a long-felt need in the art for providing effective methods of RNA-targeting systems. The disclosure, thus, provides a combination of RNA-targeting and gene replacement strategies. In particular, the disclosure provides compositions and methods for specifically targeting and knocking down pathogenic RNA molecules, which lead to toxic gain-or-loss-of-function mutations, in a sequence-specific manner while also replacing the targeted, and knocked down, gene with a therapeutic replacement gene.

### SUMMARY

The disclosure provides a composition comprising a nucleic acid sequence encoding an RNA-guided target RNA knockdown and replacement therapeutic comprising (a) an RNA-binding polypeptide or portion thereof, and (b) a therapeutic protein, wherein the RNA-binding polypeptide binds and cleaves a target RNA when guided by a gRNA sequence, wherein a pathogenic RNA comprises the target RNA, and wherein the therapeutic protein is a replacement of gain-or-loss-of-function mutations encoded by the pathogenic RNA.

The disclosure provides a composition comprising a nucleic acid sequence encoding a target RNA knockdown and replacement therapeutic comprising (a) an RNA-binding polypeptide or portion thereof, and (b) a therapeutic protein, wherein the RNA-binding polypeptide binds and cleaves a target RNA or a protein encoded by the target RNA, wherein a pathogenic RNA encoding a pathogenic protein with one or more gain-or-loss-of-function mutations comprises the target RNA, and wherein the therapeutic protein is a replacement protein for the pathogenic protein.

The disclosure also provides a composition comprising a nucleic acid sequence encoding a target RNA knockdown and replacement therapeutic for treating retinitis pigmentosa (RP) comprising (a) an RNA-binding polypeptide or portion thereof; and (b) a therapeutic protein, wherein the RNA-binding polypeptide binds and cleaves a target rhodopsin RNA or a protein encoded by the target rhodopsin RNA, wherein a pathogenic rhodopsin RNA encoding a pathogenic rhodopsin protein with one or more gain-or-loss-of-function rhodopsin mutations comprises the target rhodopsin RNA, and wherein the therapeutic protein is a wild-type rhodopsin protein.

In some embodiments, the RNA-binding polypeptide is a RNA-guided RNA-binding protein. In some embodiments, the RNA-guided RNA-binding protein is Cas13a, Cas13b, Cas13c, or Cas13d. In some embodiments, the RNA-binding polypeptide is a non-guided RNA-binding polypeptide. In some embodiments, the non-guided RNA-binding polypeptide is PUF, or PUMBY protein. In some embodiments, the non-guided RNA-binding polypeptide a PUF or PUMBY fusion protein. In one embodiment, a PUF or PUMBY-based first RNA-binding protein is fused to a second RNA-binding protein which is an zinc-finger endonuclease known as ZC3H12A of SEQ ID NO: 358 (also termed herein E17).

In some embodiments, the therapeutic replacement gene (corresponding disease) is selected from the group consisting of: rhodopsin (Retinitis Pigmentosa), PRPF3 (Retinitis Pigmentosa), PRPF31 (autosomal dominant Retinitis Pigmentosa), GRN (FTD), SOD1 (ALS), PMP22 (Charcot Marie Tooth Disease), PABPN1 (Oculopharangeal Muscular Dystrophy), KCNQ4 (Hearing Loss), CLRN1 (Usher Syndrome), APOE2 (Alzheimer's Disease), APOE4 (Alzheimer's Disease), BEST1 (Eye Disease), MYBPC3 (Familial Cardiomyopathy), TNNT2 (Familial Cardiomyopathy), and TNNI3 (Familial Cardiomyopathy).

In some embodiments, the therapeutic protein is rhodopsin or wild-type rhodopsin. In some embodiments, the therapeutic protein is human rhodopsin. In some embodiments, the therapeutic protein is “hardened” rhodopsin.

In some embodiments of the compositions of the disclosure, the pathogenic rhodopsin RNA comprises or encodes at least one gain-or-loss-of-function mutation.

In some embodiments, the rhodopsin target RNA comprises GCCAGCGTGGCATTCTACATCTTC (SEQ ID NO: 406). In some embodiments, the rhodopsin target RNA comprises CAACGAGTCTTTTGTCATCTACATGT (SEQ ID NO: 462), CGCCAGCGTGGCATTCTACATCTTCA (SEQ ID NO: 463), or CATCTATATCATGATGAACAAGCAGT (SEQ ID NO: 464).

In some embodiments, the target RNA encodes an amino acid sequence comprising ASVAFYIF (SEQ ID NO: 407) at positions 269 to 276. In some embodiments, the target RNA encodes an amino acid comprising YASVAFYIFT (SEQ ID NO: 486) at positions 268 to 277.

In some embodiments, the “hardened” rhodopsin is encoded by a nucleic acid sequence which does not comprise the rhodopsin target RNA comprising GCCAGCGTGGCATTCTACATCTTC (SEQ ID NO: 406).

In some embodiments, the “hardened” rhodopsin is encoded by a nucleic acid sequence comprising GCTTCCGTAGCTTTTTATATTTTT (SEQ ID NO: 408).

In some embodiments, the nucleic acid sequence comprises at least one promoter. In some embodiments, the at least one promoter is a constitutive promoter or a tissue-specific promoter. In some embodiments, the at least one promoter is selected from the group consisting of an opsin promoter, an EFS promoter, and a combination thereof. In some embodiments, the nucleic acid sequence comprises two promoters. In one embodiment, the two promoters are an opsin promoter driving expression of the replacement rhodopsin protein and an EFS promoter driving expression of the PUF or PUMBY-based RNA-binding protein fused to a second RNA-binding protein which is an effector protein such as ZC3H12A.

In some embodiments disclosed herein is a vector comprising the knockdown replacement compositions disclosed herein. In some embodiments, the vector is selected from the group consisting of: adeno-associated virus, retrovirus, lentivirus, adenovirus, nanoparticle, micelle, liposome, lipoplex, polymersome, polyplex, and dendrimer. In some embodiments disclosed herein is a cell comprising the vectors disclosed herein.

In some embodiments of the compositions disclosed herein, the RNA-binding polypeptide is a first RNA-binding polypeptide, and the nucleic acid sequence encodes a second RNA-binding polypeptide which binds RNA in a manner in which it associates with RNA. In some embodiments, the second RNA-binding polypeptide associates with RNA in a manner in which it cleaves RNA. In some embodiments, the second RNA-binding polypeptide is selected from the group consisting of: RNAse1, RNAse4, RNAse6, RNAse7, RNAse8, RNAse2, RNAse6PL, RNAseL, RNAseT2, RNAse11, RNAseT2-like, NOB1, ENDOV, ENDOG, ENDOD1, hFEN1, hSLFN14, hLACTB2, APEX2, ANG, HRSP12, ZC3H12A, RIDA, PDL6, NTHL, KIAA0391, APEX1, AGO2, EXOG, ZC3H12D, ERN2, PELO, YBEY, CPSF4L, hCG_2002731, ERCC1, RAC1, RAA1, RAB1, DNA2, FLJ35220, FLJ13173, ERCC4, Rnase1(K41R), Rnase1(K41R, D121E), Rnase1(K41R, D121E, H119N), Rnase1(H119N), Rnase1(R39D, N67D, N88A, G89D, R91D, H119N), Rnase1(R39D, N67D, N88A, G89D, R91D, H119N, K41R, D121E), Rnase1(R39D, N67D, N88A, G89D, R91D), TENM1, TENM2, RNAseK, TALEN, ZNF638, and hSMG6. In one embodiment, the second RNA-binding polypeptide is ZC3H12A.

In some embodiments of the compositions of the disclosure, the sequence comprising the gRNA further comprises a sequence encoding a promoter capable of expressing the gRNA in a eukaryotic cell.

In some embodiments of the compositions of the disclosure, the gRNA comprises a spacer sequence comprising ACATGTAGATGACAAAAGACTCGTTG (SEQ ID NO: 465), TGAAGATGTAGAATGCCACGCTGGCG (SEQ ID NO: 409), or ACTGCTTGTTCATCATGATATAGATG (SEQ ID NO: 466).

In some embodiments of the compositions of the disclosure, the eukaryotic cell is an animal cell. In some embodiments, the animal cell is a mammalian cell. In some embodiments, the animal cell is a human cell.

In some embodiments of the compositions of the disclosure, the promoter is a constitutively active promoter. In some embodiments, the promoter sequence is isolated or derived from a promoter capable of driving expression of an RNA polymerase. In some embodiments, the promoter sequence is a Pol II promoter. In some embodiments, the promoter sequence is isolated or derived from a U6 promoter. In some embodiments, the promoter is a sequence isolated or derived from a promoter capable of driving expression of a transfer RNA (tRNA). In some embodiments, the promoter is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter is isolated or derived from a valine tRNA promoter.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides, 22 nucleotides, 23 nucleotides, 24 nucleotides, 25 nucleotides, 26 nucleotides, 27 nucleotides, 28 nucleotides, or 29 nucleotides. In some embodiments, the spacer sequence comprises or consists of 26 nucleotides. In some embodiments, the spacer sequence is non-processed and comprises or consists of 30 nucleotides. In some embodiments the non-processed spacer sequence comprises or consists of 30-36 nucleotides.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence.

In some embodiments of the compositions of the disclosure, the gRNA does not bind or does not selectively bind to a second sequence within the RNA molecule.

In some embodiments of the compositions of the disclosure, an RNA genome or an RNA transcriptome comprises the RNA molecule.

In some embodiments of the compositions of the disclosure, the first RNA binding protein comprises a CRISPR-Cas protein. In some embodiments, the CRISPR-Cas protein is a Type II CRISPR-Cas protein. In some embodiments, the first RNA binding protein comprises a Cas9 polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments, the pathogenic RNA comprises the target RNA, and/or the target RNA is associated with the pathogenic RNA. In some embodiments, the pathogenic RNA encodes gain-or-loss-of-function mutations.

In some embodiments of the compositions of the disclosure, the RNA binding protein comprises a CRISPR-Cas protein. In some embodiments, the CRISPR-Cas protein is a Type V CRISPR-Cas protein. In some embodiments, the RNA binding protein comprises a Cpf1 polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, the RNA binding protein comprises a CRISPR-Cas protein. In some embodiments, the CRISPR-Cas protein is a Type VI CRISPR-Cas protein. In some embodiments, the RNA binding protein comprises a Cas13 polypeptide or an RNA-binding portion thereof. In some embodiments, the RNA binding protein comprises a Cas13d polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, the RNA binding protein is a non-guided RNA binding protein. In some embodiments, the non-guided RNA binding protein comprises a Pumilio and FBF (PUF) protein or an RNA binding portion thereof. In some embodiments, the RNA binding protein comprises a Pumilio-based assembly (PUMBY) protein or an RNA binding portion thereof.

In some embodiments of the compositions of the disclosure, the RNA binding protein does not require multimerization for RNA-binding activity. In some embodiments, the RNA binding protein is not a monomer of a multimer complex. In some embodiments, a multimer protein complex does not comprise the RNA binding protein.

In some embodiments of the compositions of the disclosure, the RNA binding protein selectively binds to a target sequence within the RNA molecule. In some embodiments, the RNA binding protein does not comprise an affinity for a second sequence within the RNA molecule. In some embodiments, the RNA binding protein does not comprise a high affinity for or selectively bind a second sequence within the RNA molecule.

In some embodiments of the compositions of the disclosure, an RNA genome or an RNA transcriptome comprises the RNA molecule.

In some embodiments of the compositions of the disclosure, the RNA binding protein comprises between 2 and 1300 amino acids, inclusive of the endpoints.

In some embodiments of the compositions of the disclosure, the sequence encoding the RNA binding protein further comprises a sequence encoding a nuclear localization signal (NLS), a nuclear export signal (NES) or tag. In some embodiments, the sequence encoding a nuclear localization signal (NLS) is positioned at the N-terminus of the sequence encoding the RNA binding protein. In some embodiments, the RNA binding protein comprises an NLS at a C-terminus of the protein.

In some embodiments of the compositions of the disclosure, the sequence encoding the RNA binding protein further comprises a first sequence encoding a first NLS and a second sequence encoding a second NLS. In some embodiments, the sequence encoding the first NLS or the second NLS is positioned at the N-terminus of the sequence encoding the RNA binding protein. In some embodiments, the RNA binding protein comprises the first NLS or the second NLS at a C-terminus of the protein.

In some embodiments of the compositions of the disclosure, the composition further comprises a second RNA binding protein. In some embodiments, the second RNA binding protein comprises or consists of a nuclease domain. In some embodiments, the second RNA binding protein binds RNA in a manner in which it associates with RNA. In some embodiments, the second RNA binding protein associates with RNA in a manner in which it cleaves RNA. In some embodiments of the compositions of the disclosure, the sequence encoding the second RNA binding protein comprises or consists of an RNAse.

In some embodiments, the compositions of the disclosure are used in methods for treating a subject in need thereof, the methods comprising contacting a target RNA with a nucleic acid sequence encoding the knockdown RNA and replacement protein.

In some embodiments of the compositions disclosed herein are used in a method for reducing the level of expression of a pathogenic target RNA molecule or a protein encoded by the pathogenic RNA molecule and replacing gain-or-loss-of-function mutations caused by the pathogenic target RNA with a therapeutic replacement protein, the method comprising contacting the compositions disclosed herein and the pathogenic target RNA molecule comprising a target RNA sequence under conditions suitable for binding of the RNA binding protein to the target RNA sequence, wherein the level of expression of the pathogenic target RNA is reduced, and wherein the expression of the pathogenic target RNA is replaced with expression of a therapeutic replacement protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E are schematic diagrams of exemplary embodiments of compositions of the disclosure that depict nucleic acid sequence designs that promote simultaneous knockdown and replacement of pathogenic RNAs. Nucleic acid sequences A-E each describe exemplary vector sequences. In these embodiments, a polymerase II (“Pol II”) promoter drives expression of the RNA-targeting protein and a polymerase III promoter (“Pol III”) drives expression of the optional single guide RNA (“sgRNA”) in vectors that also encode a CRISPR-associated (Cas) RNA-targeting protein. The replacement protein is provided either by a second polymerase II promoter or via the same promoter that drives the RNA-targeting protein. In the case of a single polymerase II promoter system, the replacement gene and the RNA knockdown system are separated by either a 2A site or an internal ribosome entry site (IRES).

FIG. 2 is a schematic diagram of embodiments of therapeutic compositions and methods of the disclosure involving the knockdown and replace vector. Certain schematic vector designs are packaged in a delivery vehicle such as adeno-associated virus (AAV) and delivered to target tissue in a manner determined by AAV serotype and administration method. Once present in the target tissue, the therapeutic simultaneously replaces the mutated RNA and encoded protein while destroying the mutated RNA.

FIG. 3 is a plasmid map showing an exemplary configuration of pmirGlo designed for a luciferase reporter assay for detecting knockdown effect of the compositions disclosed herein.

FIG. 4 is a plasmid map showing a PUMBY-based knockdown and replacement embodiment of the compositions disclosed herein.

FIG. 5 is a plasmid map showing a PUF-based knockdown and replacement embodiment of the compositions disclosed herein

FIG. 6A-6C show embodiments of the compositions disclosed herein. FIG. 6A shows a schematic diagram of exemplary embodiments of compositions of the disclosure that depict nucleic acid sequence designs encoding PUF or PUMBY-based RNA-binding-effector fusion proteins. FIGS. 6B-6C show knockdown of Rhodopsin target RNA and replacement of the target RNA with “hardened” rhodopsin.

FIGS. 7A-7B show knockdown of Rhodopsin target RNA and replacement of the target RNA with “hardened” rhodopsin.

FIG. 8 shows a luciferase assay PUF-targeting Rhodopsin knockdown screen compared to no targeting.

### DETAILED DESCRIPTION

The disclosure provides a therapeutic combination of RNA-targeting and gene replacement. In particular, the disclosure provides compositions and methods for specifically targeting and knocking down pathogenic RNA molecules which lead to toxic gain-or-loss-of-function mutations in a sequence-specific manner while also replacing the targeted, and knocked down, gene with the corresponding therapeutic gene. In one embodiment, the pathogenic RNA comprises a target RNA sequence. In one embodiment, the pathogenic RNA comprises a target RNA sequence but the target RNA sequence does not comprise the gain-or-loss-of-function mutations. In another embodiment, the target RNA is in non-coding RNA. In a further embodiment, the pathogenic RNA comprises one or more additional target RNAs. In particular, the disclosure provides a composition comprising a nucleic acid sequence encoding a target RNA knockdown and replacement therapeutic comprising (a) an RNA-binding polypeptide or portion thereof, and (b) a therapeutic protein, wherein the RNA-binding polypeptide binds and cleaves a target RNA, wherein a pathogenic RNA comprises the target RNA, and wherein the therapeutic protein is a wild-type replacement of the pathogenic RNA or protein encoded by the pathogenic RNA. The disclosure provides vectors, compositions and cells comprising the knockdown and replacement compositions. The disclosure provides methods of using the knockdown and replacement systems, the RNA-guided (such as CRISPR/Cas-based) or non-RNA-guided (PUF or PUMBY-based) RNA-binding proteins fusions, guide RNAs (gRNAs) corresponding to RNA-guided CRISPR/Cas proteins, therapeutic replacement genes or portions thereof, vectors, compositions and cells of the disclosure to treat a disease or disorder. The compositions also provide particular target RNA sequences or particular targeting RNA sequences (e.g., a particular gRNA spacer sequence).

The compositions and methods of the disclosure provide a combined knockdown and therapeutic effect. Accordingly, the compositions comprise a nucleic acid sequence encoding 1) an RNA-binding polypeptide (RBP) or RNA-binding domain (RBD), capable of cleavage of a pathogenic RNA comprising a target RNA sequence, and 2) a replacement therapeutic protein. In some embodiments, the replacement therapeutic protein is the wild-type protein of the pathogenic target RNA or protein. In some embodiments, the therapeutic (e.g., wild-type) replacement protein replaces gain-or-loss-of-function mutations encoded by the pathogenic target RNA.

In some embodiments, the RNA-binding polypeptide is an RNA-guided RNA-binding polypeptide. In some embodiments, the RNA-guided RNA-binding polypeptide is a CRISPR/Cas protein and the nucleic acid sequence further comprises an gRNA sequence which corresponds to the target RNA and the CRISPR/Cas protein. In some embodiments, the RNA-binding polypeptide is not an RNA-guided RNA-binding polypeptide. In particular embodiments, the non-RNA-guided RNA-binding polypeptide is a PUF protein or a PUMBY protein or portion thereof. In some embodiments, the pathogenic RNA comprising the target RNA encodes gain-or-loss-of-function mutations.

In some embodiments, the pathogenic RNA encodes gain-or-loss-of-function mutations in the rhodopsin gene and the replacement gene encodes human rhodopsin. In some embodiments, the pathogenic rhodopsin RNA comprises a rhodopsin target RNA. In one embodiment, the rhodopsin target RNA sequence comprises GCCAGCGTGGCATTCTACATCTTC (SEQ ID NO: 406). In some embodiments, the rhodopsin target RNA comprises CAACGAGTCTTTTGTCATCTACATGT (SEQ ID NO: 462), CGCCAGCGTGGCATTCTACATCTTCA (SEQ ID NO: 463), or CATCTATATCATGATGAACAAGCAGT (SEQ ID NO: 464).

In another embodiment, the rhodopsin target RNA encodes an amino acid comprising ASVAFYIF (SEQ ID NO: 407). In one embodiment, the rhodopsin target RNA encodes an amino acid comprising ASVAFYIF (SEQ ID NO: 407) at e.g., position 269 to 276. In another embodiment, the target RNA encodes an amino acid comprising YASVAFYIFT (SEQ ID NO: 486). In another embodiment, the target RNA encodes an amino acid comprising YASVAFYIFT (SEQ ID NO: 486) at e.g., positions 268 to 277.

In some embodiments, the replacement gene encodes “hardened” rhodopsin. “Hardened” rhodopsin is an engineered wild-type rhodopsin the expression of which is engineered to be incapable of knockdown using the compositions disclosed herein. In one embodiment, a “hardened” rhodopsin nucleic acid sequence comprising at least one mismatch. In another embodiment, a “hardened” rhodopsin nucleic acid sequence comprising two or more mismatches. In one embodiment, the “hardened” rhodopsin is encoded by a nucleic acid sequence which does not comprise the rhodopsin target RNA comprising GCCAGCGTGGCATTCTACATCTTC SEQ ID NO: 406. In another embodiment, the “hardened” rhodopsin is encoded by a nucleic acid sequence comprising GCTTCCGTAGCTTTTTATATTTTT (SEQ ID NO: 408). In some embodiments, the spacer sequence of the gRNA is a sequence which is complementary to the rhodopsin target RNA. In one embodiment, the spacer sequence targeting the rhodopsin target RNA is ACATGTAGATGACAAAAGACTCGTTG (SEQ ID NO: 465), TGAAGATGTAGAATGCCACGCTGGCG (SEQ ID NO: 409), or ACTGCTTGTTCATCATGATATAGATG (SEQ ID NO 466).

### Guide RNAs

The terms guide RNA (gRNA) and single guide RNA (sgRNA) are used interchangeably throughout the disclosure.

Guide RNAs (gRNAs) of the disclosure may comprise of a spacer sequence and a scaffolding and/or a “direct repeat” (DR) sequence. In some embodiments, a guide RNA is a single guide RNA (sgRNA) comprising a contiguous spacer sequence and scaffolding sequence. In some embodiments, the spacer sequence and the scaffolding sequence are not contiguous. In some embodiments, a scaffold sequence comprises a “direct repeat” (DR) sequence. In some embodiments, the gRNA comprises a DR sequence. DR sequences refer to the repetitive sequences in the CRISPR locus (naturally-occurring in a bacterial genome or plasmid) that are interspersed with the spacer sequences. It is well known that one would be able to infer the DR sequence of a corresponding Cas protein if the sequence of the associated CRISPR locus is known. In some embodiments, a guide RNA comprises a direct repeat (DR) sequence and a spacer sequence. In some embodiments, a sequence encoding a guide RNA or single guide RNA of the disclosure comprises or consists of a spacer sequence and a scaffolding sequence and/or a DR sequence, that are separated by a linker sequence. In some embodiments, the linker sequence may comprise or consist of 1, 2, 3, 4, 5, 6, 7, 8, 9, 10, 15, 20, 25, 30, 35, 40, 45, 50 or any number of nucleotides in between. In some embodiments, the linker sequence may comprise at least 1, 2, 3, 4, 5, 6, 7, 8, 9, 10, 15, 20, 25, 30, 35, 40, 45, 50 or any number of nucleotides in between. In some embodiments, the scaffold sequence is a Cas9 scaffold sequence. In some embodiments, the DR sequence is a Cas13d sequence.

In one embodiment, the gRNA that hybridizes with the one or more target RNA molecules in a Cas 13d-mediated manner includes one or more direct repeat (DR) sequences, one or more spacer sequences, such as, e.g., one or more sequences comprising an array of DR-spacer-DR-spacer. In one embodiment, a plurality of gRNAs are generated from a single array, wherein each gRNA can be different, for example target different RNAs or target multiple regions of a single RNA, or combinations thereof. In some embodiments, an isolated gRNA includes one or more direct repeat (DR) sequences, such as an unprocessed (e.g., about 36 nt) or processed DR (e.g., about 30 nt). In some embodiments, a gRNA can further include one or more spacer sequences specific for (e.g., is complementary to) the target RNA. In certain such embodiments, multiple pol III promoters can be used to drive multiple gRNAs, spacers and/or DRs. In one embodiment, a guide array comprises a DR (about 36nt)-spacer (about 30nt)-DR (about 36nt)-spacer (about 30nt)-DR (about 36nt).

Guide RNAs (gRNAs) of the disclosure may comprise non-naturally occurring nucleotides. In some embodiments, a guide RNA of the disclosure or a sequence encoding the guide RNA comprises or consists of modified or synthetic RNA nucleotides. Exemplary modified RNA nucleotides include, but are not limited to, pseudouridine (Ψ), dihydrouridine (D), inosine (I), and 7-methylguanosine (m7G), hypoxanthine, xanthine, xanthosine, 7-methylguanine, 5, 6-Dihydrouracil, 5-methylcytosine, 5-methylcytidine, 5-hydropxymethylcytosine, isoguanine, and isocytosine.

Guide RNAs (gRNAs) of the disclosure may bind modified RNA within a target sequence. Within a target sequence, guide RNAs (gRNAs) of the disclosure may bind modified or mutated (e.g., pathogenic) RNA. Exemplary epigenetically or post-transcriptionally modified RNA include, but are not limited to, 2′-O-Methylation (2′-OMe) (2′-O-methylation occurs on the oxygen of the free 2′-OH of the ribose moiety), N6-methyladenosine (m6A), and 5-methylcytosine (m5C).

In some embodiments of the compositions of the disclosure, a guide RNA of the disclosure comprises at least one sequence encoding a non-coding C/D box small nucleolar RNA (snoRNA) sequence. In some embodiments, the snoRNA sequence comprises at least one sequence that is complementary to the target RNA, wherein the target sequence of the RNA molecule comprises at least one 2′-OMe. In some embodiments, the snoRNA sequence comprises at least one sequence that is complementary to the target RNA, wherein the at least one sequence that is complementary to the target RNA comprises a box C motif (RUGAUGA) and a box D motif (CUGA).

Spacer sequences of the disclosure bind to the target sequence of an RNA molecule. In some embodiments, spacer sequences of the disclosure bind to pathogenic target RNA.

Spacer sequences of the disclosure may comprise a CRISPR RNA (crRNA). Spacer sequences of the disclosure comprise or consist of a sequence having sufficient complementarity to a target sequence of an RNA molecule to bind selectively to the target sequence. Upon binding to a target sequence of an RNA molecule, the spacer sequence may guide one or more of a scaffolding sequence and a fusion protein to the RNA molecule. In some embodiments, a sequence having sufficient complementarity to a target sequence of an RNA molecule to bind selectively to the target sequence has at least 50%, 55%, 60%, 65%, 70%, 75%, 80%, 85%, 90%, 95%, 96, 97%, 98%, 99%, or any percentage identity in between to the target sequence. In some embodiments, a sequence having sufficient complementarity to a target sequence of an RNA molecule to bind selectively to the target sequence has 100% identity the target sequence.

Scaffolding sequences of the disclosure bind the first RNA-binding polypeptide of the disclosure. Scaffolding sequences of the disclosure may comprise a trans acting RNA (tracrRNA). Scaffolding sequences of the disclosure comprise or consist of a sequence having sufficient complementarity to a target sequence of an RNA molecule to bind selectively to the target sequence. Upon binding to a target sequence of an RNA molecule, the scaffolding sequence may guide a fusion protein to the RNA molecule. In some embodiments, a sequence having sufficient complementarity to a target sequence of an RNA molecule to bind selectively to the target sequence has at least 50%, 55%, 60%, 65%, 70%, 75%, 80%, 85%, 90%, 95%, 96, 97%, 98%, 99%, or any percentage identity in between to the target sequence. In some embodiments, a sequence having sufficient complementarity to a target sequence of an RNA molecule to bind selectively to the target sequence has 100% identity the target sequence. Alternatively, or in addition, in some embodiments, scaffolding sequences of the disclosure comprise or consist of a sequence that binds to a first RNA binding protein or a second RNA binding protein of a fusion protein of the disclosure. In some embodiments, scaffolding sequences of the disclosure comprise a secondary structure or a tertiary structure. Exemplary secondary structures include, but are not limited to, a helix, a stem loop, a bulge, a tetraloop and a pseudoknot. Exemplary tertiary structures include, but are not limited to, an A-form of a helix, a B-form of a helix, and a Z-form of a helix. Exemplary tertiary structures include, but are not limited to, a twisted or helicized stem loop. Exemplary tertiary structures include, but are not limited to, a twisted or helicized pseudoknot. In some embodiments, scaffolding sequences of the disclosure comprise at least one secondary structure or at least one tertiary structure. In some embodiments, scaffolding sequences of the disclosure comprise one or more secondary structure(s) or one or more tertiary structure(s).

In some embodiments of the compositions of the disclosure, a guide RNA or a portion thereof selectively binds to a tetraloop motif in an RNA molecule of the disclosure. In some embodiments, a target sequence of an RNA molecule comprises a tetraloop motif. In some embodiments, the tetraloop motif is a “GRNA” motif comprising or consisting of one or more of the sequences of GAAA, GUGA, GCAA or GAGA.

In some embodiments of the compositions of the disclosure, a guide RNA or a portion thereof that binds to a target sequence of an RNA molecule hybridizes to the target sequence of the RNA molecule. In some embodiments, a guide RNA or a portion thereof that binds to a first RNA binding protein or to a second RNA binding protein covalently binds to the first RNA binding protein or to the second RNA binding protein. In some embodiments, a guide RNA or a portion thereof that binds to a first RNA binding protein or to a second RNA binding protein non-covalently binds to the first RNA binding protein or to the second RNA binding protein.

In some embodiments of the compositions of the disclosure, a guide RNA or a portion thereof comprises or consists of between 10 and 100 nucleotides, inclusive of the endpoints. In some embodiments, a spacer sequence of the disclosure comprises or consists of between 10 and 30 nucleotides, inclusive of the endpoints. In some embodiments, a spacer sequence of the disclosure comprises or consists of 15, 16, 17, 18, 19, 20, 21, 22, 23, 24, 25, 26, 27, 28, 29 or 30 nucleotides. In some embodiments, the spacer sequence of the disclosure comprises or consists of 20 nucleotides. In some embodiments, the spacer sequence of the disclosure comprises or consists of 21 nucleotides. In some embodiments, the spacer sequence of the disclosure comprises or consists of 26 nucleotides.

Guide molecules generally exist in various states of processing. In one example, an unprocessed guide RNA is 36nt of DR followed by 30-32 nt of spacer. The guide RNA is processed (truncated/modified) by Cas 13d itself or other RNases into the shorter “mature” form. In some embodiments, an unprocessed guide sequence is about, or at least about 30, 35, 40, 45, 50, 55, 60, 61, 62, 63, 64, 65, 66, 67, 68, 69, 70, 71, 72, 73, 74, 75, or more nucleotides (nt) in length. In some embodiments, a processed guide sequence is about 44 to 60 nt (such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nt). In some embodiments, an unprocessed spacer is about 28-32 nt long (such as 25, 26, 27, 28, 29, 30, 31, 32, 33, 34, or 35 nt) while the mature (processed) spacer can be about 10 to 30 nt, 10 to 25 nt, 14 to 25 nt, 20 to 22 nt, or 14-30 nt (such as 10, 11, 12, 13, 14, 15, 16, 17, 18, 19, 20, 21, 22, 23, 24, 25, 26, 27, 28, 29, 30, 31, 32, 33, 34, or 35 nt). In some embodiments, an unprocessed DR is about 36 nt (such as 30, 31, 32, 33, 34, 35, 36, 37, 38, 39, 40 or 41 nt), while the processed DR is about 30 nt (such as 25, 26, 27, 28, 29, 30, 31, 32, 33, 34, or 35 nt). In some embodiments, a DR sequence is truncated by 1-10 nucleotides (such as 1, 2, 3, 4, 5, 6, 7, 8, 9, or 10 nucleotides at e.g., the 5′ end in order to be expressed as mature pre-processed guide RNAs.

In some embodiments, a scaffold sequence, such as e.g., a Cas9 scaffold sequence, of the disclosure comprises or consists of between 10 and 100 nucleotides, inclusive of the endpoints. In some embodiments, a scaffold sequence of the disclosure comprises or consists of 30, 35, 40, 45, 50, 55, 60, 65, 70, 76, 80, 87, 90, 95, 100 or any number of nucleotides in between. In some embodiments, the scaffold sequence of the disclosure comprises or consists of between 85 and 95 nucleotides, inclusive of the endpoints. In some embodiments, the scaffold sequence of the disclosure comprises or consists of 85 nucleotides. In some embodiments, the scaffold sequence of the disclosure comprises or consists of 90 nucleotides. In some embodiments, the scaffold sequence of the disclosure comprises or consists of 93 nucleotides. In some embodiments of the compositions of the disclosure, the sequence comprising the gRNA further comprises a scaffold sequence that specifically binds to the first RNA binding protein. In some embodiments, the scaffold sequence comprises a stem-loop structure. In some embodiments, the scaffold sequence comprises or consists of 90 nucleotides. In some embodiments, the scaffold sequence comprises or consists of 93 nucleotides. In some embodiments, the scaffold sequence comprises or consists of the sequence GUUUAAGAGCUAUGCUGGAAACAGCAUAGCAAGUUUAAAUAAGGCUAGUCCGUU AUCAACUUGAAAAAGUGGCACCGAGUCGGUGCUUUUUUU (SEQ ID NO: 403). In some embodiments, the scaffold sequence comprises or consists of the sequence GGACAGCAUAGCAAGUUAAAAUAAGGCUAGUCCGUUAUCAACUUGAAAAAGUGG CACCGAGUCGGUGCUUUUU (SEQ ID NO: 404). In some embodiments, the scaffold sequence comprises or consists of the sequence GUUUUAGAGCUAGAAAUAGCAAGUUAAAAUAAGGCUAGUCCGUUAUCAACUUGA AAAAGUGGCACCGAGUCGGUGCUUUUUUU (SEQ ID NO: 405).

In some embodiments of the compositions of the disclosure, a guide RNA or a portion thereof does not comprise a nuclear localization sequence (NLS).

In some embodiments of the compositions of the disclosure, a guide RNA or a portion thereof does not comprise a sequence complementary to a protospacer adjacent motif (PAM).

Therapeutic or pharmaceutical compositions of the disclosure do not comprise a PAMmer oligonucleotide. In other embodiments, optionally, non-therapeutic or non-pharmaceutical compositions may comprise a PAMmer oligonucleotide. The term “PAMmer” refers to an oligonucleotide comprising a PAM sequence that is capable of interacting with a guide nucleotide sequence-programmable RNA binding protein. Non-limiting examples of PAMmers are described in O'Connell et al. Nature 516, pages 263-266 (2014), incorporated herein by reference. A PAM sequence refers to a protospacer adjacent motif comprising about 2 to about 10 nucleotides. PAM sequences are specific to the guide nucleotide sequence-programmable RNA binding protein with which they interact and are known in the art. For example, Streptococcus pyogenes PAM has the sequence 5′-NGG-3′, where “N” is any nucleobase followed by two guanine (“G”) nucleobases. Cas9 of Francisella novicida recognizes the canonical PAM sequence 5′-NGG-3′, but has been engineered to recognize the PAM 5′-YG-3′ (where “Y” is a pyrimidine), thus adding to the range of possible Cas9 targets. The Cpf1 nuclease of Francisella novicida recognizes the PAM 5′-TTTN-3′ or 5′-YTN-3′.

In some embodiments of the compositions of the disclosure, a guide RNA or a portion thereof comprises a sequence complementary to a protospacer flanking sequence (PFS). In some embodiments, including those wherein a guide RNA or a portion thereof comprises a sequence complementary to a PFS, the first RNA binding protein may comprise a sequence isolated or derived from a Cas13 protein. In some embodiments, including those wherein a guide RNA or a portion thereof comprises a sequence complementary to a PFS, the first RNA binding protein may comprise a sequence encoding a Cas13 protein or an RNA-binding portion thereof. In some embodiments, the guide RNA or a portion thereof does not comprise a sequence complementary to a PFS.

In some embodiments of the compositions of the disclosure, guide RNA sequence of the disclosure comprises a promoter sequence to drive expression of the guide RNA. In some embodiments, a vector comprising a guide RNA sequence of the disclosure comprises a promoter sequence to drive expression of the guide RNA. In some embodiments, the promoter to drive expression of the guide RNA is a constitutive promoter. In some embodiments, the promoter sequence is an inducible promoter. In some embodiments, the promoter is a sequence is a tissue-specific and/or cell-type specific promoter. In some embodiments, the promoter is a hybrid or a recombinant promoter. In some embodiments, the promoter is a promoter capable of expressing the guide RNA in a mammalian cell. In some embodiments, the promoter is a promoter capable of expressing the guide RNA in a human cell. In some embodiments, the promoter is a promoter capable of expressing the guide RNA and restricting the guide RNA to the nucleus of the cell. In some embodiments, the promoter is a human RNA polymerase promoter or a sequence isolated or derived from a sequence encoding a human RNA polymerase promoter. In some embodiments, the promoter is a U6 promoter or a sequence isolated or derived from a sequence encoding a U6 promoter. In some embodiments, the promoter is a human tRNA promoter or a sequence isolated or derived from a sequence encoding a human tRNA promoter. In some embodiments, the promoter is a human valine tRNA promoter or a sequence isolated or derived from a sequence encoding a human valine tRNA promoter.

In some embodiments of the compositions of the disclosure, a promoter to drive expression of the guide RNA further comprises a regulatory element. In some embodiments, a vector comprising a promoter sequence to drive expression of the guide RNA further comprises a regulatory element. In some embodiments, a regulatory element enhances expression of the guide RNA. Exemplary regulatory elements include, but are not limited to, an enhancer element, an intron, an exon, or a combination thereof.

In some embodiments of the compositions of the disclosure, a vector of the disclosure comprises one or more of a sequence encoding a guide RNA, a promoter sequence to drive expression of the guide RNA and a sequence encoding a regulatory element. In some embodiments of the compositions of the disclosure, the vector further comprises a sequence encoding a fusion protein of the disclosure.

In some embodiments of the compositions of the disclosure, gRNAs correspond to target RNA molecules and an RNA-guided RNA binding protein. In some embodiments, the gRNAs correspond to an RNA-guided RNA binding fusion protein, wherein the fusion protein comprises first and second RNA binding proteins. In some embodiments, along a sequence encoding the RNA-binding fusion protein, the sequence encoding the first RNA binding protein is positioned 5′ of the sequence encoding the second RNA binding protein. In some embodiments, along a sequence encoding the fusion protein, the sequence encoding the first RNA binding protein is positioned 3′ of the sequence encoding the second RNA binding protein.

In some embodiments of the compositions of the disclosure, the sequence encoding the first RNA binding protein comprises a sequence isolated or derived from a protein capable of binding an RNA molecule. In some embodiments, the sequence encoding the first RNA binding protein comprises a sequence isolated or derived from a protein capable of selectively binding an RNA molecule and not binding a DNA molecule, a mammalian DNA molecule or any DNA molecule. In some embodiments, the sequence encoding the first RNA binding protein comprises a sequence isolated or derived from a protein capable of binding an RNA molecule and inducing a break in the RNA molecule. In some embodiments, the sequence encoding the first RNA binding protein comprises a sequence isolated or derived from a protein capable of binding an RNA molecule, inducing a break in the RNA molecule, and not binding a DNA molecule, a mammalian DNA molecule or any DNA molecule. In some embodiments, the sequence encoding the first RNA binding protein comprises a sequence isolated or derived from a protein capable of binding an RNA molecule, inducing a break in the RNA molecule, and neither binding nor inducing a break in a DNA molecule, a mammalian DNA molecule or any DNA molecule.

In some embodiments of the compositions of the disclosure, the sequence encoding the first RNA binding protein comprises a sequence isolated or derived from a protein with no DNA nuclease activity.

In some embodiments of the compositions of the disclosure, the sequence encoding the first RNA binding protein comprises a sequence isolated or derived from a protein having DNA nuclease activity, wherein the DNA nuclease activity does not induce a break in a DNA molecule, a mammalian DNA molecule or any DNA molecule when a composition of the disclosure is contacted to an RNA molecule or introduced into a cell or into a subject of the disclosure.

In some embodiments of the compositions of the disclosure, the sequence encoding the first RNA binding protein comprises a sequence isolated or derived from a protein having DNA nuclease activity, wherein the DNA nuclease activity is inactivated and wherein the DNA nuclease activity does not induce a break in a DNA molecule, a mammalian DNA molecule or any DNA molecule when a composition of the disclosure is contacted to an RNA molecule or introduced into a cell or into a subject of the disclosure. In some embodiments, the sequence encoding the first RNA binding protein comprises a mutation that inactivates or decreases the DNA nuclease activity to a level at which the DNA nuclease activity does not induce a break in a DNA molecule, a mammalian DNA molecule or any DNA molecule when a composition of the disclosure is contacted to an RNA molecule or introduced into a cell or into a subject of the disclosure. In some embodiments, the sequence encoding the first RNA binding protein comprises a mutation that inactivates or decreases the DNA nuclease activity and the mutation comprises one or more of a substitution, inversion, transposition, insertion, deletion, or any combination thereof to a nucleic acid sequence or amino acid sequence encoding the first RNA binding protein or a nuclease domain thereof.

In some embodiments of the compositions of the disclosure, the sequence encoding the RNA-guided RNA binding protein disclosed herein comprises a sequence isolated or derived from a CRISPR Cas protein. In some embodiments, the CRISPR Cas protein comprises a Type II CRISPR Cas protein. In some embodiments, the Type II CRISPR Cas protein comprises a Cas9 protein. Exemplary Cas9 proteins of the disclosure may be isolated or derived from any species, including, but not limited to, a bacteria or an archaea. Exemplary Cas9 proteins of the disclosure may be isolated or derived from any species, including, but not limited to, Streptococcus pyogenes, Haloferax mediteranii, Mycobacterium tuberculosis, Francisella tularensis subsp. novicida, Pasteurella multocida, Neisseria meningitidis, Campylobacter jejune, Streptococcus thermophilus, Campylobacter lari CF89-12, Mycoplasma gallisepticum str. F, Nitratifractor salsuginis str. DSM 16511, Parvibaculum lavamentivorans, Roseburia intestinalis, Neisseria cinerea, a Gluconacetobacter diazotrophicus, an Azospirillum B510, a Sphaerochaeta globus str. Buddy, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Treponema denticola, Legionella pneumophila str. Paris, Sutterella wadsworthensis, Corynebacter diphtherias, Streptococcus aureus, and Francisella novicida.

Exemplary wild type S. pyogenes Cas9 proteins of the disclosure may comprise or consist of the amino acid sequence of

### SEQ ID NO: 416.

Nuclease inactivated S. pyogenes Cas9 proteins may comprise a substitution of an Alanine (A) for an Aspartic Acid (D) at position 10 and an alanine (A) for a Histidine (H) at position 840. Exemplary nuclease inactivated S. pyogenes Cas9 proteins of the disclosure may comprise or consist of the amino acid sequence (D10A and H840A bolded and underlined) of SEQ ID NO: 417.

Nuclease inactivated S. pyogenes Cas9 proteins may comprise deletion of a RuvC nuclease domain or a portion thereof, an HNH domain, a DNAse active site, a ββα-metal fold or a portion thereof comprising a DNAse active site or any combination thereof.

Other exemplary Cas9 proteins or portions thereof may comprise or consist of the following amino acid sequences.

In some embodiments the Cas9 protein can be S. pyogenes Cas9 and may comprise or consist of the amino acid sequence of SEQ ID NO: 418.

In some embodiments the Cas9 protein can be S. aureus Cas9 and may comprise or consist of the amino acid sequence of SEQ ID NO: 419.

In some embodiments the Cas9 protein can be S. thermophiles CRISPR1 Cas9 and may comprise or consist of the amino acid sequence of SEQ ID NO: 420.

In some embodiments the Cas9 protein can be N. meningitidis Cas9 and may comprise or consist of the amino acid sequence of SEQ ID NO: 421.

In some embodiments the Cas9 protein can be Parvibaculum. lavamentivorans Cas9 and may comprise or consist of the amino acid sequence of SEQ ID NO: 422.

In some embodiments the Cas9 protein can be Corynebacter diphtheria Cas9 and may comprise or consist of the amino acid sequence of SEQ ID NO: 423.

In some embodiments the Cas9 protein can be Streptococcus pasteurianus Cas9 and may comprise or consist of the amino acid sequence of SEQ ID NO: 424.

In some embodiments the Cas9 protein can be Neisseria cinerea Cas9 and may comprise or consist of the amino acid sequence of SEQ ID NO: 425.

In some embodiments the Cas9 protein can be Campylobacter lari Cas9 and may comprise or consist of the amino acid sequence of SEQ ID NO: 426.

In some embodiments the Cas9 protein can be T. denticola Cas9 and may comprise or consist of the amino acid sequence of SEQ ID NO: 427.

In some embodiments the Cas9 protein can be S. mutans Cas9 and may comprise or consist of the amino acid sequence of SEQ ID NO: 428.

In some embodiments the Cas9 protein can be S. thermophilus CRISPR 3 Cas9 and may comprise or consist of the amino acid sequence of SEQ ID NO: 429.

In some embodiments the Cas9 protein can be C. jejuni Cas9 and may comprise or consist of the amino acid sequence of SEQ ID NO: 430.

In some embodiments the Cas9 protein can be P. multocida Cas9 and may comprise or consist of the amino acid sequence of SEQ ID NO: 431.

In some embodiments the Cas9 protein can be F. novicida Cas9 and may comprise or consist of the amino acid sequence of SEQ ID NO: 432.

In some embodiments the Cas9 protein can be Lactobacillus buchneri Cas9 and may comprise or consist of the amino acid sequence of SEQ ID NO: 433.

In some embodiments the Cas9 protein can be Listeria innocua Cas9 and may comprise or consist of the amino acid sequence of SEQ ID NO: 434.

In some embodiments the Cas9 protein can be L. pneumophilia Cas9 and may comprise or consist of the amino acid sequence of SEQ ID NO: 435.

In some embodiments the Cas9 protein can be N. lactamica Cas9 and may comprise or consist of the amino acid sequence of SEQ ID NO: 436.

In some embodiments the Cas9 protein can be N. meningitides Cas9 and may comprise or consist of the amino acid sequence of SEQ ID NO: 437.

In some embodiments the Cas9 protein can be B. longum Cas9 and may comprise or consist of the amino acid sequence of SEQ ID NO: 438.

In some embodiments the Cas9 protein can be A. muciniphila Cas9 and may comprise or consist of the amino acid sequence of SEQ ID NO: 439.

In some embodiments the Cas9 protein can be O. laneus Cas9 and may comprise or consist of the amino acid sequence of SEQ ID NO: 440.

In some embodiments of the compositions of the disclosure, the sequence encoding the first RNA binding protein comprises a sequence isolated or derived from a CRISPR Cas protein or portion thereof. In some embodiments, the CRISPR Cas protein comprises a Type V CRISPR Cas protein. In some embodiments, the Type V CRISPR Cas protein comprises a Cpf1 protein. Exemplary Cpf1 proteins of the disclosure may be isolated or derived from any species, including, but not limited to, a bacteria or an archaea. Exemplary Cpf1 proteins of the disclosure may be isolated or derived from any species, including, but not limited to, Francisella tularensis subsp. novicida, Acidaminococcus sp. BV3L6 and Lachnospiraceae bacterium sp. ND2006. Exemplary Cpf1 proteins of the disclosure may be nuclease inactivated.

Exemplary wild type Francisella tularensis subsp. Novicida Cpf1 (FnCpf1) proteins of the disclosure may comprise or consist of the amino acid sequence of SEQ ID NO: 441.

Exemplary wild type Lachnospiraceae bacterium sp. ND2006 Cpf1 (LbCpf1) proteins of the disclosure may comprise or consist of the amino acid sequence of SEQ ID NO: 442.

Exemplary wild type Acidaminococcus sp. BV3L6 Cpf1 (AsCpf1) proteins of the disclosure may comprise or consist of the amino acid sequence of SEQ ID NO: 443.

In some embodiments of the compositions of the disclosure, the sequence encoding the RNA binding protein comprises a sequence isolated or derived from a CRISPR Cas protein. In some embodiments, the CRISPR Cas protein comprises a Type VI CRISPR Cas protein or portion thereof. In some embodiments, the Type VI CRISPR Cas protein comprises a Cas13 protein or portion thereof. Exemplary Cas13 proteins of the disclosure may be isolated or derived from any species, including, but not limited to, a bacteria or an archaea. Exemplary Cas13 proteins of the disclosure may be isolated or derived from any species, including, but not limited to, Leptotrichia wadei, Listeria seeligeri serovar 1/2b (strain ATCC 35967 DSM 20751 CIP 100100 SLCC 3954), Lachnospiraceae bacterium, Clostridium aminophilum DSM 10710, Carnobacterium gallinarum DSM 4847, Paludibacter propionicigenes WB4, Listeria weihenstephanensis FSL R9-0317, Listeria weihenstephanensis FSL R9-0317, bacterium FSL M6-0635 (Listeria newyorkensis), Leptotrichia wadei F0279, Rhodobacter capsulatus SB 1003, Rhodobacter capsulatus R121, Rhodobacter capsulatus DE442 and Corynebacterium ulcerans. Exemplary Cas13 proteins of the disclosure may be DNA nuclease inactivated. Exemplary Cas13 proteins of the disclosure include, but are not limited to, Cas13a, Cas13b, Cas13c, Cas13d and orthologs thereof. Exemplary Cas13b proteins of the disclosure include, but are not limited to, subtypes 1 and 2 referred to herein as Csx27 and Csx28, respectively.

Exemplary Cas13a proteins include, but are not limited to:

| Cas13a | Cas13a | | | |
|---|---|---|---|---|
| number | abbreviation | Organism name | Accession number | Direct Repeat sequence |
| Cas13a1 | LshCas13a | Leptotrichia | WP_018451595.1 | CCACCCCAATATCGAAGGGGACTAA |
| | | shahii | | AAC (SEQ ID NO: 444) |
| Cas13a2 | LwaCas13a | Leptotrichia | WP_021746774.1 | GATTTAGACTACCCCAAAAACGAAG |
| | | wadei | | GGGACTAAAAC (SEQ ID NO: |
| | | | | 445) |
| Cas13a3 | LseCas13a | Listeria seeligeri | WP_012985477.1 | GTAAGAGACTACCTCTATATGAAAG |
| | | | | AGGACTAAAAC (SEQ ID NO: |
| | | | | 446) |
| Cas13a4 | LbmCas13a | Lachnospiraceae | WP_044921188.1 | GTATTGAGAAAAGCCAGATATAGTT |
| | | bacterium | | GGCAATAGAC (SEQ ID NO: 447) |
| | | MA2020 | | |
| Cas13a5 | LbnCas13a | Lachnospiraceae | WP_022785443.1 | GTTGATGAGAAGAGCCCAAGATAG |
| | | bacterium | | AGGGCAATAAC (SEQ ID NO: |
| | | NK4A179 | | 448) |
| Cas13a6 | CamCas13a | [Clostridium] | WP 031473346.1 | GTCTATTGCCCTCTATATCGGGCTGT |
| | | aminophilum | | TCTCCAAAC (SEQ ID NO: 449) |
| | | DSM 10710 | | |
| Cas13a7 | CgaCas13a | Camobacterium | WP_034560163.1 | ATTAAAGACTACCTCTAAATGTAAG |
| | | gallinarum DSM | | AGGACTATAAC (SEQ ID NO: |
| | | 4847 | | 450) |
| Cas13a8 | Cga2Cas13a | Camobacterium | WP_034563842.1 | AATATAAACTACCTCTAAATGTAAG |
| | | gallinarum DSM | | AGGACTATAAC (SEQ ID NO: |
| | | 4847 | | 451) |
| Cas13a9 | Pprcas13a | Paludibacter | WP_013443710.1 | CTTGTGGATTATCCCAAAATTGAAG |
| | | propionicigenes | | GGAACTACAAC (SEQ ID NO: |
| | | WB4 | | 452) |
| Cas13a10 | LweCas13a | Listeria | WP_036059185.1 | GATTTAGAGTACCTCAAAATAGAAG |
| | | weihenstephanen | | AGGTCTAAAAC (SEQ ID NO: |
| | | sis FSL R9-0317 | | 453) |
| Cas13a11 | LbfCas13a | Listeriaceae | WP_036091002.1 | GATTTAGAGTACCTCAAAACAAAAG |
| | | bacterium FSL | | AGGACTAAAAC (SEQ ID NO: |
| | | M6-0635 | | 454) |
| | | (Listeria | | |
| | | newyorkensis) | | |
| Cas13a12 | Lwa2cas13a | Leptotrichia | WP_021746774.1 | GATATAGATAACCCCAAAAACGAA |
| | | wadei F0279 | | GGGATCTAAAAC (SEQ ID NO: |
| | | | | 455) |
| Cas13a13 | RcsCas13a | Rhodobacter | WP_013067728.1 | GCCTCACATCACCGCCAAGACGACG |
| | | capsulatus SB | | GCGGACTGAAC (SEQ ID NO: 456) |
| | | 1003 | | |
| Cas13a14 | RcrCas13a | Rhodobacter | WP_023911507.1 | GCCTCACATCACCGCCAAGACGACG |
| | | capsulatus R121 | | GCGGACTGAAC (SEQ ID NO: |
| | | | | 457) |
| Cas13a15 | RcdCas13a | Rhodobacter | WP_023911507.1 | GCCTCACATCACCGCCAAGACGACG |
| | | capsulatus | | GCGGACTGAAC (SEQ ID NO: |
| | | DE442 | | 458) |

Exemplary wild type Cas13a proteins of the disclosure may comprise or consist of the amino acid sequence of SEQ ID NO: 459.

Exemplary Cas13b proteins include, but are not limited to:

| Species | Cas13b Accession | Cas13b Size (aa) |
|---|---|---|
| Paludibacter propionicigenes WB4 | WP_013446107.1 | 1155 |
| Prevotella sp. P5-60 | WP_044074780.1 | 1091 |
| Prevotella sp. P4-76 | WP_044072147.1 | 1091 |
| Prevotella sp. P5-125 | WP_044065294.1 | 1091 |
| Prevotella sp. P5-119 | WP_042518169.1 | 1091 |
| Capnocytophaga canimorsus Cc5 | WP_013997271.1 | 1200 |
| Phaeodactylibacter xiamenensis | WP_044218239.1 | 1132 |
| Porphyromonas gingivalis W83 | WP_005873511.1 | 1136 |
| Porphyromonas gingivalis F0570 | WP_021665475.1 | 1136 |
| Porphyromonas gingivalis ATCC 33277 | WP_012458151.1 | 1136 |
| Porphyromonas gingivalis F0185 | ERJ81987.1 | 1136 |
| Porphyromonas gingivalis F0185 | WP_021677657.1 | 1136 |
| Porphyromonas gingivalis SJD2 | WP_023846767.1 | 1136 |
| Porphyromonas gingivalis F0568 | ERJ65637.1 | 1136 |
| Porphyromonas gingivalis W4087 | ERJ87335.1 | 1136 |
| Porphyromonas gingivalis W4087 | WP_021680012.1 | 1136 |
| Porphyromonas gingivalis F0568 | WP_021663197.1 | 1136 |
| Porphyromonas gingivalis | WP_061156637.1 | 1136 |
| Porphyromonas gulae | WP_039445055.1 | 1136 |
| Bacteroides pyogenes F0041 | ERI81700.1 | 1116 |
| Bacteroides pyogenes JCM 10003 | WP_034542281.1 | 1116 |
| Alistipes sp. ZOR0009 | WP_047447901.1 | 954 |
| Flavobacterium branchiophilum FL-15 | WP_014084666.1 | 1151 |
| Prevotella sp. MA2016 | WP_036929175.1 | 1323 |
| Myroides odoratimimus CCUG 10230 | EHO06562.1 | 1160 |
| Myroides odoratimimus CCUG 3837 | EKB06014.1 | 1158 |
| Myroides odoratimimus CCUG 3837 | WP_006265509.1 | 1158 |
| Myroides odoratimimus CCUG 12901 | WP_006261414.1 | 1158 |
| Myroides odoratimimus CCUG 12901 | EHO08761.1 | 1158 |
| Myroides odoratimimus (NZ_CP013690.1) | WP_058700060.1 | 1160 |
| Bergeyella zoohelcum ATCC 43767 | EKB54193.1 | 1225 |
| Capnocytophaga cynodegmi | WP_041989581.1 | 1219 |
| Bergeyella zoohelcum ATCC 43767 | WP_002664492.1 | 1225 |
| Flavobacterium sp. 316 | WP_045968377.1 | 1156 |
| Psychroflexus torquis ATCC 700755 | WP_015024765.1 | 1146 |
| Flavobacterium columnare ATCC 49512 | WP_014165541.1 | 1180 |
| Flavobacterium columnare | WP_060381855.1 | 1214 |
| Flavobacterium columnare | WP_063744070.1 | 1214 |
| Flavobacterium columnare | WP_065213424.1 | 1215 |
| Chryseobacterium sp. YR477 | WP_047431796.1 | 1146 |
| Riemerella anatipestifer ATCC 11845 = DSM | WP_004919755.1 | 1096 |
| 15868 | | |
| Riemerella anatipestifer RA-CH-2 | WP_015345620.1 | 949 |
| Riemerella anatipestifer | WP_049354263.1 | 949 |
| Riemerella anatipestifer | WP_061710138.1 | 951 |
| Riemerella anatipestifer | WP_064970887.1 | 1096 |
| Prevotella saccharolytica F0055 | EKY00089.1 | 1151 |
| Prevotella saccharolytica JCM 17484 | WP_051522484.1 | 1152 |
| Prevotella buccae ATCC 33574 | EFU31981.1 | 1128 |
| Prevotella buccae ATCC 33574 | WP_004343973.1 | 1128 |
| Prevotella buccae D17 | WP004343581.1 | 1128 |
| Prevotella sp. MSX73 | WP_007412163.1 | 1128 |
| Prevotella pallens ATCC 700821 | EGQ18444.1 | 1126 |
| Prevotella pallens ATCC 700821 | WP_006044833.1 | 1126 |
| Prevotella intermedia ATCC 25611 = DSM 20706 | WP_036860899.1 | 1127 |
| Prevotella intermedia | WP_061868553.1 | 1121 |
| Prevotella intermedia 17 | AFJ07523.1 | 1135 |
| Prevotella intermedia | WP_050955369.1 | 1133 |
| Prevotella intermedia | BAU18623.1 | 1134 |
| Prevotella intermedia ZT | KJJ86756.1 | 1126 |
| Prevotella aurantiaca JCM 15754 | WP_025000926.1 | 1125 |
| Prevotella pleuritidis F0068 | WP_021584635.1 | 1140 |
| Prevotella pleuritidis JCM 14110 | WP_036931485.1 | 1117 |
| Prevotella falsenii DSM 22864 = JCM 15124 | WP_036884929.1 | 1134 |
| Porphyromonas gulae | WP_039418912.1 | 1176 |
| Porphyromonas sp. COT-052 OH4946 | WP_039428968.1 | 1176 |
| Porphyromonas gulae | WP_039442171.1 | 1175 |
| Porphyromonas gulae | WP_039431778.1 | 1176 |
| Porphyromonas gulae | WP_046201018.1 | 1176 |
| Porphyromonas gulae | WP_039434803.1 | 1176 |
| Porphyromonas gulae | WP_039419792.1 | 1120 |
| Porphyromonas gulae | WP_039426176.1 | 1120 |
| Porphyromonas gulae | WP_039437199.1 | 1120 |
| Porphyromonas gingivalis TDC60 | WP_013816155.1 | 1120 |
| Porphyromonas gingivalis ATCC 33277 | WP_012458414.1 | 1120 |
| Porphyromonas gingivalis A7A1-28 | WP_058019250.1 | 1176 |
| Porphyromonas gingivalis JCVI SC001 | EOA10535.1 | 1176 |
| Porphyromonas gingivalis W50 | WP_005874195.1 | 1176 |
| Porphyromonas gingivalis | WP_052912312.1 | 1176 |
| Porphyromonas gingivalis AJW4 | WP_053444417.1 | 1120 |
| Porphyromonas gingivalis | WP_039417390.1 | 1120 |
| Porphyromonas gingivalis | WP_061156470.1 | 1120 |

Exemplary wild type Bergeyella zoohelcum ATCC 43767 Cas13b (BzCas13b) proteins of the disclosure may comprise or consist of the amino acid sequence of SEQ ID NO: 460.

In some embodiments of the compositions of the disclosure, the sequence encoding the RNA binding protein comprises a sequence isolated or derived from a Cas13d protein. Cas13d is an effector of the type V-D CRISPR-Cas systems. In some embodiments, the Cas13d protein is an RNA-guided RNA endonuclease enzyme that can cut or bind RNA. In some embodiments, the Cas13d protein can include one or more higher eukaryotes and prokaryotes nucleotide-binding (HEPN) domains. In some embodiments, the Cas13d protein can include either a wild-type or mutated HEPN domain. In some embodiments, the Cas13d protein includes a mutated HEPN domain that cannot cut RNA but can process guide RNA. In some embodiments, the Cas13d protein does not require a protospacer flanking sequence. Also see WO Publication No. WO2019/040664 & US2019/0062724, which is incorporated herein by reference in its entirety, for further examples and sequences of Cas13d protein, without limitation.

In some embodiments, Cas13d sequences of the disclosure include without limitation SEQ ID NOS: 1-296 of WO 2019/040664, so numbered herein and included herewith.

SEQ ID NO: 1 is an exemplary Cas13d sequence from Eubacterium siraeum containing a HEP site.

SEQ ID NO: 2 is an exemplary Cas13d sequence from Eubacterium siraeum containing a mutated HEPN site.

SEQ ID NO: 3 is an exemplary Cas13d sequence from uncultured Ruminococcus sp. containing a HEPN site.

SEQ ID NO: 4 is an exemplary Cas13d sequence from uncultured Ruminococcus sp. containing a mutated HEPN site.

SEQ ID NO: 5 is an exemplary Cas13d sequence from Gut_metagenome_contig2791000549.

SEQ ID NO: 6 is an exemplary Cas13d sequence from Gut_metagenome_contig855000317

SEQ ID NO: 7 is an exemplary Cas13d sequence from Gut_metagenome_contig3389000027.

SEQ ID NO: 8 is an exemplary Cas13d sequence from Gut_metagenome_contig8061000170.

SEQ ID NO: 9 is an exemplary Cas13d sequence from Gut_metagenome_contigl509000299.

SEQ ID NO: 10 is an exemplary Cas13d sequence from Gut_metagenome_contig9549000591.

SEQ ID NO: 11 is an exemplary Cas13d sequence from Gut_metagenome_contig71000500.

SEQ ID NO: 12 is an exemplary Cas13d sequence from human gut metagenome.

SEQ ID NO: 13 is an exemplary Cas13d sequence from Gut_metagenome_contig3915000357.

SEQ ID NO: 14 is an exemplary Cas13d sequence from Gut_metagenome_contig4719000173.

SEQ ID NO: 15 is an exemplary Cas13d sequence from Gut_metagenome_contig6929000468.

SEQ ID NO: 16 is an exemplary Cas3d sequence from Gut_metagenome_contig7367000486.

SEQ ID NO: 17 is an exemplary Cas13d sequence from Gut_metagenome_contig7930000403.

SEQ ID NO: 18 is an exemplary Cas13d sequence from Gut_metagenome_contig993000527.

SEQ ID NO: 19 is an exemplary Cas13d sequence from Gut_metagenome_contig6552000639.

SEQ ID NO: 20 is an exemplary Cas13d sequence from Gut_metagenome_contig11932000246.

SEQ ID NO: 21 is an exemplary Cas13d sequence from Gut_metagenome_contigl2963000286.

SEQ ID NO: 22 is an exemplary Cas13d sequence from Gut_metagenome_contig2952000470.

SEQ ID NO: 23 is an exemplary Cas13d sequence from Gut_metagenome_contig451000394.

SEQ ID NO: 24 is an exemplary Cas13d sequence from Eubacterium_siraeum_DSM_15702.

SEQ ID NO: 25 is an exemplary Cas13d sequence from gut_metagenome_P19E0k2120140920,_c369000003.

SEQ ID NO: 26 is an exemplary Cas13d sequence from Gut_metagenome_contig7593000362.

SEQ ID NO: 27 is an exemplary Cas13d sequence from Gut_metagenome_contigl2619000055.

SEQ ID NO: 28 is an exemplary Cas13d sequence from Gut_metagenome_contigl405000151.

SEQ ID NO: 29 is an exemplary Cas13d sequence from Chicken_gut_metagenome_c298474.

SEQ ID NO: 30 is an exemplary Cas13d sequence from Gut_metagenome_contigl516000227.

SEQ ID NO: 31 is an exemplary Cas13d sequence from Gut_metagenome_contigl838000319.

SEQ ID NO: 32 is an exemplary Cas13d sequence from Gut_metagenome_contig13123000268.

SEQ ID NO: 33 is an exemplary Cas13d sequence from Gut_metagenome_contig5294000434.

SEQ ID NO: 34 is an exemplary Cas13d sequence from Gut_metagenome_contig6415000192.

SEQ ID NO: 35 is an exemplary Cas13d sequence from Gut_metagenome_contig6144000300.

SEQ ID NO: 36 is an exemplary Cas13d sequence from Gut_metagenome_contig9118000041.

SEQ ID NO: 37 is an exemplary Cas13d sequence from Activated_sludge_metagenome_transcript_124486.

SEQ ID NO: 38 is an exemplary Cas13d sequence from Gut_metagenome_contig1322000437.

SEQ ID NO: 39 is an exemplary Cas13d sequence from Gut_metagenome_contig4582000531.

SEQ ID NO: 40 is an exemplary Cas13d sequence from Gut_metagenome_contig9190000283.

SEQ ID NO: 41 is an exemplary Cas13d sequence from Gut_metagenome_contigl709000510.

SEQ ID NO: 42 is an exemplary Cas13d sequence from M24_(LSQX01212483_Anaerobic_digester_metagenome) with a HEPN domain.

SEQ ID NO: 43 is an exemplary Cas13d sequence from Gut_metagenome_contig3833000494.

SEQ ID NO: 44 is an exemplary Cas13d sequence from Activated_sludge_metagenome_transcript_117355.

SEQ ID NO: 45 is an exemplary Cas13d sequence from Gut_metagenome_contigl061000330.

SEQ ID NO: 46 is an exemplary Cas13d sequence from Gut_metagenome_contig338000322 from sheep gut metagenome.

SEQ ID NO: 47 is an exemplary Cas13d sequence from human gut metagenome.

SEQ ID NO: 48 is an exemplary Cas13d sequence from Gut_metagenome_contig9530000097.

SEQ ID NO: 49 is an exemplary Cas13d sequence from Gut_metagenome_contigl750000258.

SEQ ID NO: 50 is an exemplary Cas13d sequence from Gut_metagenome_contig5377000274.

SEQ ID NO: 51 is an exemplary Cas13d sequence from gut_metagenome_P19E0k2120140920_c248000089.

SEQ ID NO: 52 is an exemplary Cas13d sequence from Gut_metagenome_contigl400000031.

SEQ ID NO: 53 is an exemplary Cas13d sequence from Gut_metagenome_contig7940000191.

SEQ ID NO: 54 is an exemplary Cas13d sequence from Gut_metagenome_contig6049000251.

SEQ ID NO: 55 is an exemplary Cas13d sequence from Gut_metagenome_contigl137000500.

SEQ ID NO: 56 is an exemplary Cas13d sequence from Gut_metagenome_contig9368000105.

SEQ ID NO: 57 is an exemplary Cas13d sequence from Gut_metagenome_contig546000275.

SEQ ID NO: 58 is an exemplary Cas13d sequence from Gut_metagenome_contig7216000573.

SEQ ID NO: 59 is an exemplary Cas13d sequence from Gut_metagenome_contig4806000409.

SEQ ID NO: 60 is an exemplary Cas13d sequence from Gut_metagenome_contig10762000480.

SEQ ID NO: 61 is an exemplary Cas13d sequence from Gut_metagenome_contig4114000374.

SEQ ID NO: 62 is an exemplary Cas13d sequence from Ruminococcus_flavefaciens_FD1.

SEQ ID NO: 63 is an exemplary Cas13d sequence from Gut_metagenome_contig7093000170.

SEQ ID NO: 64 is an exemplary Cas13d sequence from Gut_metagenome_contigl1113000384.

SEQ ID NO: 65 is an exemplary Cas13d sequence from Gut_metagenome_contig6403000259.

SEQ ID NO: 66 is an exemplary Cas13d sequence from Gut_metagenome_contig6193000124.

SEQ ID NO: 67 is an exemplary Cas13d sequence from Gut_metagenome_contig721000619.

SEQ ID NO: 68 is an exemplary Cas13d sequence from Gut_metagenome_contigl666000270.

SEQ ID NO: 69 is an exemplary Cas13d sequence from Gut_metagenome_contig2002000411.

SEQ ID NO: 70 is an exemplary Cas13d sequence from Ruminococcus_albus.

SEQ ID NO: 71 is an exemplary Cas13d sequence from Gut_metagenome_contig13552000311.

SEQ ID NO: 72 is an exemplary Cas13d sequence from Gut_metagenome_contig10037000527.

SEQ ID NO: 73 is an exemplary Cas13d sequence from Gut_metagenome_contig238000329.

SEQ ID NO: 74 is an exemplary Cas13d sequence from Gut_metagenome_contig2643000492.

SEQ ID NO: 75 is an exemplary Cas13d sequence from Gut_metagenome_contig874000057.

SEQ ID NO: 76 is an exemplary Cas13d sequence from Gut_metagenome_contig4781000489.

SEQ ID NO: 77 is an exemplary Cas13d sequence from Gut_metagenome_contigl2144000352.

SEQ ID NO: 78 is an exemplary Cas13d sequence from Gut_metagenome_contig5590000448.

SEQ ID NO: 79 is an exemplary Cas13d sequence from Gut_metagenome_contig9269000031.

SEQ ID NO: 80 is an exemplary Cas13d sequence from Gut_metagenome_contig8537000520.

SEQ ID NO: 81 is an exemplary Cas13d sequence from Gut_metagenome_contigl845000130.

SEQ ID NO: 82 is an exemplary Cas13d sequence from gut_metagenome_P13E0k2120140920_c3000072.

SEQ ID NO: 83 is an exemplary Cas13d sequence from gut_metagenome_P1 E0k2120140920_cI000078.

SEQ ID NO: 84 is an exemplary Cas13d sequence from Gut_metagenome_contigl2990000099.

SEQ ID NO: 85 is an exemplary Cas13d sequence from Gut_metagenome_contig525000349.

SEQ ID NO: 86 is an exemplary Cas13d sequence from Gut_metagenome_contig7229000302.

SEQ ID NO: 87 is an exemplary Cas13d sequence from Gut_metagenome_contig3227000343.

SEQ ID NO: 88 is an exemplary Cas13d sequence from Gut_metagenome_contig7030000469.

SEQ ID NO: 89 is an exemplary Cas13d sequence from Gut_metagenome_contig5149000068.

SEQ ID NO: 90 is an exemplary Cas13d sequence from Gut_metagenome_contig400200045.

SEQ ID NO: 91 is an exemplary Cas13d sequence from Gut_metagenome_contig10420000446.

SEQ ID NO: 92 is an exemplary Cas13d sequence from new_flavefaciens_strain_XPD3002 (CasRx).

SEQ ID NO: 93 is an exemplary Cas13d sequence from M26_Gut_metagenome_contig698000307.

SEQ ID NO: 94 is an exemplary Cas13d sequence from M36_Uncultured_Eubacterium_sp_TS28_c40956.

SEQ ID NO: 95 is an exemplary Cas13d sequence from M12_gut_metagenome_P25Ck2120140920_c134000066.

SEQ ID NO: 96 is an exemplary Cas13d sequence from human gut metagenome.

SEQ ID NO: 97 is an exemplary Cas13d sequence from MlO_gut_metagenome_P25C90k2120 1 40920_c2800004 1.

SEQ ID NO: 98 is an exemplary Cas13d sequence from 30 Ml I_gut_metagenome_P25C7k2120140920_c4078000105.

SEQ ID NO: 99 is an exemplary Cas13d sequence from gut_metagenome_P25C0k2120140920_c32000045.

SEQ ID NO: 100 is an exemplary Cas13d sequence from M13_gut_metagenome_P23C7k2120140920 c3000067.

SEQ ID NO: 101 is an exemplary Cas13d sequence from M5_gut_metagenome_P8E90k2120140920.

SEQ ID NO: 102 is an exemplary Cas13d sequence from M21_gut_metagenome_P8E0k2120140920.

SEQ ID NO: 103 is an exemplary Cas13d sequence from M7_gut_metagenome_P38C7k2120 1 40920_c484 1000003.

SEQ ID NO: 104 is an exemplary Cas13d sequence from Ruminococcus_bicirculans.

SEQ ID NO: 105 is an exemplary Cas13d sequence.

SEQ ID NO: 106 is an exemplary Cas13d consensus sequence.

SEQ ID NO: 107 is an exemplary Cas13d sequence from M18_gut_metagenome_P22EOk2120140920_c3395000078.

SEQ ID NO: 108 is an exemplary Cas13d sequence from M17_gut_metagenome_P22E90k2120140920_c114.

SEQ ID NO: 109 is an exemplary Cas13d sequence from Ruminococcus_sp_CAG57.

SEQ ID NO: 110 is an exemplary Cas13d sequence from gut_metagenome_Pl 1E90k2120140920_c43000123.

SEQ ID NO: 111 is an exemplary Cas13d sequence from M6_gut_metagenome_P13E90k2120 1 40920_c7000009.

SEQ ID NO: 112 is an exemplary Cas13d sequence from M19_gut_metagenome_Pl 7E90k2120140920.

SEQ ID NO: 113 is an exemplary Cas13d sequence from gut_metagenome_Pl7E0k2120140920,_c87000043.

SEQ ID NO: 114 is an exemplary human codon optimized Eubacterium siraeum Cas13d nucleic acid sequence.

SEQ ID NO: 115 is an exemplary human codon optimized Eubacterium siraeum Cas13d nucleic acid sequence with a mutant HEPN domain.

SEQ ID NO: 116 is an exemplary human codon-optimized Eubacterium siraeum Cas13d nucleic acid sequence with N-terminal NLS.

SEQ ID NO: 117 is an exemplary human codon-optimized Eubacterium siraeum Cas13d nucleic acid sequence with N- and C-terminal NLS tags.

SEQ ID NO: 118 is an exemplary human codon-optimized uncultured Ruminococcus sp. Cas13d 30 nucleic acid sequence.

SEQ ID NO: 119 is an exemplary human codon-optimized uncultured Ruminococcus sp. Cas13d nucleic acid sequence with a mutant HEPN domain.

SEQ ID NO: 120 is an exemplary human codon-optimized uncultured Ruminococcus sp. Cas13d nucleic acid sequence with N-terminal NLS.

SEQ ID NO: 121 is an exemplary human codon-optimized uncultured Ruminococcus sp. Cas13d nucleic acid sequence with N- and C-terminal NLS tags.

SEQ ID NO: 122 is an exemplary human codon-optimized uncultured Ruminococcus flavefaciens FDl Cas13d nucleic acid sequence.

SEQ ID NO: 123 is an exemplary human codon-optimized uncultured Ruminococcus flavefaciens FDl Cas13d nucleic acid sequence with mutated HEPN domain.

SEQ ID NO: 124 is an exemplary Cas13d nucleic acid sequence from Ruminococcus bicirculans.

SEQ ID NO: 125 is an exemplary Cas13d nucleic acid sequence from Eubacterium siraeum.

SEQ ID NO: 126 is an exemplary Cas13d nucleic acid sequence from Ruminococcus flavefaciens FD1.

SEQ ID NO: 127 is an exemplary Cas13d nucleic acid sequence from Ruminococcus albus.

SEQ ID NO: 128 is an exemplary Cas13d nucleic acid sequence from Ruminococcus flavefaciens XPD.

SEQ ID NO: 129 is an exemplary consensus DR nucleic acid sequence for E. siraeum Cas13d.

SEQ ID NO: 130 is an exemplary consensus DR nucleic acid sequence for Rum. Sp. Cas13d.

SEQ ID NO: 131 is an exemplary consensus DR nucleic acid sequence for Rum. Flavefaciens strain XPD3002 Cas13d (CasRx).

SEQ ID NOS: 132-137 are exemplary consensus DR nucleic acid sequences.

SEQ ID NO: 138 is an exemplary 50% consensus sequence for seven full-length Cas13d orthologues.

SEQ ID NO: 139 is an exemplary Cas13d nucleic acid sequence from Gut metagenome PlEO.

SEQ ID NO: 140 is an exemplary Cas13d nucleic acid sequence from Anaerobic digester.

SEQ ID NO: 141 is an exemplary Cas13d nucleic acid sequence from Ruminococcus sp. CAG:57.

SEQ ID NO: 142 is an exemplary human codon-optimized uncultured Gut metagenome PlEO Cas13d nucleic acid sequence.

SEQ ID NO: 143 is an exemplary human codon-optimized Anaerobic Digester Cas13d nucleic acid sequence.

SEQ ID NO: 144 is an exemplary human codon-optimized Ruminococcus flavefaciens XPD Cas13d nucleic acid sequence.

SEQ ID NO: 145 is an exemplary human codon-optimized Ruminococcus albus Cas13d nucleic acid sequence.

SEQ ID NO: 146 is an exemplary processing of the Ruminococcus sp. CAG:57 CRISPR array.

SEQ ID NO: 147 is an exemplary Cas13d protein sequence from contig emb |OBVH01003037.1, human gut metagenome sequence (also found in WGS contigs emb |OBXZ01000094.1| and emb |OBJFO1000033.1.

SEQ ID NO: 148 is an exemplary consensus DR nucleic acid sequence (goes with SEQ ID NO:147).

SEQ ID NO: 149 is an exemplary Cas13d protein sequence from contig tpg |DBYI01000091.1| (Uncultivated Ruminococcus flavefaciens UBA1190 assembled from bovine gut metagenome).

SEQ ID NOS: 150-152 are exemplary consensus DR nucleic acid sequences (goes with SEQ ID NO: 149).

SEQ ID NO: 153 is an exemplary Cas13d protein sequence from contig tpg |DJXD01000002.1| (uncultivated Ruminococcus assembly, UBA7013, from sheep gutmetagenome).

SEQ ID NO: 154 is an exemplary consensus DR nucleic acid sequence (goes with SEQ ID NO: 153).

SEQ ID NO: 155 is an exemplary Cas13d protein sequence from contig OGZC01000639.1 (human gut metagenome assembly).

SEQ ID NOS: 156-177 are exemplary consensus DR nucleic acid sequences (goes with SEQ ID NO: 155).

SEQ ID NO: 158 is an exemplary Cas13d protein sequence from contig emb |OHBM01000764.1 (human gut metagenome assembly).

SEQ ID NO: 159 is an exemplary consensus DR nucleic acid sequence (goes with SEQ ID NO:158).

SEQ ID NO: 160 is an exemplary Cas13d protein sequence from contig emb |0HCP01000044.1 (human gut metagenome assembly).

SEQ ID NO: 161 is an exemplary consensus DR nucleic acid sequence (goes with SEQ ID NO: 160).

SEQ ID NO: 162 is an exemplary Cas13d protein sequence from contig embl0GDF01008514.1| (human gut metagenome assembly).

SEQ ID NO: 163 is an exemplary consensus DR nucleic acid sequence (goes with SEQ ID NO: 162).

SEQ ID NO: 164 is an exemplary Cas13d protein sequence from contig emb |0GPN01002610.1 (human gut metagenome assembly).

SEQ ID NO: 165 is an exemplary consensus DR nucleic acid sequence (goes with SEQ ID NO: 164).

SEQ ID NO: 166 is an exemplary Cas13d protein sequence from contig NFIR01000008. 1 (Eubacterium sp. An3, from chicken gut metagenome).

SEQ ID NO: 167 is an exemplary consensus DR nucleic acid sequence (goes with SEQ ID NO: 166).

SEQ ID NO: 168 is an exemplary Cas13d protein sequence from contig NFLV01000009.1 (Eubacterium sp. An11 from chicken gut metagenome).

SEQ ID NO: 169 is an exemplary consensus DR nucleic acid sequence (goes with SEQ ID NO: 168).

SEQ ID NOS: 171-174 are an exemplary Cas13d motif sequences.

SEQ ID NO: 175 is an exemplary Cas13d protein sequence from contig OJMM01002900 human gut metagenome sequence.

SEQ ID NO: 176 is an exemplary consensus DR nucleic acid sequence (goes with SEQ ID NO: 175).

SEQ ID NO: 177 is an exemplary Cas13d protein sequence from contig ODAI011611274.1 gut metagenome sequence.

SEQ ID NO: 178 is an exemplary consensus DR nucleic acid sequence (goes with SEQ ID NO: 177).

SEQ ID NO: 179 is an exemplary Cas13d protein sequence from contig OIZX01000427.1.

SEQ ID NO: 180 is an exemplary consensus DR nucleic acid sequence (goes with SEQ ID NO:179).

SEQ ID NO: 181 is an exemplary Cas13d protein sequence from contig emb |OCVV012889144.1|.

SEQ ID NO: 182 is an exemplary consensus DR nucleic acid sequence (goes with SEQ ID NO: 181).

SEQ ID NO: 183 is an exemplary Cas13d protein sequence from contig OCTW011587266.1

SEQ ID NO: 184 is an exemplary consensus DR nucleic acid sequence (goes with SEQ ID NO: 183).

SEQ ID NO: 185 is an exemplary Cas13d protein sequence from contig emb |OGNFO 1009141.1.

SEQ ID NO: 186 is an exemplary consensus DR nucleic acid sequence (goes with SEQ ID NO: 185).

SEQ ID NO: 187 is an exemplary Cas13d protein sequence from contig emb |OIEN01002196.1.

SEQ ID NO: 188 is an exemplary consensus DR nucleic acid sequence (goes with SEQ ID NO: 187).

SEQ ID NO: 189 is an exemplary Cas13d protein sequence from contig e-k87_11092736.

SEQ ID NOS: 190-193 are exemplary consensus DR nucleic acid sequences (goes with SEQ ID NO: 189).

SEQ ID NO: 194 is an exemplary Cas13d sequence from Gut_metagenome_contig6893000291.

SEQ ID NOS: 195-197 are exemplary Cas13d motif sequences.

SEQ ID NO: 198 is an exemplary Cas13d protein sequence from Ga0224415_10007274.

SEQ ID NO: 199 is an exemplary consensus DR nucleic acid sequence (goes with SEQ ID NO: 198).

SEQ ID NO: 200 is an exemplary Cas13d protein sequence from EMG_10003641.

SEQ ID NO: 202 is an exemplary Cas13d protein sequence from Ga0129306_1000735.

SEQ ID NO: 201 is an exemplary consensus DR nucleic acid sequence (goes with SEQ ID NO: 200).

SEQ ID NO: 202 is an exemplary Cas13d protein sequence from Ga0129306_1000735.

SEQ ID NO: 203 is an exemplary consensus DR nucleic acid sequence (goes with SEQ ID NO: 203

SEQ ID NO: 204 is an exemplary Cas13d protein sequence from GaO129317_1 008067.

SEQ ID NO: 205 is an exemplary consensus DR nucleic acid sequence (goes with SEQ ID NO: 204).

SEQ ID NO: 206 is an exemplary Cas13d protein sequence from Ga0224415_10048792.

SEQ ID NO: 207 is an exemplary consensus DR nucleic acid sequence (goes with SEQ ID NO: 206).

SEQ ID NO: 208 is an exemplary Cas13d protein sequence from 160582958_gene49834.

SEQ ID NO: 209 is an exemplary consensus DR nucleic acid sequence (goes with SEQ ID NO: 208).

SEQ ID NO: 210 is an exemplary Cas13d protein sequence from 250twins_35838_GL0110300.

SEQ ID NO: 211 is an exemplary consensus DR nucleic acid sequence (goes with SEQ ID NO: 210).

SEQ ID NO: 212 is an exemplary Cas13d protein sequence from 250twins_36050_GLOI58985.

SEQ ID NO: 213 is an exemplary consensus DR nucleic acid sequence (goes with SEQ ID NO: 212).

SEQ ID NO: 214 is an exemplary Cas13d protein sequence from 31009_GL0034153.

SEQ ID NO: 215 is an exemplary consensus DR nucleic acid sequence (goes with SEQ ID NO: 214).

SEQ ID NO: 216 is an exemplary Cas13d protein sequence from 530373_GL0023589.

SEQ ID NO: 217 is an exemplary consensus DR nucleic acid sequence (goes with SEQ ID NO: 216).

SEQ ID NO: 218 is an exemplary Cas13d protein sequence from BMZ-1 1B_GL0037771.

SEQ ID NO: 219 is an exemplary consensus DR nucleic acid sequence (goes with SEQ ID NO: 218).

SEQ ID NO: 220 is an exemplary Cas13d protein sequence from BMZ-1 1B_GL0037915.

SEQ ID NO: 221 is an exemplary consensus DR nucleic acid sequence (goes with SEQ ID NO: 220).

SEQ ID NO: 222 is an exemplary Cas13d protein sequence from BMZ-1 1B_GL00696 17.

SEQ ID NO: 223 is an exemplary consensus DR nucleic acid sequence (goes with SEQ ID NO: 222).

SEQ ID NO: 224 is an exemplary Cas13d protein sequence from DLF014_GL0011914.

SEQ ID NO: 225 is an exemplary consensus DR nucleic acid sequence (goes with SEQ ID NO: 224).

SEQ ID NO: 226 is an exemplary Cas13d protein sequence from EYZ-362B_GL0088915.

SEQ ID NO: 227-228 are exemplary consensus DR nucleic acid sequences (goes with SEQ ID NO: 226).

SEQ ID NO: 229 is an exemplary Cas13d protein sequence from Ga0099364 10024192.

SEQ ID NO: 230 is an exemplary consensus DR nucleic acid sequence (goes with SEQ ID NO: 229).

SEQ ID NO: 231 is an exemplary Cas13d protein sequence from Ga0187910_10006931.

SEQ ID NO: 232 is an exemplary consensus DR nucleic acid sequence (goes with SEQ ID NO: 231).

SEQ ID NO: 233 is an exemplary Cas13d protein sequence from Ga0187910_10015336.

SEQ ID NO: 234 is an exemplary consensus DR nucleic acid sequence (goes with SEQ ID NO: 233).

SEQ ID NO: 235 is an exemplary Cas13d protein sequence from Ga0187910_10040531.

SEQ ID NO: 236 is an exemplary consensus DR nucleic acid sequence (goes with SEQ ID NO: 23).

SEQ ID NO: 237 is an exemplary Cas13d protein sequence from Ga0187911_10069260.

SEQ ID NO: 238 is an exemplary consensus DR nucleic acid sequence (goes with SEQ ID NO: 237).

SEQ ID NO: 239 is an exemplary Cas13d protein sequence from MH0288_GL0082219.

SEQ ID NO: 240 is an exemplary consensus DR nucleic acid sequence (goes with SEQ ID NO: 239).

SEQ ID NO: 241 is an exemplary Cas13d protein sequence from O2.UC29-0_GL0096317.

SEQ ID NO: 242 is an exemplary consensus DR nucleic acid sequence (goes with SEQ ID NO: 241).

SEQ ID NO: 243 is an exemplary Cas13d protein sequence from PIG-014_GL0226364.

SEQ ID NO: 244 is an exemplary consensus DR nucleic acid sequence (goes with SEQ ID NO: 243).

SEQ ID NO: 245 is an exemplary Cas13d protein sequence from PIG-018_GL0023397.

SEQ ID NO: 246 is an exemplary consensus DR nucleic acid sequence (goes with SEQ ID NO: 245).

SEQ ID NO: 247 is an exemplary Cas13d protein sequence from PIG-025_GL0099734.

SEQ ID NO: 248 is an exemplary consensus DR nucleic acid sequence (goes with SEQ ID NO: 247).

SEQ ID NO: 249 is an exemplary Cas13d protein sequence from PIG-028_GL0185479.

SEQ ID NO: 250 is an exemplary consensus DR nucleic acid sequence (goes with SEQ ID NO: 249).

SEQ ID NO: 251 is an exemplary Cas13d protein sequence from -Ga0224422_10645759.

SEQ ID NO: 252 is an exemplary consensus DR nucleic acid sequence (goes with SEQ ID NO: 251).

SEQ ID NO: 253 is an exemplary Cas13d protein sequence from ODAI chimera.

SEQ ID NO: 254 is an exemplary consensus DR nucleic acid sequence (goes with SEQ ID NO: 253).

SEQ ID NO: 255 is an HEPN motif.

SEQ ID NOs: 256 and 257 are exemplary Cas13d nuclear localization signal amino acid and nucleic acid sequences, respectively.

SEQ ID NOs: 258 and 260 are exemplary SV40 large T antigen nuclear localization signal amino acid and nucleic acid sequences, respectively.

SEQ ID NO: 259 is a dCas9 target sequence.

SEQ ID NO: 261 is an artificial Eubacterium siraeum nCasl array targeting ccdB.

SEQ ID NO: 262 is a full 36 nt direct repeat.

SEQ ID NOs: 263-266 are spacer sequences.

SEQ ID NO: 267 is an artificial uncultured Ruminoccus sp. nCasl array targeting ccdB.

SEQ ID NO: 268 is a full 36 nt direct repeat.

SEQ ID NOs: 269-272 are spacer sequences.

SEQ ID NO: 273 is a ccdB target RNA sequence.

SEQ ID NOs: 274-277 are spacer sequences.

SEQ ID NO: 278 is a mutated Cas13d sequence, NLS-Ga_0531(trunc)-NLS-HA. This mutant has a deletion of the non-conserved N-terminus.

SEQ ID NO: 279 is a mutated Cas13d sequence, NES-Ga_0531(trunc)-NES-HA. This mutant has a deletion of the non-conserved N-terminus.

SEQ ID NO: 280 is a full-length Cas13d sequence, NLS-RfxCas13d-NLS-HA.

SEQ ID NO: 281 is a mutated Cas13d sequence, NLS-RfxCas13d(del5)-NLS-HA. This mutant has a deletion of amino acids 558-587.

SEQ ID NO: 282 is a mutated Cas13d sequence, NLS-RfxCas13d(del5.12)-NLS-HA. This mutant has a deletion of amino acids 558-587 and 953-966.

SEQ ID NO: 283 is a mutated Cas13d sequence, NLS-RfxCas13d(del5.13)-NLS-HA. This mutant has a deletion of amino acids 376-392 and 558-587.

SEQ ID NO: 284 is a mutated Cas13d sequence, NLS-RfxCas13d(del5.12+5.13)-NLS-HA. This mutant has a deletion of amino acids 376-392, 558-587, and 953-966.

SEQ ID NO: 285 is a mutated Cas13d sequence, NLS-RfxCas13d(dell3)-NLS-HA. This mutant has a deletion of amino acids 376-392.

SEQ ID NO: 286 is an effector sequence used to edit expression of ADAR2. Amino acids 1 to 969 are dRfxCas13, aa 970 to 991 are an NLS sequence, and amino acids 992 to 1378 are ADAR2DD.

SEQ ID NO: 287 is an exemplary HIV NES protein sequence.

SEQ ID NOS: 288-291 are exemplary Cas13d motif sequences.

SEQ ID NO: 292 is Cas13d ortholog sequence MH_4866.

SEQ ID NO: 293 is an exemplary Cas13d protein sequence from 037_-_emblOIZA01000315.11

SEQ ID NO: 294 is an exemplary Cas13d protein sequence from PIG-022 GL002635 1.

SEQ ID NO: 295 is an exemplary Cas13d protein sequence from PIG-046_GL0077813.

SEQ ID NO: 296 is an exemplary Cas13d protein sequence from pig_chimera.

SEQ ID NO: 297 is an exemplary nuclease-inactive or dead Cas13d (dCas13d) protein sequence from Ruminococcus flavefaciens XPD3002 (CasRx)

SEQ ID NO: 298 is an exemplary Cas13d protein sequence.

SEQ ID NO: 299 is an exemplary Cas13d protein sequence from (contig tpg|DJXD01000002.1|; uncultivated Ruminococcus assembly, UBA7013, from sheep gut metagenome).

SEQ ID NO: 300 is an exemplary Cas13d direct repeat nucleotide sequence from Cas13d (contig tpg|DJXD01000002.1|; uncultivated Ruminococcus assembly, UBA7013, from sheep gut metagenome (goes with SEQ ID NO: 299).

SEQ ID NO: 301 is an exemplary Cas13d protein contig emb|OBLI01020244.

Yan et al. (2018) Mol Cell. 70(2):327-339 (doi: 10.1016/j.molcel.2018.02.2018) and Konermann et al. (2018) Cell 173(3):665-676 (doi: 10.1016/j.cell/2018.02.033) have described Cas13d proteins and both of which are incorporated by reference herein in their entireties. Also see WO Publication Nos. WO2018/183403 (CasM, which is Cas13d) and WO2019/006471 (Cas13d), which are incorporated herein by reference in their entirety.

SEQ ID NO: 467 is an exemplary CasM protein from Eubacterium siraeum.

SEQ ID NO: 468 is an exemplary CasM protein from Ruminococcus sp., isolate 2789STDY5834971.

SEQ ID NO: 469 is an exemplary CasM protein from Ruminococcus bicirculans.

SEQ ID NO: 470 is an exemplary CasM protein from Ruminococcus sp., isolate 2789STDY5608892.

SEQ ID NO: 471 is an exemplary CasM protein from Ruminococcus sp. CAG:57.

SEQ ID NO: 472 is an exemplary CasM protein from Ruminococcus flavefaciens FD-1.

SEQ ID NO: 473 is an exemplary CasM protein from Ruminococcus albus strain KH2T6.

SEQ ID NO: 474 is an exemplary CasM protein from Ruminococcus flavefaciens strain XPD3002.

SEQ ID NO: 475 is an exemplary CasM protein from Ruminococcus sp., isolate 2789STDY5834894.

SEQ ID NO: 476 is an exemplary RtcB homolog.

SEQ ID NO: 477 is an exemplary WYL from Eubacterium siraeum+C-terminal NLS.

SEQ ID NO: 478 is an exemplary WYL from Ruminococcus sp. isolate 2789STDY5834971+C-term NLS.

SEQ ID NO: 479 is an exemplary WYL from Ruminococcus bicirculans+C-term NLS.

SEQ ID NO: 480 is an exemplary WYL from Ruminococcus sp. isolate 2789STDY5608892+C-term NLS.

SEQ ID NO: 481 is an exemplary WYL from Ruminococcus sp. CAG:57+C-term NLS.

SEQ ID NO: 482 is an exemplary WYL from Ruminococcus flavefaciens FD-1+C-term NLS.

SEQ ID NO: 483 is an exemplary WYL from Ruminococcus albus strain KH2T6+C-term NLS.

SEQ ID NO: 484 is an exemplary WYL from Ruminococcus flavefaciens strain XPD3002+C-term NLS.

SEQ ID NO: 485 is an exemplary RtcB from Eubacterium siraeum+C-term NLS.

Exemplary wild type Cas13d proteins of the disclosure may comprise or consist of the amino acid sequence SEQ ID NO: 92 or SEQ ID NO: 298 (Cas13d protein also known as CasRx).

An exemplary direct repeat sequence of Ruminococcus flavefaciens XPD3002 Cas13d (CasRx) comprises the nucleic acid sequence:

(SEQ ID NO: 461)
AACCCCTACCAACTGGTCGGGGTTTGAAAC.

### Therapeutic Replacement Genes (Corresponding Disease/Disorder to be Treated)

Compositions comprising therapeutic replacement genes disclosed herein include any effective gain-or-loss-of-function gene replacement therapies. Exemplary therapeutic replacement genes (corresponding diseases) include, without limitation, genes (diseases/disorders) such as rhodopsin (Retinitis Pigmentosa), PRPF3—Pre-mRNA Splicing Factor 3 (autosomal dominant Retinitis Pigmentosa), PRPF31 (autosomal dominant Retinitis Pigmentosa), GRN (Frontotemporal dementia (FTD)), SOD1 (ALS), PMP22 (Charcot Marie Tooth Disease), PABPN1 (Oculopharangeal Muscular Dystrophy), KCNQ4 (Hearing Loss), CLRN1 (Usher Syndrome), APOE2 (Alzheimer's Disease), APOE4 (Alzheimer's Disease), BEST1 (Eye Disease), MYBPC3 (Familial Cardiomyopathy), TNNT2 (Familial Cardiomyopathy), and TNNI3 (Familial Cardiomyopathy).

In some embodiments, therapeutic replacement genes are codon optimized. In some embodiments, the codons relevant to the target site are not codon optimized. In some embodiments, the RNA-targeting proteins of the disclosure ensure cleavage of the mutant allele but not cleavage of the transgene or therapeutic replacement gene.

Exemplary therapeutic replacement genes and corresponding sequences include, without limitation, the following:

### Rhodopsin (Human RHO)

Exemplary therapeutic replacement genes may comprise or consist of the amino acid sequence of Rhodopsin:

(SEQ ID NO: 302)
MNGTEGPNFYVPFSNATGVVRSPFEYPQYYLAEPWQFSMLAAYMFLLIVL
GFPINFLTLYVTVQHKKLRTPLNYILLNLAVADLFMVLGGFTSTLYTSLH
GYFVFGPTGCNLEGFFATLGGEIALWSLVVLAIERYVVVCKPMSNFRFGE
NHAIMGVAFTWVMALACAAPPLAGWSRYIPEGLQCSCGIDYYTLKPEVNN
ESFVIYMFVVHFFTIPMIIIFFCYGQLVFTVKFAAAQQQESATTQKAEKE
VTRMVIIMVIAFLICWVPYASVAFYIFTHQGSNFGPIFMTIPAFFAKSAA
IYNPVIYIMMNKQFRNCMLTTICCGKNPLGDDEASATVSKTETSQVAPA.

### Super Oxide Dismutase 1 (SOD1)

Exemplary therapeutic replacement genes may comprise or consist of the amino acid sequence of Super Oxide Dismutase 1:

(SEQ ID NO: 303)
MATKAVCVLKGDGPVQGIINFEQKESNGPVKVWGSIKGLTEGLHGFHHVH
EFGDNTAGCTSAGPHFNPLSRKHGGPKDEERHVGDLGNVTADKDGVADVS
IEDSVISGDHCIIGRTLVVHEKADDLGKGGNEESTKTGNAGSRLACCVIG
IAQ.

### Peripheral Myelin Protein 22 (PMP22)

Exemplary therapeutic replacement genes may comprise or consist of the amino acid sequence of Peripheral Myelin Protein 22:

(SEQ ID NO: 304)
MLLLLLSIINTLHVAVLVLLFVSTIVSQWIVGNGHATDLWQNCSTSSSGN
VHHCFSSSPNEWLQSVQATMILSIIFSILSLFLFFCQLFTLTKGGRFYIT
GIFQILAGLCVMSAAAIYTVRHPEWHLNSDYSYGFAYILAWVAFPLALLS
GVIYVILRKRE.

### Poly(A) Binding Protein Nuclear 1 (PABPN1)

Exemplary therapeutic replacement genes may comprise or consist of the amino acid sequence of Poly(A) Binding Protein Nuclear 1:

(SEQ ID NO: 305)
MAAAAAAAAAAGAAGGRGSGPGRRRHLVPGAGGEAGEGAPGGAGDYGNGL
ESEELEPEELLLFPEPEPEPEEEPPRPRAPPGAPGPGPGSGAPGSQEEEE
EPGLVEGDPGDGAIEDPELEAIKARVREMEEEAEKLKELQNEVEKOMNMS
PPPGNAGPVIMSIEEKMEADARSIYVGNVDYGATAEELEAHFHGCGSVNR
VTILCDKFSGHPKGFAYIEFSDKESVRTSLALDESLFRGRQIKVIPKRTN
RPGISTTDRGFPRARYRARTTNYNSSRSRFYSGFNSRPRGRVYRGRARAT
SWYSPY.

### Potassium Voltage-Gated Channel Subfamily Q Member 4 (KCNQ4)

Exemplary therapeutic replacement genes may comprise or consist of the amino acid sequence of Potassium Voltage-Gated Channel Subfamily Q Member 4:

(SEQ ID NO: 306)
MAEAPPRRLGLGPPPGDAPRAELVALTAVQSEQGEAGGGGSPRRLGLLGS
PLPPGAPLPGPGSGSGSACGQRSSAAHKRYRRLQNWVYNVLERPRGWAFV
YHVFIFLLVFSCLVLSVLSTIQEHQELANECLLILEFVMIVVFGLEYIVR
VWSAGCCCRYRGWQGRFRFARKPFCVIDFIVFVASVAVIAAGTQGNIFAT
SALRSMRFLQILRMVRMDRRGGTWKLLGSVVYAHSKELITAWYIGFLVLI
FASFLVYLAEKDANSDFSSYADSLWWGTITLTTIGYGDKTPHTWLGRVLA
AGFALLGISFFALPAGILGSGFALKVQEQHRQKHFEKRRMPAANLIQAAW
RLYSTDMSRAYLTATWYYYDSILPSFRELALLFEHVQRARNGGLRPLEVR
RAPVPDGAPSRYPPVATCHRPGSTSFCPGESSRMGIKDRIRMGSSQRRTG
PSKQHLAPPTMPTSPSSEQVGEATSPTKVQKSWSFNDRTRFRASLRLKPR
TSAEDAPSEEVAEEKSYQCELTVDDIMPAVKTVIRSIRILKFLVAKRKFK
ETLRPYDVKDVIEQYSAGHLDMLGRIKSLQTRVDQIVGRGPGDRKAREKG
DKGPSDAEVVDEISMMGRVVKVEKQVQSIEHKLDLLLGFYSRCLRSGTSA
SLGAVQVPLFDPDITSDYHSPVDHEDISVSAQTLSISRSVSTNMD.

### Clarin 1 (CLRN1)

Exemplary therapeutic replacement genes may comprise or consist of the amino acid sequence of Clarin 1:

(SEQ ID NO: 307)
MPSQQKKIIFCMAGVFSFACALGVVTALGTPLWIKATVLCKTGALLVNAS
GQELDKFMGEMQYGLFHGEGVRQCGLGARPFRFSFFPDLLKAIPVSIHVN
VILFSAILIVLTMVGTAFFMYNAFGKPFETLHGPLGLYLLSFISGSCGCL
VMILFASEVKIHEILSEKIANYKEGTYVYKTQSEKYTTSFWVIFFCFFVH
FLNGLLIRLAGFQFPFAKSKDAETTNVAADLM.

### Apolipoprotein 2 (APOE2)

Exemplary therapeutic replacement genes may comprise or consist of the amino acid sequence of Apolipoprotein 2.

(SEQ ID NO: 308)
MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVCGRLVQYRGEVQANILGQSTE
ELRVRLASHLRKLRKRLLRDADDLQKCLAVYQAGAREGAERGLSAIRERL
GPLVEQGRVRAATVGSLAGQPLQERAQAWGERLRARMEEMGSRTRDRLDE
VKEQVAEVRAKLEEQAQQIRLQAEAFQARLKSWFEPLVEDMQRQWAGLVE
KVQAAVGTSAAPVPSDNH.

### Apolipoprotein 4 (APOE4)

Exemplary therapeutic replacement genes may comprise or consist of the amino acid sequence of Apolipoprotein 4:

(SEQ ID NO: 309)
MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVRGRLVQYRGEVQAMLGQSTEE
LRVRLASHLRKLRKRLLRDADDLQKRLAVYQAGAREGAERGLSAIRERLG
PLVEQGRVRAATVGSLAGQPLQERAQAWGERLRARMEEMGSRTRDRLDEV
KEQVAEVRAKLEEQAQQIRLQAEAFQARLKSWFEPLVEDMQRQWAGLVEK
VQAAVGTSAAPVPSDNH.

### Bestrophin-1 (BEST1)

Exemplary therapeutic replacement genes may comprise or consist of the amino acid sequence of Bestrophin-1:

(SEQ ID NO: 310)
MTITYTSQVANARLGSFSRLLLCWRGSIYKLLYGEFLIFLLCYYIIRFIY
RLALTEEQQLMFEKLTLYCDSYIQLIPISFVLGFYVTLVVTRWWNQYENL
PWPDRLMSLVSGFVEGKDEQGRLLRRTLIRYANLGNVLILRSVSTAVYKR
FPSAQHLVQAGFMTPAEHKQLEKLSLPHNMFWVPWVWFANLSMKAWLGGR
IRDPILLQSLLNEMNTLRTQCGHLYAYDWISIPLVYTQVVTVAVYSEELT
CLVGRQFLNPAKAYPGHELDLVVPVFTFLQFFFYVGWLKVAEQLINPFGE
DDDDFETNWIVDRNLQVSLLAVDEMHQDLPRMEPDMYWNKPEPQPPYTAA
SAQFRRASFMGSTENTSLNKEEMEFQPNQEDEEDAHAGIIGRFLGLQSHD
HHPPRANSRTKLLWPKRESLLHEGLPKNHKAAKQNVRGQEDNKAWKLKAV
DAFKSAPLYQRPGYYSAPQTPLSPTPMFFPLEPSAPSKLHSVTGIDTKDK
SLKTVSSGAKKSFELLSESDGALMEHPEVSQVRRKTVEFNLTDMPEIPEN
HLKEPLEQSPTNIHTTLKDHMDPYWALENRDEAHS.

### Cardiac Myosin-Binding Protein-C(MYBPC3)

Exemplary therapeutic replacement genes may comprise or consist of the amino acid sequence of Cardiac Myosin-Binding Protein-C:

(SEQ ID NO: 311)
MPEPGKKPVSAFSKKPRSVEVAAGSPAVFEAETERAGVKVRWQRGGSDIS
ASNKYGLATEGTRHTLTVREVGPADQGSYAVIAGSSKVKFDLKVIEAEKA
EPMLAPAPAPAEATGAPGEAPAPAAELGESAPSPKGSSSAALNGPTPGAP
DDPIGLFVMRPQDGEVTVGGSITFSARVAGASLLKPPVVKWFKGKWVDLS
SKVGQHLQLHDSYDRASKVYLFELHITDAQPAFTGSYRCEVSTKDKFDCS
NFNLTVHEAMGTGDLDLLSAFRRTSLAGGGRRISDSHEDTGILDFSSLLK
KRDSFRTPRDSKLEAPAEEDVWEILRQAPPSEYERIAFQYGVTDLRGMLK
RLKGMRRDEKKSTAFQKKLEPAYQVSKGHKIRLTVELADHDAEVKWLKNG
QEIQMSGSKYIFESIGAKRTLTISQCSLADDAAYQCVVGGEKCSTELEVK
EPPVLITRPLEDQLVMVGQRVEFECEVSEEGAQVKWLKDGVELTREETFK
YRFKKDGQRHHLIINEAMLEDAGHYALCTSGGQALAELIVQEKKLEVYQS
IADLMVGAKDQAVFKCEVSDENVRGVWLKNGKELVPDSRIKVSHIGRVHK
LTIDDVTPADEADYSFVPEGFACNLSAKLHFMEVKIDFVPRQEPPKIHLD
CPGRIPDTIVVVAGNKLRLDVPISGDPAPTVIWQKAITQGNKAPARPAPD
APEDTGDSDEWVFDKKLLCETEGRVRVETTKDRSIFTVEGAEKEDEGVYT
VTVKNPVGEDQVNLTVKVIDVPDAPAAPKISNVGEDSCTVQWEPPAYDGG
QPILGYILERKKKKSYRWMRLNFDLIQELSHEARRMIEGVVYEMRVYAVN
AIGMSRPSPASQPFMPIGPPSEPTHLAVEDVSDTTVSLKWRPPERVGAGG
LDGYSVEYCPEGCSEWVAALQGLTEHTSILVKDLPTGARLLFRVRAHNMA
GPGAPVTTTEPVTVQEILQRPRLQLPRHLRQTIQKKVGEPVNLLIPFQGK
PRPQVTWTKEGQPLAGEEVSIRNSPTDTILFIRAARRVHSGTYQVTVRIE
NMEDKATLVLQVVDKPSPPQDLRVTDAWGLNVALEWKPPQDVGNTELWGY
TVQKADKKTMEWFTVLEHYRRTHCVVPELIIGNGYYFRVFSQNMVGFSDR
AATTKEPVFIPRPGITYEPPNYKALDFSEAPSFTQPLVNRSVIAGYTAML
CCAVRGSPKPKISWFKNGLDLGEDARFRMFSKQGVLTLEIRKPCPFDGGI
YVCRATNLQGEARCECRLEVRVPQ.

### Cardiac Troponin T2 (TNNT2)

Exemplary therapeutic replacement genes may comprise or consist of the amino acid sequence of Cardiac Troponin T2:

(SEQ ID NO: 312)
MSDEEVEQVEEQYEEEEEAQEEAAEVHEEVHEPEEVQEDTAEEDAEEEKP
RPKLTAPKIPEGEKVDFDDIQKKRQNKDLMELQALIDSHFEARKKEEEEL
VALKERIEKRRAERAEQQRIRAEKERERQNRLAEEKARREEEDAKRRAED
DLKKKKALSSMGANYSSYLAKADQKRGKKQTAREMKKKILAERRKPLNID
HLGEDKLRDKAKELWETLHQLEIDKFEFGEKLKRQKYDITTLRSRIDQAQ
KHSKKAGTPAKGKVGGRWK.

### Cardiac Troponin TI3 (TNNI3)

Exemplary therapeutic replacement genes nay comprise or consist of the amino acid sequence of Cardiac Troponin TI3.

(SEQ ID NO: 313)
MADGSSDAAREPRPAPAPIRRRSSNYRAYATEPHAKKKSKISASRKLQLK
TLLLQIAKQELEREAEERRGEKGRALSTRCQPLELAGLGFAELQDLCRQL
HARVDKVDEERYDIEAKVTKNITEIADLTQKIFDLRGKFKRPTLRRVRIS
ADAMMQALLGARAKESLDLRAHLKQVKKEDTEKENREVGDWRKNIDALSG
MEGRKKKFES

Pre-mRNA processing factor 31 (PRPF31)

Exemplary therapeutic replacement genes may comprise or consist of the amino acid sequence of pre-mRNA processing factor 31 (PRPF31) (autosomal dominant Retinitis Pigmentosa):

(SEQ ID NO: 487)
MSLADELLADLEEAAEEEEGGSYGEEEEEPAIEDVQEETQLDLSGDSVKT
IAKLWDSKMFAEIMMKIEEYISKQAKASEVMGPVEAAPEYRVIVDANNLT
VEIENELNIIHKFIRDKYSKRFPELESLVPNALDYIRTVKELGNSLDKCK
NNENLQQILTNATIMVVSVTASTTQGQQLSEEELERLEEACDMALELNAS
KHRIYEYVESRMSFIAPNLSIIIGASTAAKIMGVAGGLTNLSKMPACNIM
LLGAQRKTLSGFSSTSVLPHTGYIYHSDIVQSLPPDLRRKAARLVAAKCT
LAARVDSFHESTEGKVGYELKDEIERKFDKWQEPPPVKQVKPLPAPLDGQ
RKKRGGRRYRKMKERLGLTEIRKQANRMSFGEIEEDAYQEDLGFSLGHLG
KSGSGRVRQTQVNEATKARISKTLQRTLQKQSVVYGGKSTIRDRSSGTAS
SVAFTPLQGLEIVNPQAAEKKVAEANQKYFSSMAEELKVKGEKSGLMST.

### Progranulin (GRN) (FTD)

Exemplary therapeutic replacement genes may comprise or consist of the amino acid sequence of Progranulin (GRN) (frontotemporal dementia (FTD)):

(SEQ ID NO: 488)
MWTLVSWVALTAGLVAGTRCPDGQFCPVACCLDPGGASYSCCRPLLDKWP
TTLSRHLGGPCQVDAHCSAGHSCIFTVSGTSSCCPFPEAVACGDGHHCCP
RGFHCSADGRSCFQRSGNNSVGAIQCPDSQFECPDFSTCCVMVDGSWGCC
PMPQASCCEDRVHCCPHGAFCDLVHTRCITPTGTHPLAKKLPAQRTNRAV
ALSSSVMCPDARSRCPDGSTCCELPSGKYGCCPMPNATCCSDHLHCCPQD
TVCDLIQSKCLSKENATTDLLTKLPAHTVGDVKCDMEVSCPDGYTCCRLQ
SGAWGCCPFTQAVCCEDHIHCCPAGFTCDTQKGTCEQGPHQVPWMEKAPA
HLSLPDPQALKRDVPCDNVSSCPSSDTCCQLTSGEWGCCPIPEAVCCSDH
QHCCPQGYTCVAEGQCQRGSEIVAGLEKMPARRASLSHPRDIGCDQHTSC
PVGQTCCPSLGGSWACCQLPHAVCCEDRQHCCPAGYTCNVKARSCEKEVV
SAQPATFLARSPHVGKDVECGEGHFCHDNQTCCRDNRQGWACCPYRQGVC
CADRRHCCPAGFRCAARGTKCLRREAPRWDAPLRDPALRQLL.

gRNA Target Sequences

In some embodiments of the compositions of the disclosure, a target sequence of an RNA molecule comprises a pathogenic sequence. In some embodiments, the target RNA comprises a sequence motif corresponding to the spacer sequence of the guide RNA of the RNA-guided RNA-binding protein. In some embodiments, one or more spacer sequences are used to target one or more target sequences. In some embodiments, multiple spacers are used to target multiple target RNAs. Such target RNAs can be different target sites within the same RNA molecule or can be different target sites within different RNA molecules. Spacer sequences can also target non-coding RNA. In some embodiments, multiple promoters, e.g., pol III promoters) can be used to drive multiple spacers in a gRNA for targeting multiple target RNAs. In some embodiments, when the target RNA(s) or target sequence motif(s) is/are targeted and knocked down by the RNA-targeting compositions disclosed herein, then pathogenic or disease-causing gain-or-loss-of-function mutations are destroyed.

In some embodiments of the compositions and methods of the disclosure, the sequence motif of the target RNA is a signature of a disease or disorder.

A sequence motif of the disclosure may be isolated or derived from a sequence of foreign or exogenous sequence found in a genomic sequence, and therefore translated into an mRNA molecule of the disclosure or a sequence of foreign or exogenous sequence found in an RNA sequence of the disclosure.

A target sequence motif of the disclosure may comprise, consist of, be situated by, or be associated with a mutation in an endogenous sequence that causes a disease or disorder. The mutation may comprise or consist of a sequence substitution, inversion, deletion, insertion, transposition, or any combination thereof.

A target sequence motif of the disclosure may comprise or consist of a repeated sequence. In some embodiments, the repeated sequence may be associated with a microsatellite instability (MSI). MSI at one or more loci results from impaired DNA mismatch repair mechanisms of a cell of the disclosure. A hypervariable sequence of DNA may be transcribed into an mRNA of the disclosure comprising a target sequence comprising or consisting of the hypervariable sequence.

A target sequence motif of the disclosure may comprise or consist of a biomarker. The biomarker may indicate a risk of developing a disease or disorder. The biomarker may indicate a healthy gene (low or no determinable risk of developing a disease or disorder. The biomarker may indicate an edited gene. Exemplary biomarkers include, but are not limited to, single nucleotide polymorphisms (SNPs), sequence variations or mutations, epigenetic marks, splice acceptor sites, exogenous sequences, heterologous sequences, and any combination thereof.

A target sequence motif of the disclosure may comprise or consist of a secondary, tertiary or quaternary structure. The secondary, tertiary or quaternary structure may be endogenous or naturally occurring. The secondary, tertiary or quaternary structure may be induced or non-naturally occurring. The secondary, tertiary or quaternary structure may be encoded by an endogenous, exogenous, or heterologous sequence.

In some embodiments of the compositions and methods of the disclosure, a target sequence of an RNA molecule comprises or consists of between 2 and 100 nucleotides or nucleic acid bases, inclusive of the endpoints. In some embodiments, the target sequence of an RNA molecule comprises or consists of between 2 and 50 nucleotides or nucleic acid bases, inclusive of the endpoints. In some embodiments, the target sequence of an RNA molecule comprises or consists of between 2 and 20 nucleotides or nucleic acid bases, inclusive of the endpoints. In some embodiments, the target sequence of an RNA molecule comprises or consists of between 20-30 nucleotides or nucleic acid bases, inclusive of the endpoints. In some embodiments, the target sequence of an RNA molecule comprises or consists of about 26 nucleotides or nucleic acid bases, inclusive of the endpoints.

In some embodiments of the compositions and methods of the disclosure, a target sequence of an RNA molecule is continuous. In some embodiments, the target sequence of an RNA molecule is discontinuous. For example, the target sequence of an RNA molecule may comprise or consist of one or more nucleotides or nucleic acid bases that are not contiguous because one or more intermittent nucleotides are positioned in between the nucleotides of the target sequence.

In some embodiments of the compositions and methods of the disclosure, a target sequence of an RNA molecule is naturally occurring. In some embodiments, the target sequence of an RNA molecule is non-naturally occurring. Exemplary non-naturally occurring target sequences may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, a target sequence of an RNA molecule binds to a guide RNA of the disclosure. In some embodiments of the compositions and methods of the disclosure, one or more target sequences of an RNA molecule binds to one or more guide RNA spacer sequences of the disclosure.

In some embodiments of the compositions and methods of the disclosure, a target sequence of an RNA molecule binds to a first RNA binding protein of the disclosure.

In some embodiments of the compositions and methods of the disclosure, a target sequence of an RNA molecule binds to a second RNA binding protein of the disclosure.

Compositions of the disclosure may comprise a gRNA comprising a spacer sequence that specifically binds to a target sequence of an RNA molecule encoding Rhodospin protein comprising or consisting of about 20-30 nucleotides of the sequence of:

| (SEQ ID NO: 314) | |
|---|---|
| TGGGGTTTTT CCCATTCCCA GGACTGCCTC CTCCACCTCT AGCCCCAGGG GACTCTGTGC | 60 |
| TGCTTGGCTC TGCTCATTGC TCAATCCAGC CATCCCAGGG TCAGGGATCA GGTGGAAGCT | 120 |
| GGCAGTTTCA ATCTATCCTG TGGATAGAGT GTGAAAGCAA CAAAACCCAC CACCGTTAAT | 180 |
| ACCAACATAG GAGCTGAGCT TTTAATGGCC CAATTTGCCT TAGTCTCCAG GCAGAGCTGG | 240 |
| GTAAAGCTAG AGCTTCTGGC TTTGCTTATA TGGAGAAGGG GGAGCAGTTA CTGAGGCAGC | 300 |
| TTAATTCTGA CACCTCAGAG ATGTGGCCAG CTTTTTGGAG CAGATTCTCC AGAATGGAGA | 360 |
| ATGGACTAGC AACTGCTGAA GATGGGCTTG TCTGGCAAGG GAAACTGGAA ACTGGGGCCC | 420 |
| ATGAACATCC CCAAGGAAGG TAGGCCCAGT GGAATTTCCC ACTCTTTGTT TCTAAGCTCT | 480 |
| TCGAGATAAG GATGACATCA GGGACTCAGC TGTTAATTAA ATGTGGGCGG GTGAGCATGG | 540 |
| CTTCTAGAGG CTCCATGCTC TAGATGCTGG GACCCAGGTG CTAGAGCAAA AGAGCAGGTG | 600 |
| GCTTCCAGAG GCTGAGAGAA AGGCCTGTCT CTCCATAGGC CACATTGGGA AGGGGAGGCA | 660 |
| CGGGACCTGG GGCCCCACAC TAGGGGTGAG ACCCCAGGCC CAATCTCACC CTCATTGGGA | 720 |
| ACTTGGCCTT CACCGTCCCC CTCCCCCAGT GTTGTTTTTT CAGGTCTGAT GACTGCATTC | 780 |
| TGCATTCCTG TGACTGTCCC TGCCTACAGC CCAACCCCCA GCCCTGGTCT GGCCTTGATG | 840 |
| CCTAGCTAAT TTTTAAAAAC CTGCCCCAAG GTTGGGTGAA ACCCCATCAT CTGAATGCCC | 900 |
| AATCTCAAAA TGTTCACTAT CAGGAGGTGA TAATCATAGT AATTAACTAG TTACATTAAT | 960 |
| TGATGTTATT CACAACATTA ACTAGAATCT GTACAGCTTC TTGCTATTTA CAAAGTGCTG | 1020 |
| AAACACACAC ATAGACACAC ACACACCTCT TTTGGTCTTC TCAGTAGCTG CGTGTCGGCA | 1080 |
| GGACCAGGGA TCTGGGATTT CCATTTTATA GGAGAAGAAA GTGAGGCCCA GGGAGGGAAA | 1140 |
| AACAACTGCT CCATATCATT AGCCAAGTAT GAGTTGCTGC TGCTGCGAGG GTCTGAGAGG | 1200 |
| ATAGATATGT TCTCCCTTCC CATTCATTCC TCCATTCCTT CCTGCATCCA TCCAGCATTT | 1260 |
| ATTAAGCACC TACTGTGTGC CCCATTCTGT GCTAGACACT TATCCCTAAG CTGGGACACT | 1320 |
| TTTCCAGAAA GCAAGAATCC TCGTGTTCCT GAAAGATGAG TTGGGAGGAG GAGGGGCACA | 1380 |
| CATCCCGCTG GCCTTGGGGA ACGTGGGACT CCAGATCAGT AGGTCTTGGT GGATGTCCCT | 1440 |
| TCTCAGGCTG TCCCAGGTGA GTGAGGAGCC TCATTAATTA TTTCTTAAAA AAAAAAAAAA | 1500 |
| AATTAAGGAG CCTATGTGAC TTCGTTCATT CTGCACAGGC GCTGCTCCTG GTGGGATGGC | 1560 |
| TGTGGCTGGG GGAAGGTGTA GGGGATGGGA GACGCCTATA GTCGGCCACA GAGTCCTAGG | 1620 |
| CAGGTCTTAG GCCGGGGCCA CCTGGCTCGT CTCCGTCTTG GACACGGTAG CAGAGGCCTC | 1680 |
| ATCGTCACCC AGTGGGTTCT TGCCGCAGCA GATGGTGGTG AGCATGCAGT TCCGGAACTG | 1740 |
| CTTGTTCATC ATGATATAGA TGACAGGGTT GTAGATGGCG GCGCTCTTGG CAAAGAACGC | 1800 |
| TGGGATGGTC ATGAAGATGG GACCGAAGTT GGAGCCCTGG TGGGTGAAGA TGTAGAATGC | 1860 |
| CACGCTGGCG TAGGGCACCC AGCAGATCAG GAAAGCGATG ACCATGATGA TGACCATGCG | 1920 |
| GGTGACCTCC TTCTCTGCCT TCTGTGTGGT GGCTGACTCC TGCTGCTGGG CAGCGGCCTC | 1980 |
| CTTGACGGTG AAGACGAGCT GCCCATAGCA GAAAAAGATG ATAATCATGG GGATGGTGAA | 2040 |
| GTGGACCACG AACATGTAGA TGACAAAAGA CTCGTTGTTG ACCTCCGGCT TGAGCGTGTA | 2100 |
| GTAGTCGATT CCACACGAGC ACTGCAGGCC CTCGGGGATG TACCTGGACC AGCCGGCGAG | 2160 |
| TGGGGGTGCG GCGCAGGCCA GCGCCATGAC CCAGGTGAAG GCAACGCCCA TGATGGCATG | 2220 |
| GTTCTCCCCG AAGCGGAAGT TGCTCATGGG CTTACACACC ACCACGTACC GCTCGATGGC | 2280 |
| CAGGACCACC AAGGACCACA GGGCAATTTC ACCGCCCAGG GTGGCAAAGA AGCCCTCCAA | 2340 |
| ATTGCATCCT GTGGGCCCGA AGACGAAGTA TCCATGCAGA GAGGTGTAGA GGGTGCTGGT | 2400 |
| GAAGCCACCT AGGACCATGA AGAGGTCAGC CACGGCTAGG TTGAGCAGGA TGTAGTTGAG | 2460 |
| AGGCGTGCGC AGCTTCTTGT GCTGGACGGT GACGTAGAGC GTGAGGAAGT TGATGGGGAA | 2520 |
| GCCCAGCACG ATCAGCAGAA ACATGTAGGC GGCCAGCATG GAGAACTGCC ATGGCTCAGC | 2580 |
| CAGGTAGTAC TGTGGGTACT CGAAGGGGCT GCGTACCACA CCCGTCGCAT TGGAGAAGGG | 2640 |
| CACGTAGAAG TTAGGGCCTT CTGTGCCATT CATGGCTGTG GCCCTTGTGG CTGACCCGTG | 2700 |
| GCTGCTCCCA CCCAAGAATG CTGCGAAGGC CTGAGCTCAG CCACTCAGGG CTCCAGCTGG | 2760 |
| ATGACTCT | 2768. |

Exemplary gRNA spacer sequences of the disclosure that specifically bind to a target sequence of an RNA molecule encoding a Rhodopsin protein of the disclosure may comprise or consist of a nucleic acid having a sequence selected from any one of any one of SEQ ID NO: 619 to SEQ ID NO: 3361.

In some embodiments, exemplary gRNA spacer sequences and corresponding Rho target sequences comprises or consists of the sequences as detailed in table 1.

TABLE 1
Spacer sequences and target sequences
used for Rhodopsin targeting

| Spacer | Spacer Sequences | Target Sequences |
|---|---|---|
| Rho | ACATGTAGATGACAAA | CAACGAGTCTTTTGTC |
| guide 1 | AGACTCGTTG | ATCTACATGT |
| | (SEQ ID NO: 465) | (SEQ ID NO: 462) |
| Rho | TGAAGATGTAGAATGC | CGCCAGCGTGGCATTC |
| guide 2 | CACGCTGGCG | TACATCTTCA |
| | (SEQ ID NO: 409) | (SEQ ID NO: 463) |
| Rho | ACTGCTTGTTCATCAT | CATCTATATCATGATG |
| guide 3 | GATATAGATG | AACAAGCAGT |
| | (SEQ ID NO: 466) | (SEQ ID NO: 464) |

Compositions of the disclosure may comprise a gRNA comprising a spacer sequence that specifically binds to a target sequence of an RNA molecule encoding SOD1 protein comprising or consisting of about 20-30 nucleotides of the sequence of:

| (SEQ ID NO: 315) | |
|---|---|
| tttttttttt ttttttttag tttgaatttg gattctttta atagcctcat aataagtgcc | 60 |
| atacagggtt tttattcaca ggcttgaatg acaaagaaat tctgacaagt ttaataccca | 120 |
| tctgtgattt aagtctggca aaatacaggt cattgaaaca gacattttaa ctgagtttta | 180 |
| taaaactata caaatcttcc aagtgatcat aaatcagttt ctcactacag gtactttaaa | 240 |
| gcaactctga aaaagtcaca caattacact tttaagatta cagtgtttaa tgtttatcag | 300 |
| gatacatttc tacagctagc aggataacag atgagttaag gggcctcaga ctacatccaa | 360 |
| gggaatgttt attgggcgat cccaattaca ccacaagcca aacgacttcc agcgtttcct | 420 |
| gtctttgtac tttcttcatt tccacctttg cccaagtcat ctgctttttc atggaccacc | 480 |
| agtgtgcggc caatgatgca atggtctcct gagagtgaga tcacagaatc ttcaatagac | 540 |
| acatcggcca caccatcttt gtcagcagtc acattgccca agtctccaac atgcctctct | 600 |
| tcatcctttg gcccaccgtg ttttctggat agaggattaa agtgaggacc tgcactggta | 660 |
| cagcctgctg tattatctcc aaactcatga acatggaatc catgcaggcc ttcagtcagt | 720 |
| cctttaatgc ttccccacac cttcactggt ccattacttt ccttctgctc gaaattgatg | 780 |
| atgccctgca ctgggccgtc gcccttcagc acgcacacgg ccttcgtcgc cataactcgc | 840 |
| taggccacgc cgaggtcctg gttccgagga ctgcaacgga aaccccagac gctgcaggag | 900 |
| actacgacgc aaaccagcac cccgtctccg cgactacttt ataggccaga cctccgcgcc | 960 |
| tcgcccactc tggccccaaa c | 981. |

Exemplary gRNA spacer sequences of the disclosure that specifically bind to a target sequence of an RNA molecule encoding a SOD protein of the disclosure may comprise or consist of a nucleic acid having a sequence selected from any one of any one of SEQ ID NO: 3362 to SEQ ID NO: 4317.

Compositions of the disclosure may comprise a gRNA comprising a spacer sequence that specifically binds to a target sequence of an RNA molecule encoding PMP22 protein comprising or consisting of about 20-30 nucleotides of the sequence of:

| (SEQ ID NO: 316) | |
|---|---|
| TGAGTTACTC TGATGTTTAT TTTAATGCAT CTTAGTCCAC ACAGTTGGTA TAAAATCAGA | 60 |
| AAATGCAAAG CAAAAACAAA AGGTCTGGAG TCTTAGCATC AGAAGGGCAC CATATATACA | 120 |
| TCTACAGTTG GTGGCCAATA CAAGTCATTG CCAGACAGTC CTTGGAGGCA CAGAACAGCC | 180 |
| TAGACCCAGC CAAGCTCTAG GAACTCACGG TCCCAAGGAG TCTAGACGCT TGTTCTGATG | 240 |
| CTCCGACCGT AAGAAAAATG TGGGAGTGAT GAAGGCTTTA TGATTTACTC ATTATAGTAA | 300 |
| TAATAGCAGC CTAGCTAGGT ACAAAAGCAG TTATAAACCA TTTATATTAC ACAGAATTAT | 360 |
| TCAGGTCTCC ATTCTATCTT ATGTTGTAAA ATTGTTAATT GGATTTCCAG TGAGTTGTTT | 420 |
| AGATGATTAG TGATAATAAG GAATGGTAAA TCCATAGCAC CATTTCAAAG ACTTGTTGTC | 480 |
| ACTGATTTCT CATTTAGATG TGTGACGAAG ATACTCCACC TGTAAGGGCA AGTATGCCAA | 540 |
| TGCCACAAGC CGTGTTTTTG CAAGGGCTCC AGTTTGGGCA TTTTGTCCGT GTGCGCGTAA | 600 |
| AGCTTCACAC AGAGGTTCGG GCAGCGGCTG TTTCTGTTGG ATGCACTGGG TCACCCACCA | 660 |
| GAAAAGGGCT TTTGGACATT TGGGGTTTCT ACCCACACTT TGGTTTTCTA AATGAGGTGG | 720 |
| ACTGGGAGGG AGGTATCTTC TTTCAGATGA AAGGGAAGGG GCGAGATGGA GTTATCTTAT | 780 |
| TTCTGGGTAA AACAAAACAA ACAAACAAAA AACAAAACAA AAATACTGAG CTGGATTATA | 840 |
| CTGTTAGGAT GTAAAGTTCC TTAGCTACTT CTTTAAGGCT CAACACGAGG CTGATGGTCA | 900 |
| ACATAAAAAG CAAACAATAC TATGTACATA TATGTAAAAA GTGTTATAAA TAGGTTTTAT | 960 |
| AAACCGGAGA TATTATATAC ATCTTCAATC AACAGCAACC CCCACCTCCA CTGCTTTCTG | 1020 |
| TTTGGTTTGG TTTGAGTTTG GGATTTTGGG CTAGCTCTTT TTTCTTTGTC TGCTTTCTGT | 1080 |
| TTTCCCTTCC TCCCTTCCCT ATGTACGCTC AGAGCCTCAG ACAGACCGTC TGGGCGCCTC | 1140 |
| ATTCGCGTTT CCGCAAGATC ACATAGATGA CACCGCTGAG AAGGGCCAGG GGGAAGGCCA | 1200 |
| CCCAGGCCAG GATGTAGGCG AAACCGTAGG AGTAATCCGA GTTGAGATGC CACTCCGGGT | 1260 |
| GCCTCACCGT GTAGATGGCC GCAGCACTCA TCACGCACAG ACCAGCAAGA ATTTGGAAGA | 1320 |
| TTCCAGTGAT GTAAAACCTG CCCCCCTTGG TGAGGGTGAA GAGTTGGCAG AAGAACAGGA | 1380 |
| ACAGAGACAG AATGCTGAAG ATGATCGACA GGATCATGGT GGCCTGGACA GACTGCAGCC | 1440 |
| ATTCGTTTGG TGATGATGAG AAACAGTGGT GGACATTTCC TGAGGAAGAG GTGCTACAGT | 1500 |
| TCTGCCAGAG ATCAGTTGCG TGTCCATTGC CCACGATCCA TTGGCTGACG ATCGTGGAGA | 1560 |
| CGAACAGCAG CACCAGCACC GCGACGTGGA GGACGATGAT ACTCAGCAAC AGGAGGAGCA | 1620 |
| TTCTGGCGGC AAGTTCTGCT CAGCGGAGTT TCTGCCCGGC CAAACAGCGT AACCCCTTCT | 1680 |
| TCCAAGCAGA TTTCTTTGCA GCCAAATGCA AGGGATGTTA AGGCAAGACC CTCCCCACAG | 1740 |
| GGCAGTCAGA GACCCGCAGC CGACAGACTA AGCCTGCAGC TTCCAACCAG GCTCCCCGAG | 1800 |
| ATGTTCCCTG GTGGTGCTCC CTGTAACT | 1828. |

Exemplary gRNA spacer sequences of the disclosure that specifically bind to a target sequence of an RNA molecule encoding a PMP22 protein of the disclosure may comprise or consist of a nucleic acid having a sequence selected from any one of any one of SEQ ID NO: 4318 to SEQ ID NO: 6120.

Compositions of the disclosure may comprise a gRNA comprising a spacer sequence that specifically binds to a target sequence of an RNA molecule encoding PABPN1 protein comprising or consisting of about 20-30 nucleotides of the sequence of:

| (SEQ ID NO: 317) | |
|---|---|
| TTTTTTTTTT TTTTTTTTAC ACCCAAAAGG CCAAACAATT TTTATTTTCA AAAACAACTT | 60 |
| TATTCATGAC ACATATTAAA AAAAAATTCC CACCCCTGGA AATGAGCTAA AAAAATAAAC | 120 |
| AAAATCCACC TCCCACCTCC CTGTTCCCAC TTCCTCCCAT TCCCTCCAAA TAAAAGGGAA | 180 |
| AAAAGGCAAA GGAAAAAAAA AAAAAACAAA AAAACAAAAC AACTGAAAAA CAAAAACACC | 240 |
| CCTAAACCCC CCAAAACAAG GTAGTGCATT TCCCCAGGGG GAAGGGGAAT TTACACTGGA | 300 |
| GCCGCTGGGA GCGGAACGGA GATCTTCCGG CTACAGAAAC CTGCAAAGAA AGACACTCAA | 360 |
| AACAGAAAAA GAAACACAAA AGGAAACAAA ATAGATCACC AGGCAATCTG GAGGGGCAGG | 420 |
| GAGCCGGAGA AGAGGGGTGG GGTGGGTGGT AGACCTGGCT GGACAGGAGC AGGCAGGAGG | 480 |
| GGACTGTGAA AGGCGAGGAG AAGACGGAGG GAAGGTAACA AGCAGAACAG TTTGGTGTCC | 540 |
| TTCCAGAGCC CTGGGTAAAA AAAAAAACCT CCTACCACCC ACGCCCACCT ACCCTTGAGC | 600 |
| AGCCCCCAAG GGGGTGAAGT GGGGCAGGGA AACATGGGCA GCAGCTTGCG CAGTTGAGAC | 660 |
| GTGTCCATGG CGAATCCCCA GAGTGAATAA GCAGCCCCCT GCCCCACTCC CTGGGCCTTC | 720 |
| CCCTACTCCC CAAAGCAGGT CCCTCCTCAG CAGTTAGTTA TGGGATTCTC CCCCCTTCCA | 780 |
| CAGTATATCT TTTTTTTAAA AAATATTTTT TTTCCATCAA GGTCATCTTC TGTTTTTCTT | 840 |
| TTTTTTTTTT TTAATTCTTT TTTTTTTCCT TCTTTCCTCT TTTTTTCCTC TCTCTCCTCC | 900 |
| TAATACACAC TTTTTTTAGT AAGGGGAATA CCATGATGTC GCTCTAGCCC GGCCCCTGTA | 960 |
| GACGCGACCC CGGGGCCTGC TGTTAAAACC ACTGTAGAAT CGAGAGCGGG AGCTGTTGTA | 1020 |
| GTTGGTGGTC CGGGCGCGGT AGCGGGCTCG TGGAAAACCC CGGTCTGTTG TGCTGATGCC | 1080 |
| TGGTCTGTTG GTTCGTTTTG GGATCACCTT GATTTGCCTT CCTCTAAATA GGGACTCATC | 1140 |
| TAAGGCCAAG GAAGTCCTCA CTGACTCTTT GTCTGAGAAC TCTATATACG CAAACCCTTT | 1200 |
| GGGATGGCCA CTAAATTTGT CACACAGTAT GGTAACACGG TTGACTGAAC CACAGCCATG | 1260 |
| AAAGTGAGCT TCCAGCTCTT CTGCTGTTGC ACCATAGTCC ACATTGCCAA CATAGATGGA | 1320 |
| ACGGGCATCA GCCTCCATCT TCTCCTCAAT GGACATGATC ACCGGGCCAG CATTGCCTGG | 1380 |
| AGGTGGACTC ATATTCATCT GCTTCTCTAC CTCGTTCTGT AGCTCCTTTA GCTTCTCAGC | 1440 |
| TTCTTCCTCC ATCTCCCTGA CTCGAGCTTT GATAGCTTCC AGCTCCGGGT CCTCAATGGC | 1500 |
| GCCGTCCCCC GGGTCACCCT CGACCAGTCC CGGCTCCTCC TCCTCCTCTT GGCTGCCGGG | 1560 |
| GGCTCCCGAA CCAGGCCCAG GGCCCGGAGC TCCCGGGGGG GCGCGGGGCC GGGGCGGCTC | 1620 |
| CTCTTCGGGC TCGGGCTCCG GCTCGGGCTC CAGCAGCAGC TCCTCAGGCT CCAGTTCCTC | 1680 |
| AGACTCCAGG CCGTTCCCGT AGTCCCCTGC GCCCCCCGGG GCCCCCTCCC CGGCCTCCCC | 1740 |
| ACCGGCCCCG GGCACAAGAT GGCGCCGCCG CCCCGGCCCG GAGCCCCGAC CGCCCGCAGC | 1800 |
| CCCCGCTGCT GCTGCCGCCG CCGCCGCCGC CGCCATCGCC GCTCAGACTG GGGCCCGCCG | 1860 |
| CCCGGCGATT GGAGAGCTGC GCCGGCCACG CCGAGGACTC ATTAGTCAAG CTGCCTGCCC | 1920 |
| GTCACCATGA GCTAGTACTC CATTGGGGAA TATTACTTGG CAATCAAATA AGGCCCCACC | 1980 |
| TCTAAGGCGG GGCACTGCGC CAAATTCTCA AATCCCGGTA GGGGAAATCT GCCTGTCAAT | 2040 |
| CAACACGCGT CCCACCTCCT ATCGAGTCCT TAGGTAATAA TACCGCCACG CTGTGACGAT | 2100 |
| ATTCCTGCTT CTCCCCGGCC TACGGGCGGG CCCGCGAAGT ATGGGACGCT CCGTGATTGG | 2160 |
| CCCTAGCTAG GCGACTGGAA AGGACCAATC TTCCGATCGC CTCACCGCAG TGGCCCAGTC | 2220 |
| TCAGATGCCG ATTGGCTTGC GAGAGTCGAA GGGGTGACAC TCGTTTCGTG ACAGGTGAAC | 2280 |
| CTTGCCCCCG AAAGGACTGC CGGGCTTCAA ACTTGGGAAA CCCGAGGTCA CATGACTAGC | 2340 |
| CAGTCCTAGG GGGCCGCCAT CTTGATACTA CTGCTTGCCA GCTAGTGAGC TGTTGGCCGG | 2400 |
| GTGAGGCCCA AAACAGAGCA GCAGTTTCAG GAAACTTGTA TCTCGACCAG GAAGCACCAG | 2460 |
| TAGATGGGAT GTTGCTGAAA ATGGAGGTTG TGAATGAAGC ATTCCAGGAG GGAGCTTACT | 2520 |
| TTCCCCATCC AGGTTATTGG CACCATCATC CACTAGCTCT CCCGCACCAG AAAGCAGGGA | 2580 |
| GGATTCCTCA GTCCAGAGCT ACTAGTCACA AGTCCTGTCT GTCCCGCCCT CTTGCGTAGG | 2640 |
| CCTTCTGCTC CCCAGTTCCA TTTTCTTTTT CCTGGACAGC TTCCAATGTC ACCCCTCCAA | 2700 |
| TCTGCACCGC TAACAGACTG GCCCCCCTTT GCTGGCGAGG TAAAGTCTCA AAACCGTAAA | 2760 |
| TCACGGCCTT CGATACGCCA GCATGTGGTT ACTTTGTGGA TGTTGTTTCC TTCCACTCTT | 2820 |
| CTCGTTCCTT TGGGTGTACC TGCACCCAGT CTGTGCCTCT AACATGTAGT CCCCCTTCAA | 2880 |
| TCAAACCACT GCAAACCCCA GCTTCCCCTC ATTTCCCAGG ACAAGTGGGC CTATCTCCAC | 2940 |
| GGCGCGCTTA ATTGTTTTAC TGTTTCCTAA CTAGGTTGTG AGCGCCTGCA GATGAGGGGC | 3000 |
| CGGTTCCTAT TTATATTCGC ATCTCCACGG CCTGGCAATA TGCCTACCAC ATAATGTCCT | 3060 |
| GTTAGATGTT TGTTGATTGA ACAGGCATTG ATTGGGGATT TGGGTGCCAC CCTTCATT | 3118. |

Exemplary gRNA spacer sequences of the disclosure that specifically bind to a target sequence of an RNA molecule encoding a PABPN1 protein of the disclosure may comprise or consist of a nucleic acid having a sequence selected from any one of any one of SEQ ID NO: 6121 to SEQ ID NO: 9213.

Compositions of the disclosure may comprise a gRNA comprising a spacer sequence that specifically binds to a target sequence of an RNA molecule encoding KCNQ4 protein comprising or consisting of about 20-30 nucleotides of the sequence of:

| (SEQ ID NO: 318) | |
|---|---|
| tcctcttgaa aactttaatg aaaccaataa gttaataagt taacaaagtg aggtacttgc | 60 |
| atatccgaga accgagttga tggcggaacc tcgatcgtgg aaggaaggag ggcttcttcc | 120 |
| catgtctcct tggtcatagg gtaatcccat tctcctggcc ctctccacag ctgtctctga | 180 |
| gtgggtggag gggtggctct ctggggacag tgcagtgcag gtggggtgag ccaggagtgg | 240 |
| ctccatccag ggcatgacgg tcacatgcag agggcatgtt ggggcagagg ctgggcacca | 300 |
| tggactgagt aagatccagt ccaagccctg gtgctggcgc atgcaggtcc ccagcacgag | 360 |
| atgcaggcag cgtggcctcg gccccttgtt cccacagagg gttgccattg gtccactacc | 420 |
| cctcaccttc ctgccacctg cattggcttt gcagaagagg agcaaagggg tggggataag | 480 |
| aaagtgttcc tggagtgaat gggggctgac gcctggatgt ggcgctgcca attgaagtga | 540 |
| agaagtgggg tgtggaggga ggggagacag tgcatgtgaa agccggatgc gcggttggta | 600 |
| tagctattac atgcattctt attctctctg ggagttagag tgactaggaa ggcacaggcc | 660 |
| tggctagaag tgggcctggg gtctctccag tggtgtcagg cctgggcccg agttgtgctg | 720 |
| tggagacacg gaggcgggcc cttggggaca ggcagaggca gtgggttagg gctcctggac | 780 |
| cagagaggag gagatcttct tgaagaaggg gagcgggtaa aggtctgggt ggggcctgga | 840 |
| gagctctcgg gttacttcaa agctccttct cctgctcaga tggagttggg ggtcttcctc | 900 |
| ttagtggtcg tcaccctgca aggccagtcc actggcatct gctgcagagg cccaggagcg | 960 |
| gctgagtggc tctgcactca tgtctccaag tgtgtcacgt cattgcatgt ttaagtgtga | 1020 |
| gaggctttct tcctgtctgg agaaactaaa aggctatgtt tgtgcgatcg tccacgaagc | 1080 |
| actgtctggg agttttgggg aggatacaga aacctgtgct gactatgagt ggtgtctgga | 1140 |
| gggccaagga ggggagtggc cacccagcag gaggagggag cccctgggag ctgacacttg | 1200 |
| caaatgggaa gaaaggccca ggcattgccc ttaggagagg gcaggggaga aacaggcccc | 1260 |
| atgctgggag gggaaggagt gataagccaa agaataggga cagggtggtg gccaagcagc | 1320 |
| ctgagaccag ctactgctgg gggcccagct cccaatgggg gagggacaga gagaccccat | 1380 |
| gggggtcccc aaagttgcat atttaacatg gtttgcatat atggtgtcca cctgccttgt | 1440 |
| ggcagggtcc tgtgctttgt gggtacctgt gggggcacct tggcccaccc ccaagtgggg | 1500 |
| cggggacttg gcgggagggc ttccctgctg tgtccgaagg gtgggccaga gacctcacct | 1560 |
| tgcatagctg gaagctgggt cagggcccag ggccccagcg cccgctccca ctcccggggc | 1620 |
| tgtgcccctg ccaggtgggc cacatagggc cttgatggag tgggcagggc actggggggc | 1680 |
| ccagaggccg cgggacggcc gctgctgctg ccctctgtgc gacctggcat ctcacgctcc | 1740 |
| tgaggtgggg aaggggcgct ggcacccaca gcaggtacca cgcccactca ggcagctcaa | 1800 |
| tactgcgtgt ggtctctctc cccgtgaggg agtgagttca agtacgagag gagtccggag | 1860 |
| gcctcagagg gcagtcggag cgccaggccg cggggctggc cgtgtgctgc cctgcctctg | 1920 |
| agaagtccct cagtccatgt tggtgctgac cgagcgggag atgctgagcg tctgtgcgga | 1980 |
| gacggagatg tcctcgtggt ccacagggct gtggtagtcg gaggtgatgt cggggtcgaa | 2040 |
| cagcggcact tgcacggcgc ccaggctggc cgaggtgcca gagcgcaggc agcgcgaata | 2100 |
| gaagcccaac agcaggtcca gcttgtgctc gatggactgc acctgcttct ccaccttgac | 2160 |
| cacgcgtccc atcatgctga tttcatccac cacctccgcg tcggagggcc ccttgtcgcc | 2220 |
| cttctcccgg gccttcctgt ccccgggccc ccgacccaca atttggtcca cccgagtttg | 2280 |
| caggctcttg atccggccca gcatgtccag gtggcctgct gagtactgct caatgacgtc | 2340 |
| cttcacgtcg tacggtcgca gtgtctcctt gaatttcctt ttggccacca ggaacttgag | 2400 |
| aatcctgatg gagcggatga ctgtcttcac agcaggcatg atgtcgtcca ccgtgagctc | 2460 |
| acactggtag ctcttctcct ctgctacttc ctctgagggg gcatcctcag cagaggtgcg | 2520 |
| gggtttgagt ctcagagatg cccggaagcg ggtgcggtca ttgaagctcc agctcttttg | 2580 |
| caccttggtg gggctggtgg cctcacccac ctgctcgctg cttggggagg tgggcattgt | 2640 |
| tggaggtgcc agatgctgct tggaaggacc cgtccgccgc tgggagctgc ccatgcggat | 2700 |
| gcggtctttg atgcccatcc ggctgctttc cccagggcag aaggaggtgc tgcccggccg | 2760 |
| gtggcaggtg gcaacgggcg ggtaacggga gggtgctccg tcgggtaccg gcgcccgccg | 2820 |
| cacctccagg ggccgtaggc ccccattgcg ggcccgttgc acgtgctcaa acaagagggc | 2880 |
| cagctctctg aaggatggga ggatactgtc atagtagtac caggtggctg tcaggtaggc | 2940 |
| ccggctcata tcggtggagt acaggcgcca ggcagcctgg atgaggttgg ctgccggcat | 3000 |
| cctccgcttc tcgaagtgct tctgccggtg ctgctcctgg accttcaggg caaagccgga | 3060 |
| gcctaggatg ccggcaggca gggcaaagaa agagatgccc agtaaggcga agccagcagc | 3120 |
| caggaccctg cccagccatg tgtgcggtgt cttgtcacca tagccgatgg ttgtcaatgt | 3180 |
| aatcgtcccc caccagagcg agtcggcgta ggaggagaag tcggagttgg cgtccttctc | 3240 |
| agccaggtag accaggaagg aggcgaagat gagcaccagg aacccgatgt accaggcggt | 3300 |
| gatcagctcc ttgctatgcg cgtagaccac tgagcccagc agcttccagg tgccgccgcg | 3360 |
| gcggtccatg cgcaccatgc gcaggatctg caggaagcgc atgctgcgca gcgcggacgt | 3420 |
| ggcgaagatg ttgccctggg tacccgcggc gatgacggcc accgaggcca cgaacacgat | 3480 |
| gaagtcgatg acacagaagg gctttctggc aaagcggaag cgaccctgcc atcctcggta | 3540 |
| gcggcagcag catccggcgg accagacccg gacgatgtac tccaagccga aaaccacgat | 3600 |
| catcacgaat tccaagatga ggagacactc gttggcaagt tcctggtgct cctggatagt | 3660 |
| ggacagcaca gacagcacca ggcagctgaa gaccagcaaa aatatgaaga cgtggtagac | 3720 |
| gaaggcccag ccgcggggcc gctccagcac gttgtagacc cagttctgca ggcggcggta | 3780 |
| gcgcttgtgc gcggccgagg agcgctggcc gcaggcggag cccgagccgg agcccggccc | 3840 |
| agggaggggc gcgcccggcg gcagggggct gcccaggagg ccgaggcggc gcggggagcc | 3900 |
| gcccccgccc gcctcgccct gttcgctctg cacggccgtg agcgccacta gctccgcgcg | 3960 |
| gggggcgtcc ccgggcgggg gacccaggcc gaggcggcgc gggggggcct cggccatggg | 4020 |
| cggcgccggg ctgggggcgc cggggcccgg gcacggtccg gggcgggggc gcgctcgggg | 4080 |
| cgctcagaga cgcatggctc ggacccgggg ccagaggggc gacccggggc gggcgcgggc | 4140 |
| ggcgggggct aggggccggg ccggggccgc gggcgggcgc tcggagcctg ggggccgccg | 4200 |
| gagcccgcac tgacctcccg cttccccggc gactggggct gctctttccg actccaactc | 4260 |
| tcttattacg cgctccatgc cgctcgcctt tccacctgcc accggcgcgc gccgctcaca | 4320 |
| tgtc | 4324 |
| | 4324. |

Exemplary gRNA spacer sequences of the disclosure that specifically bind to a target sequence of an RNA molecule encoding a KCNQ4 protein of the disclosure may comprise or consist of a nucleic acid having a sequence selected from any one of any one of SEQ ID NO: 9214 to SEQ ID NO: 13512.

Compositions of the disclosure may comprise a gRNA comprising a spacer sequence that specifically binds to a target sequence of an RNA molecule encoding CLRN1 protein comprising or consisting of about 20-30 nucleotides of the sequence of:

| (SEQ ID NO: 319) | |
|---|---|
| agcatctgga aactcggtgt gttctgatgt ctgctggcga atagcgaatt gacaccagag | 60 |
| caagttattt ctcaggtata cggttgtttc atccttgtaa atagttccaa agggaaacag | 120 |
| tgttttattt taaggagtac tttcaaacct attatatgag ggctgctgag tactcagcac | 180 |
| ctgtggtcag aggcctagtg atctgtttgc tgtcattctc tgctttttcc ttggtgcttt | 240 |
| ctggaggaca gcaggttgag gatgaaggaa gggtcagttc caggctcagc tgtggccttt | 300 |
| agtcagctgc agatcaattt gatgggtaat tcaggggaaa aaaaaagttg acctgggtca | 360 |
| tgcttggtga cagccagaac aagaccaaga tgatacagtg ataccgtcat aatcccagat | 420 |
| ttaatataat tttcataatt gcatattagt actcgagaca ctatagctag aaaaacagcc | 480 |
| cctaataagt cattttgcat caaatgtact aagcagagat catttttcat gattcctcag | 540 |
| tggtcctaac aattatgttc attgaaagta ctgtcgtgaa tgtaattggg actcaggcac | 600 |
| gggaggaaaa ataccctaag cttggttttt tcttcttttc ttcttttaga gtttgcagat | 660 |
| tttgaccaac agacatggtt aataagacta tgctttttta aagcctatat tttatattta | 720 |
| ttttattttt taattttgtt agtgacaggg tctcacttta ttgcccaggc tgtaactcga | 780 |
| actcctgaac tcaaatgatc ttcccacctt ggcctcctga agtgctggaa ttacaggtgt | 840 |
| gagtcaccac gcctggccta agagtatact ttaaacaaat tttttaaaat gtgtgttgat | 900 |
| acattttata gatgttcatt taatacacta ctgttttagg aaagcgattg cagctcagtt | 960 |
| ttctgaaatc tggcaacaaa tgtgtggata tattagagat attatttgtt tttattaaaa | 1020 |
| tatattccat gtgcctttga tatctttttg ataggaagac atcttacaca cacacacaca | 1080 |
| cacacacaca cacatatata tatatggagt aacaatttgt cgattctagt caactgcctt | 1140 |
| tgactacctg ggtcaagcaa tttcccacca gataaaacaa cttttcaaag ccttccttct | 1200 |
| gcttccctta ctttccagcc tgtatcctta gtacgtaatt tgtaaacatt gtcacgaagg | 1260 |
| gtcctgatgc tttaatatat gcagactaaa aggatatgca aaattaacca catctaaaag | 1320 |
| tgaccaaagc aagtctactc ccttgtaaaa ttatagaaag gtttgccttt cagtacatta | 1380 |
| gatctgcagc tacattagtt gtttctgcgt ctttagattt tgcaaaaggg aactgaaatc | 1440 |
| cagcaagtcg tattaggagc ccattcagaa aatgaacaaa aaagcaaaag aaaatgaccc | 1500 |
| agaatgaggt ggtatatttt tcactttgcg ttttgtagac ataagtccct tctttataat | 1560 |
| ttgcaatttt ttctgagagg tgatggattt tcacttcaga ggcaaacaat atcatgacaa | 1620 |
| gacagccaca ggagcctgaa atgaagctca aaaggtacag ccctagggga ccatgcagag | 1680 |
| tttcaaaagg ttttccaaaa gcattgtaca tgaagaaggc tgtccccacc atggttaaca | 1740 |
| caataaggat ggcagagaag agaatgacat tgacgtggat gctcactggg attgctttga | 1800 |
| gcaaatctgg aaaaaatgag aaccgaaagg gccttgctcc caacccacac tgcctcacac | 1860 |
| cctctccgtg gaaaagcccg tactgcattt cacccataaa cttgtccagc tcctgccctg | 1920 |
| aggcattgac gagcagagct cccgttttgc agaggacagt ggctttgatc cacaacggtg | 1980 |
| tccccaaggc tgtcacaact ccgagggcac atgcaaaact gaacactccg gccatgcaaa | 2040 |
| aaatgatttt cttctgttgg cttggcatga tgagaaacgg cttctgt | 2087. |

Exemplary gRNA spacer sequences of the disclosure that specifically bind to a target sequence of an RNA molecule encoding a CLRN1 protein of the disclosure may comprise or consist of a nucleic acid having a sequence selected from any one of any one of SEQ ID NO: 13513 to SEQ ID NO: 15574.

Compositions of the disclosure may comprise a gRNA comprising a spacer sequence that specifically binds to a target sequence of an RNA molecule encoding APOE2 protein comprising or consisting of about 20-30 nucleotides of the sequence of:

| SEQ ID NO: 320) | |
|---|---|
| tgcgtgaaac ttggtgaatc tttattaaac tagggtccac cccaggagga cggctggggc | 60 |
| ggggacaggg tctcccgctg caggctgcgc ggaggcagga ggcacggggt ggcgtggggt | 120 |
| cgcatggctg caggcttcgg cgttcagtga ttgtcgctgg gcacaggggc ggcgctggtg | 180 |
| cccacggcag cctgcacctt ctccaccagc ccggcccact ggcgctgcat gtcttccacc | 240 |
| aggggctcga accagctctt gaggcgggcc tggaaggcct cggcctgcag gcgtatctgc | 300 |
| tgggcctgct cctccagctt ggcgcgcacc tccgccacct gctccttcac ctcgtccagg | 360 |
| cggtcgcggg tccggctgcc catctcctcc atccgcgcgc gcagccgctc gccccaggcc | 420 |
| tgggcccgct cctgtagcgg ctggccggcc agggagccca cagtggcggc ccgcacgcgg | 480 |
| ccctgttcca ccaggggccc caggcgctcg cggatggcgc tgaggccgcg ctcggcgccc | 540 |
| tcgcgggccc cggcctggta cactgccagg cgcttctgca ggtcatcggc atcgcggagg | 600 |
| agccgcttac gcagcttgcg caggtgggag gcgaggcgca cccgcagctc ctcggtgctc | 660 |
| tggccgagca tggcctgcac ctcgccgcgg tactgcacca ggcggccgca cacgtcctcc | 720 |
| atgtccgcgc ccagccgggc ctgcgccgcc tgcagctcct tggacagccg tgcccgcgtc | 780 |
| tcctccgcca ccggggtcag ttgttcctcc agttccgatt tgtaggcctt caactccttc | 840 |
| atggtctcgt ccatcagcgc cctcagttcc tgggtgacct gggagctgag cagctcctcc | 900 |
| tgcacctgct cagacagtgt ctgcacccag cgcaggtaat cccaaaagcg acccagtgcc | 960 |
| agttcccagc gctggccgct ctgccactcg gtctgctggc gcagctcggg ctccggctct | 1020 |
| gtctccaccg cttgctccac cttggcctgg catcctgcca ggaatgtgac cagcaacgca | 1080 |
| gcccacagaa ccttcatctt cctgcctgtg attggccagt ctggaggcca ggggttccca | 1140 |
| gggtcccagc tctttctaga ggcccctgag ctcatccccg tgcccccgac tgcgcttctc | 1200 |
| accggctcct ggggaaggac gtccttcacc tccgctgggg ctgagtag | 1248. |

Exemplary gRNA spacer sequences of the disclosure that specifically bind to a target sequence of an RNA molecule encoding a APOE2 protein of the disclosure may comprise or consist of a nucleic acid having a sequence selected from any one of any one of SEQ ID NO: 15575 to SEQ ID NO: 16797.

Compositions of the disclosure may comprise a gRNA comprising a spacer sequence that specifically binds to a target sequence of an RNA molecule encoding TNNI3 protein comprising or consisting of about 20-30 nucleotides of the sequence of:

| (SEQ ID NO: 321) | |
|---|---|
| tttcagctca gagagaagct ttattcctca gggccctcct cagggcaggg gcagtaggca | 60 |
| ggaaggctca gctctcaaac tttttcttgc ggccctccat tccactcagt gcatcgatgt | 120 |
| tcttgcgcca gtctcccacc tcccggtttt ccttctcggt gtcctccttc ttcacctgct | 180 |
| tgaggtgggc ccgcaggtcc agggactcct tagcccgggc ccccagcagc gcctgcatca | 240 |
| tggcatctgc agagatcctc actctccgca gggtgggccg cttaaacttg cctcgaaggt | 300 |
| caaagatctt ctgagtcaga tctgcaatct ccgtgatgtt cttggtgact tttgcctcta | 360 |
| tgtcgtatct ctcttcatcc accttgtcca cacgggcgtg gagctgtcgg cacaagtcct | 420 |
| gcagctccgc gaagcccagc ccggccaact ccagcggctg gcagcgggtg ctcagagcgc | 480 |
| gccccttctc tccgcgccgc tcctccgcct ctcgctccag ctcttgcttt gcaatctgca | 540 |
| gcagcagagt cttcagctgc aattttctcg aggcggagat cttagatttt ttcttggcgt | 600 |
| gcggctccgt ggcataagcg cggtagttgg aggagcggcg tctgattggg gctggtgcag | 660 |
| ggcgaggttc cctagccgca tcgctgctcc catccgccat gctgagactc aggccgggaa | 720 |
| tggcaggagg cagggcgagg acaggggcgt ttggagggtc agtgaggggg ccgcccgggt | 780 |
| gaccttcagg gtcccaggga ccgtcagtct cctccgggct gcttgagact ccccgaggac | 840 |
| act | 843. |

Exemplary gRNA spacer sequences of the disclosure that specifically bind to a target sequence of an RNA molecule encoding a TNNI3 protein of the disclosure may comprise or consist of a nucleic acid having a sequence selected from any one of any one of SEQ ID NO: 16798 to SEQ ID NO: 17615.

Compositions of the disclosure may comprise a gRNA comprising a spacer sequence that specifically binds to a target sequence of an RNA molecule encoding BEST1 protein comprising or consisting of about 20-30 nucleotides of the sequence of:

| (SEQ ID NO: 322) | |
|---|---|
| aacgagtatt tgtatttatt aaactcatta gtttgggcag tatactaagg tgtggctgtc | 60 |
| ttggattcag atagaactaa gggttcccga ctctgaatcc agagtctgag ttaaatgttt | 120 |
| ccaatggttc agtctagctt tcacagtttt tatgaataaa aggcattaaa ggctgaagta | 180 |
| gtctgggatt tttatctatt aagctaacca tttgattcag gctgttgtag gacatgttct | 240 |
| tcagtgtgga cagctgtatg gctgtgactg gatcagtgtc ctgctggtgt acacacaggt | 300 |
| gaggacctgg ctggcgaagc atccccatta ggaagcaggt taggaatgtg cttcatccct | 360 |
| gttttccaag gcccaataag gatccatgtg atctttgagt gtagtgtgta tgttggttgg | 420 |
| tgattgttcc aaaggttctt tgaggtgatt ttcggggatc tctggcatat ccgtcaggtt | 480 |
| aaactccaca gttttcctcc tcacttgaga tacttctggg tgctccatca aggccccatc | 540 |
| gctctctgag agcaattcaa aacttttctt ggccccagaa ctcacagtct ttaagctttt | 600 |
| gtctttggtg tctatgcctg tgacactgtg aagctttgac ggcgctgatg gttctagggg | 660 |
| gaagaacatg ggagtggggc tgaggggcgt ctgtggggca ctgtagtagc ctggcctctg | 720 |
| atacagtggg gcagacttga aggcgtccac agccttaagc ttccaggcct tgttgtcttc | 780 |
| ctggccccta acgttctgtt tggctgcctt gtggtttttg ggcaggccct cgtggagaag | 840 |
| ggattccctc ttgggccaca gtagtttggt ccttgagttt gccctgggag gatggtgatc | 900 |
| atgggactgc aggcctagga agcggccaat gatgccagcg tgagcatcct cctcgtcctc | 960 |
| ctgattgggc tggaactcca tctcctcttt gttcaggctg atgttgaagg tggagcccat | 1020 |
| aaaggaggct cgacggaact gggcggaagc agctgtgtag gggggctgtg gctcgggctt | 1080 |
| attccagtac atgtccggct ccatccgagg caggtcctgg tgcatctcat ccacagccaa | 1140 |
| cagggacacc tgcaaattcc tgtcgacaat ccagttggtc tcaaaatcat catcatcctc | 1200 |
| tccaaagggg ttgatgagct gctctgccac cttcagccag ccaacataga agaagaactg | 1260 |
| caggaacgtg aagacgggca caacgaggtc cagctcatgg ccagggtagg ccttggctgg | 1320 |
| gttcagaaac tgccgcccaa ctagacaagt caggaagaag ctgtacaccg ccacagtcac | 1380 |
| cacctgtgta tacaccagtg ggatactaat ccagtcgtag gcatacaggt gtccacactg | 1440 |
| agtacgcaag gtgttcatct cgttcagcag gctctggagc aggatagggt cccggattcg | 1500 |
| acctccaagc cacgccttca ttgacaggtt ggcaaaccac acccagggca cccagaacat | 1560 |
| gttgtgtggt aggctcagtt tctccaactg cttgtgttct gccggagtca taaagcctgc | 1620 |
| ttgcaccagg tgctgggcgc tggggaagcg cttgtagact gcggtgctga cgctgcgcag | 1680 |
| gatgagcacg ttgcccaggt tggcgtagcg gatgagcgtg cgccgcagca gccggccttg | 1740 |
| ctcgtccttg ccttcgacga agcccgacac caggctcatg aggcggtcgg gccacggcag | 1800 |
| gttctcgtac tggttccacc agcgggtcac gaccagcgtc acgtagaagc ccagcacgaa | 1860 |
| ggaaatgggg atgagctgga tgtagctgtc gcaatacaga gtcagtttct caaacatcag | 1920 |
| ctgttgttct tccgtgaggg ccagcctata aataaagcgg atgatgtagt agcagagcag | 1980 |
| gaagattaag aactcgccat atagcagctt gtagatgctg ccccgccagc acagcagcag | 2040 |
| gcgggagaag gagcctaagc gggcattagc cacttggctt gtgtaagtga tggtcatggc | 2100 |
| caggcagtgg gctgcagcag gtgggcttgg gtcaggtggg gttccaggtg ggtccgatga | 2160 |
| tcccacagaa ggtctggcga ctaggctggt gggactccct gggactctgt | 2210. |

Exemplary gRNA spacer sequences of the disclosure that specifically bind to a target sequence of an RNA molecule encoding a BEST1 protein of the disclosure may comprise or consist of a nucleic acid having a sequence selected from any one of any one of SEQ ID NO: 17616 to SEQ ID NO: 19800.

Compositions of the disclosure may comprise a gRNA comprising a spacer sequence that specifically binds to a target sequence of an RNA molecule encoding MYBPC3 protein comprising or consisting of about 20-30 nucleotides of the sequence of:

| (SEQ ID NO: 323) | |
|---|---|
| tgagttctct gtgactgcac ttatctttta ttgcccaata aacattggga agacatagca | 60 |
| ggccagaaag gcctgtcccc agacattgtt tcttgaggcc accctccttt taccccaaag | 120 |
| atccaggggc ttccttcagg agccctgtgg accagtctgt gcaacaccca ctcaggactg | 180 |
| cccgacaact gccctgctga tcccccatcg cagcacagga gacacacttg tcacacatac | 240 |
| atccaacagt agggaggggt ttccccaact tccctccagg ctcctggcac ggggctggca | 300 |
| tccggttgta cctggccatc cccaggagcc agcctggtca ctgaggcact cgcacctcca | 360 |
| ggcggcactc acaccgtgcc tcgccctgta agttggtggc cctgcagaca tagatgcccc | 420 |
| cgtcaaaggg gcagggcttt ctaatctcca gagtcaacac tccctgcttg ctgaacatgc | 480 |
| ggaagcgggc gtcttctccc aggtccaggc cattcttgaa ccaggaaatc ttgggcttgg | 540 |
| ggctaccccg gacagcacag cagagcatag cagtgtagcc cgcgatgacc gagcggttca | 600 |
| ccaggggctg ggtgaagctt ggggcctcgg agaagtccag ggccttatag ttgggtggct | 660 |
| cataggtgat gcctggtctg gggataaaga cgggctcctt ggtggtggcc gctctgtcac | 720 |
| taaagccaac catattctgg ctgaagacgc ggaagtagta gccattgcca atgatgagct | 780 |
| ctggcaccac gcagtgggtg cggcggtaat gctccaagac ggtgaaccac tccatggtct | 840 |
| tcttgtcggc tttctgcact gtgtaccccc agagctccgt gttgccgaca tcctggggtg | 900 |
| gcttccactc cagagccaca ttaagacccc aggcgtcagt cacccggaga tcctggggag | 960 |
| gacttggctt gtcaacaacc tgcagcacca gcgtggcctt gtcctccatg ttctcaatgc | 1020 |
| gcaccgtcac ctggtaagtg cctgaatgca cgcggcgagc ggcccggatg aacaggatgg | 1080 |
| tgtctgtggg gctgttgcgg atgctcacct cctcgcctgc caggggctgc ccctctttgg | 1140 |
| tccaggtcac ctgaggccgg ggcttgccct ggaaagggat gagaaggttc acaggctccc | 1200 |
| cgaccttctt ctgaatggtc tggcgcaggt gcctgggcag ctgaagccgt ggccgttgca | 1260 |
| ggatctcctg cactgtcacc ggctccgtgg tggtaacagg ggctccaggc cctgccatat | 1320 |
| tgtgtgcccg cactcggaaa agcagccggg cccccgtggg caggtccttc accagtatcg | 1380 |
| atgtgtgctc tgtcagcccc tgcagggcag ccacccactc tgagcagccc tctgggcagt | 1440 |
| actccacgct gtagccatcc aggcctcctg ctcccacgcg ctctgggggc cgccacttga | 1500 |
| gggagaccgt ggtgtcagag acgtcctcta ctgccaggtg ggtgggttcg ctggggggac | 1560 |
| cgataggcat gaagggctgg gaggcagggc tgggcctgga catgccgatg gcgttgaccg | 1620 |
| cgtagacgcg catctcgtac accacgccct cgatcatgcg ccgcgcttca tgactcagct | 1680 |
| cctgaatcag gtcgaagttc agccgcatcc accggtagct cttcttcttc ttgcgctcca | 1740 |
| ggatgtagcc caggatgggc tgcccgccat cgtaggcagg cggctcccac tgtactgtgc | 1800 |
| aggagtcctc tcccacgttg ctgatcttgg gggccgcagg tgcgtctggc acgtcgatga | 1860 |
| ccttgactgt gaggttgacc tggtcctcgc ccacagggtt cttcactgtg accgtgtaga | 1920 |
| cgccctcatc ttccttctct gccccctcga ccgtgaagat gctgcggtcc ttggtggtct | 1980 |
| ccacgcggac ccggccctcg gtctcacaca gcagcttctt gtcaaacacc cactcatcgc | 2040 |
| tgtcacctgt gtcctctggg gcatctgggg ctggcctggc tggggcctta ttcccctgcg | 2100 |
| tgatagcctt ctgccagatc acagtgggag cagggtcccc agagataggg acgtccagac | 2160 |
| gtagcttatt tccagctaca accacaatgg tgtctggtat gcggcctggg cagtccaggt | 2220 |
| ggatcttggg aggttcctgc ctgggtacga agtcaatctt gacctccatg aagtggagct | 2280 |
| tggctgacag gttgcaggcg aagccctcgg gcacaaagct gtagtcagcc tcgtcggcag | 2340 |
| gtgtgacgtc gtcaatggtc agtttgtgga cccgcccgat gtgggacacc tttatgcggc | 2400 |
| tgtcgggcac cagctccttc ccattcttca gccacacacc ccgaacattc tcatctgaga | 2460 |
| cctcacattt gaacaccgcc tggtcctttg cgcccaccat caggtctgcg atgctctggt | 2520 |
| acacctccag cttcttttcc tgcacaatga gctcagccag cgcctggccc ccgctagtgc | 2580 |
| acagtgcata gtgccccgcg tcctccagca tggcctcgtt gatgatcagg tggtgtctct | 2640 |
| gcccgtcctt cttgaaccgg tatttgaagg tctcctcccg ggtcagctcc accccgtcct | 2700 |
| tcagccattt gacttgcgcc ccctcctccg atacttcaca ctcaaactcc acccgctgcc | 2760 |
| ccaccatcac cagctggtcc tccaaggggc gcgtgatgag cacagggggc tctttcacaa | 2820 |
| agagctccgt gctacacttc tcgccaccca ccacgcactg gtaggctgcg tcgtccgcca | 2880 |
| atgagcactg gctgatggtc agggtacgct tggcaccgat ggactcaaag atgtacttgc | 2940 |
| tgccgctcat ctggatctcc tggccattct tgagccattt gacctcagcg tcatggtcag | 3000 |
| ccagttccac ggtcagccgg atcttgtggc ctttgctcac ctggtaggcc ggctccagct | 3060 |
| tcttctgaaa ggctgtgctc ttcttctcat cgcgcctcat gcccttgagc ctctttagca | 3120 |
| tgccgcgcag gtcagtgacg ccgtactgga aggcgatgcg ctcgtactca gatgggggtg | 3180 |
| cctgccgtag gatctcccac acgtcctcct ctgctggtgc ctccagcttc gagtccctcg | 3240 |
| gggtccggaa actgtctctc tttttcagca gtgagctgaa gtccagaatc ccagtgtcct | 3300 |
| catggctatc actgatccgc cgaccacctc cagccaggct cgtgcggcgg aaggctgata | 3360 |
| ggaggtccag gtctccggtg cccatggcct cgtggacagt gagattgaag ttggagcagt | 3420 |
| caaatttgtc cttggtggac acctcacagc ggtagctgcc agtgaaggca ggctgggcat | 3480 |
| cggtgatgtg cagctcgaac agatagacct tgctggcgcg gtcgtagctg tcgtgcagct | 3540 |
| gcaggtgctg gcccaccttg ctgctcaggt ccacccattt gcccttgaac cacttgacca | 3600 |
| caggcggctt caggaggctg gcgccggcca cgcgggctga gaaggtgatg ctgccaccca | 3660 |
| cggtcacctc gccatcctgt ggccgcatca cgaagaggcc aatggggtca tcgggggctc | 3720 |
| caggggtagg accattgaga gctgctgagc ttgacccttt gggacttggg gcactttctc | 3780 |
| ccagctcagc ggctggggcc ggggcttctc caggggctcc agtggcctca gcaggggcag | 3840 |
| gggcaggggc cagcatgggc tctgccttct ctgcctctat gaccttgagg tcgaacttga | 3900 |
| ccttggagga gccagcaatg actgcgtaag atccctggtc ggcagggccc acttcccgca | 3960 |
| ctgtcagcgt atgccgtgtg ccctctgtgg ccaggccgta cttgttgctg gcgctgatgt | 4020 |
| cactgcctcc gcgctgccag cgcaccttca ctcctgcccg ctctgtctcg gcctcgaaca | 4080 |
| cggcagggct gcctgcggcc acttccactg accgtggctt cttgctaaaa gctgagactg | 4140 |
| gcttcttccc cggctcaggc atcctgagag acgtcacacc aggcacgaag caggcacagg | 4200 |
| tcacccaaag agggact | 4217. |

Exemplary gRNA spacer sequences of the disclosure that specifically bind to a target sequence of an RNA molecule encoding a MYBPC3 protein of the disclosure may comprise or consist of a nucleic acid having a sequence selected from any one of any one of SEQ ID NO: 19801 to SEQ ID NO: 23992.

Compositions of the disclosure may comprise a gRNA comprising a spacer sequence that specifically binds to a target sequence of an RNA molecule encoding TNNT2 protein comprising or consisting of about 20-30 nucleotides of the sequence of:

| SEQ ID NO: 324) | |
|---|---|
| tcagtgtgtg gtggcttttt attactggtg tggagtgggt gtgggggcag gcaggagtgg | 60 |
| tggctcccac ctaggccagc tccccatttc caaacaggag ctgcctgggg tgcccaggag | 120 |
| ggcccgggaa ctgggggagt gcaggccgga ggcaggtgcg agcgaggagc agatctttgg | 180 |
| tgaaggaggc caggctctat ttccagcgcc cggtgacttt agccttcccg cgggtcttgg | 240 |
| agactttctg gttatcgttg atcctgtttc ggagaacatt gatctcatat ttctgctgct | 300 |
| tgaacttctc ctgcaggtcg aacttctctg cctccaagtt atagatgctc tgccacagct | 360 |
| ccttggcctt ctccctcagc tgatcttcat tcaggtggtc aatggccagc accttcctcc | 420 |
| tctcagccag aatcttcttc ttcttttccc gctcagtctg cctcttccca cttttccgct | 480 |
| ctgtctgggc ctgcttctgg atgtaacccc caaaatgcat catgttggac aaagccttct | 540 |
| tcttccgggc ctcatcctca gccttcctcc tgttctcctc ctcctctcgt cgagccctct | 600 |
| cttcagccag gcggttctgc cgctccttct cccgctcatt ccggatgcgc tgctgctcgg | 660 |
| cccgctctgc ccgacgtctc tcgatcctgt ctttgagaga aacgagctcc tcctcctctt | 720 |
| tcttcctgtt ctcaaagtga gcctcgatca gcgcctgcaa ctcattcagg tccttctcca | 780 |
| tgcgcttccg gtggatgtca tcaaagtcca ctctctctcc atcggggatc ttgggaggca | 840 |
| ccaagttggg catgaacgac ctgggctttg gtttggactc ctccattggg ccatcttcag | 900 |
| cctcctttgc ttcctcttct tcttcatctt cttctgccct ggtctcctcg gtctcagcct | 960 |
| ctgcttcagc atcctcttcc gctgcctcct cctgctcgtc ttcgtcctct ctccagtcct | 1020 |
| cctcttcttc aacagctgct tcttcctgct cctcctcctc gtactcttcc accacctctt | 1080 |
| ctatgtcaga catggtctct gctctccctc caaaaggaga aaaaagtcag tgcaggtaca | 1140 |
| aagggaagcc tgccttcctc agaagagctc tggcccccgt tgtacagaga tcagcgaggc | 1200 |
| ctagggtgaa tctagttcca cccctcatga gctgtgtgac ctcagaacag cagctgccga | 1260 |
| cagatcctgg aggcgtctgc tcagtctcag cggggactgg gtgaggcaga ggatggagag | 1320 |
| ggctttaagc aggcatgtgg gctggggcct ggtgagccag cc | 1362. |

Exemplary gRNA spacer sequences of the disclosure that specifically bind to a target sequence of an RNA molecule encoding a TNNT2 protein of the disclosure may comprise or consist of a nucleic acid having a sequence selected from any one of any one of SEQ ID NO: 23993 to SEQ ID NO: 25329.

Compositions of the disclosure may comprise a gRNA comprising a spacer sequence that specifically binds to a target sequence of an RNA molecule encoding pre-mRNA processing factor 31 (PRPF31) protein comprising or consisting of about 20-30 nucleotides of the sequence of:

| (SEQ ID NO: 491) | |
|---|---|
| tcttgacaat gtccttttaa ttgtactctt ttcaaaaaat ctcctttctc agttaaaaaa | 60 |
| gacaaggcat gatgaagacc tgctctagcc catactgggc ggtgatctcg gtcctggggg | 120 |
| aggccaggcc ggactcttcc aaggcctcct ccctgggcag tcccagcaat ggggccagtg | 180 |
| gcagggcagg ttctccctgc cagaacccga tcctagccct tcagaaggac tggacctctg | 240 |
| tgtcccttca gtgggaagcc accttggaca cacgcagtca ttcaggtgga cataaggcca | 300 |
| ctcttctcgc ccttgacctt gaggaactca gccatgctgg agaaatactt ctggttggcc | 360 |
| tcagccacct tcttctctgc cgcctgtggg ttcacaatct ccaggccctg gagtggggtg | 420 |
| aaggccacgc tggaggccgt gcccgaggag cggtcgcgga tggtggactt cccgccatat | 480 |
| acgacgctct gcttctgcag ggtccgctgc agcgtcttgg agatcctggc cttggtggcc | 540 |
| tcgtttacct gtgtctgccg cacacgccca ctgcccgact tgcccaggtg gcccaggctg | 600 |
| aatcccaggt cctcctggta ggcgtcctcc tcgatctctc cgaagctcat acggttggcc | 660 |
| tgcttccgga tctccgtcag ccccagccgc tccttcatct tgcggtacct gcggccgcct | 720 |
| cgcttcttcc gctgtccatc caggggcgca ggcagcggct tcacctgctt cacaggcggc | 780 |
| ggctcctgcc acttgtcgaa tttgcgctcg atctcatcct tcagttcgta gcccaccttc | 840 |
| ccttctgtgc tctcgtggaa actgtccaca cgggctgcca gtgtgcactt ggcggccacc | 900 |
| agccgggccg ctttccgccg cagatccggt ggcagggact gcacgatgtc actgtggtag | 960 |
| atgtagccgg tgtggggcag cactgaggta gacgagaagc ccgacagcgt cttgcgctgg | 1020 |
| gccccgagca gcatgatgtt gcaggcgggc atcttggaga ggttggtcag gccgccggcc | 1080 |
| acacccatga tcttggcggc cgtggatgcc ccgataatga tggacaggtt gggtgcgatg | 1140 |
| aaggacatcc gggactccac atactcgtag atgcggtgct tggaggcgtt cagctccagc | 1200 |
| gccatgtcgc aggcctcctc cagccgctcc agctcctcct ccgacagctg ctgcccctgg | 1260 |
| gtggtggagg cggtgacgct gacgaccatg atggtggcat tggtgaggat ctgctgcagg | 1320 |
| ttctcattgt tcttgcactt gtccaggctg ttgcccagct ccttgaccgt gcggatgtaa | 1380 |
| tccagtgcat tggggaccaa ggactccagt tcagggaatc tctttgagta cttatcccgg | 1440 |
| atgaacttat ggatgatgtt cagctcgttt tcgatctcca cggtcaggtt gttggcatcc | 1500 |
| acgatgacgc ggtattcagg cgcggcctcc actggtccca tcacttctga agctttggct | 1560 |
| tgcttgctga tatactcctc aatcttcatc ataatctcag caaacatctt actatcccat | 1620 |
| agcttggcga tggtcttgac tgaatccccg gaaagatcca gctgtgtctc ctcctgcaca | 1680 |
| tcctcgatcg ctggctcctc ttcttcctcc ccatagcttc ctccttcctc ctcttctgct | 1740 |
| gcctcttcga gatcagctaa gagctcatct gccagagaca tcccgaggcc tctcctctcc | 1800 |
| gcgcaccact gtttctagcg ttagtcgctc acc | 1833. |

Exemplary gRNA spacer sequences of the disclosure that specifically bind to a target sequence of an RNA molecule encoding a PRPF31 protein of the disclosure may comprise or consist of a nucleic acid having a sequence selected from any one of any one of SEQ ID NO: 25330 to SEQ ID NO: 27137.

Compositions of the disclosure may comprise a gRNA comprising a spacer sequence that specifically binds to a target sequence of an RNA molecule encoding Progranulin (GRN) protein comprising or consisting of about 20-30 nucleotides of the sequence of:

| (SEQ ID NO: 492) | |
|---|---|
| ttaagaaagt gtacaaactt tattgaaacg cacacgcgca cacacacaaa cacccctgtg | 60 |
| gatagggaaa agcacctggc cacagggtcc actgaaacgg ggaggggatg gcagcttgta | 120 |
| atgtggcttt tgccacaacc cccttctgac agggaaggcc ttagattgag gccccacctc | 180 |
| ccatggtgat ggggagctca gaatggggtc cagggagaat ttggttaggg ggaggtgcta | 240 |
| gggaggcctg agcagagggc accctccgag tggggtcccg agggctgcag agtcttcagt | 300 |
| actgtccctc acagcagctg tctcaaggct gggtccctca aaggggcgtc ccagcgcggg | 360 |
| gcctccctgc gcaaacactt ggtacccctg gctgcgcagc ggaagccagc aggacagcag | 420 |
| tggcgccgat cagcacaaca gacgccctgg cggtagggac agcaggccca gccctgtcgg | 480 |
| ttgtctcggc agcaggtctg gttatcatgg cagaagtgtc cttccccaca ctccacgtcc | 540 |
| ttcacaccca cgtgagggct acgggccagg aaggtggcag gctgggcaga gaccacttcc | 600 |
| ttctcgcagg atcgagcctt cacgttgcag gtgtagccag ccgggcagca gtgctggcga | 660 |
| tcctcgcagc acacagcatg gggcaactgg cagcaggccc agctcccacc caggctcggg | 720 |
| cagcaggtct gccccaccgg gcagctggtg tgctggtcac agccgatgtc tctggggtgg | 780 |
| gataaggaag cccggcgggc aggcatcttc tccagtccag ccacgatctc gcttcctcgc | 840 |
| tgacactgcc cctcagctac acacgtgtag ccctgggggc agcagtgctg gtggtccgag | 900 |
| cagcagacag cctctgggat tggacagcag ccccactccc cagacgtgag ttggcagcag | 960 |
| gtatcggagg agggacagct gctgacatta tcacagggga catctctctt caaggcttgt | 1020 |
| gggtctggca ggctgaggtg agctggggcc ttctccatcc agggcacctg gtggggcccc | 1080 |
| tgttcacagg tacccttctg cgtgtcacac gtaaaccccg cgggacagca gtgtatgtgg | 1140 |
| tcctcacagc acacagcctg ggtaaaaggg cagcagcccc aggcccccga ctgtagacgg | 1200 |
| cagcaggtat agccatctgg gcagctcacc tccatgtcac atttcacatc ccccactgtg | 1260 |
| tgcgcaggca gcttagtgag gaggtccgtg gtagcgttct ccttggagag gcacttactc | 1320 |
| tggatcaggt cacacacagt gtcttggggg cagcagtgca ggtgatcgga gcagcaggtg | 1380 |
| gcgttgggca ttgggcagca gccatacttc ccactgggca gctcacagca ggtagaacca | 1440 |
| tcagggcacc gggaccgtgc gtccggacac atgaccgagc tggacaaggc cactgccctg | 1500 |
| ttagtcctct gggcagggag cttctttgcc agggggtggg tgcccgtggg tgtgatgcag | 1560 |
| cgggtgtgaa ccaggtcgca gaaggcaccg tgcggacagc agtgcaccct gtcttcacag | 1620 |
| caggaagcct ggggcatggg gcagcacccc caggagccat cgaccataac acagcacgtg | 1680 |
| gagaagtccg ggcattcgaa ctgactatca gggcactgga tggcacccac ggagttgtta | 1740 |
| cctgatcttt ggaagcagga tcgcccgtct gcactgcagt ggaagccccg tgggcagcag | 1800 |
| tgatggccat ccccgcatgc cacggcctct gggaaggggc agcaactgga agtccctgag | 1860 |
| acggtaaaga tgcaggagtg gccggcagag cagtgggcat caacctggca ggggccaccc | 1920 |
| agatgcctgc tcagtgttgt gggccatttg tccagaaggg gacggcagca gctgtagctg | 1980 |
| gctcctccgg ggtccaggca gcaggccaca gggcagaact gaccatctgg gcaccgcgtt | 2040 |
| ccagccacca gccctgctgt taaggccacc cagctcacca gggtccacat ggtctgcctg | 2100 |
| cgtccgactc cgcggtcctt gggcagcagc | 2130. |

Exemplary gRNA spacer sequences of the disclosure that specifically bind to a target sequence of an RNA molecule encoding a Progranulin (GRN) protein of the disclosure may comprise or consist of a nucleic acid having a sequence selected from any one of any one of SEQ ID NO: 27138 to SEQ ID NO: 29242.

### RNA Molecules

In some embodiments of the compositions and methods of the disclosure, an RNA molecule of the disclosure comprises a target RNA sequence. In some embodiments, a pathogenic RNA comprises the target RNA sequence or the target sequence is associated with the pathogenic RNA. In some embodiments, the RNA molecule of the disclosure comprises at least one target sequence. In some embodiments, the RNA molecule of the disclosure comprises one or more target sequence(s). In some embodiments, the RNA molecule of the disclosure comprises two or more target sequences. In some embodiments the target RNA is non-coding RNA.

In some embodiments of the compositions and methods of the disclosure, an RNA molecule of the disclosure is a naturally occurring RNA molecule. In some embodiments, the RNA molecule of the disclosure is a non-naturally occurring molecule. Exemplary non-naturally occurring RNA molecules may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, an RNA molecule of the disclosure comprises or consists of a sequence isolated or derived from a virus.

In some embodiments of the compositions and methods of the disclosure, an RNA molecule of the disclosure comprises or consists of a sequence isolated or derived from a prokaryotic organism. In some embodiments, an RNA molecule of the disclosure comprises or consists of a sequence isolated or derived from a species or strain of archaea or a species or strain of bacteria.

In some embodiments of the compositions and methods of the disclosure, the RNA molecule of the disclosure comprises or consists of a sequence isolated or derived from a eukaryotic organism. In some embodiments, an RNA molecule of the disclosure comprises or consists of a sequence isolated or derived from a species of protozoa, parasite, protist, algae, fungi, yeast, amoeba, worm, microorganism, invertebrate, vertebrate, insect, rodent, mouse, rat, mammal, or a primate. In some embodiments, an RNA molecule of the disclosure comprises or consists of a sequence isolated or derived from a human.

In some embodiments of the compositions and methods of the disclosure, the RNA molecule of the disclosure comprises or consists of a sequence derived from a coding sequence from a genome of an organism or a virus. In some embodiments, the RNA molecule of the disclosure comprises or consists of a primary RNA transcript, a precursor messenger RNA (pre-mRNA) or messenger RNA (mRNA). In some embodiments, the RNA molecule of the disclosure comprises or consists of a gene product that has not been processed (e.g. a transcript). In some embodiments, the RNA molecule of the disclosure comprises or consists of a gene product that has been subject to post-transcriptional processing (e.g. a transcript comprising a 5′cap and a 3′ polyadenylation signal). In some embodiments, the RNA molecule of the disclosure comprises or consists of a gene product that has been subject to alternative splicing (e.g. a splice variant). In some embodiments, the RNA molecule of the disclosure comprises or consists of a gene product that has been subject to removal of non-coding and/or intronic sequences (e.g. a messenger RNA (mRNA)).

In some embodiments of the compositions and methods of the disclosure, the RNA molecule of the disclosure comprises or consists of a sequence derived from a non-coding sequence (e.g. a non-coding RNA (ncRNA)). In some embodiments, the RNA molecule of the disclosure comprises or consists of a ribosomal RNA. In some embodiments, the RNA molecule of the disclosure comprises or consists of a small ncRNA molecule. Exemplary small RNA molecules of the disclosure include, but are not limited to, microRNAs (miRNAs), small interfering (siRNAs), piwi-interacting RNAs (piRNAs), small nucleolar RNAs (snoRNAs), small nuclear RNAs (snRNAs), extracellular or exosomal RNAs (exRNAs), and small Cajal body-specific RNAs (scaRNAs). In some embodiments, the RNA molecule of the disclosure comprises or consists of a long ncRNA molecule. Exemplary long RNA molecules of the disclosure include, but are not limited to, X-inactive specific transcript (Xist) and HOX transcript antisense RNA (HOTAIR).

In some embodiments of the compositions and methods of the disclosure, the RNA molecule of the disclosure contacted by a composition of the disclosure in an intracellular space. In some embodiments, the RNA molecule of the disclosure contacted by a composition of the disclosure in a cytosolic space. In some embodiments, the RNA molecule of the disclosure contacted by a composition of the disclosure in a nucleus. In some embodiments, the RNA molecule of the disclosure contacted by a composition of the disclosure in a vesicle, membrane-bound compartment of a cell, or an organelle.

In some embodiments of the compositions and methods of the disclosure, the RNA molecule of the disclosure contacted by a composition of the disclosure in an extracellular space. In some embodiments, the RNA molecule of the disclosure contacted by a composition of the disclosure in an exosome. In some embodiments, the RNA molecule of the disclosure contacted by a composition of the disclosure in a liposome, a polymersome, a micelle or a nanoparticle. In some embodiments, the RNA molecule of the disclosure contacted by a composition of the disclosure in an extracellular matrix. In some embodiments, the RNA molecule of the disclosure contacted by a composition of the disclosure in a droplet. In some embodiments, the RNA molecule of the disclosure contacted by a composition of the disclosure in a microfluidic droplet.

In some embodiments of the compositions and methods of the disclosure, a RNA molecule of the disclosure comprises or consists of a single-stranded sequence. In some embodiments, the RNA molecule of the disclosure comprises or consists of a double-stranded sequence. In some embodiments, the double-stranded sequence comprises two RNA molecules. In some embodiments, the double-stranded sequence comprises one RNA molecule and one DNA molecule. In some embodiments, including those wherein the double-stranded sequence comprises one RNA molecule and one DNA molecule, compositions of the disclosure selectively bind and, optionally, selectively cut the RNA molecule.

### RNA-Binding Endonucleases

In some embodiments of the compositions of the disclosure, there may be an optional second RNA binding protein which comprises or consists of a nuclease or endonuclease domain. In some embodiments, the second RNA-binding protein is an effector protein. In some embodiments, the second RNA binding protein binds RNA in a manner in which it associates with RNA. In some embodiments, the second RNA binding protein associates with RNA in a manner in which it cleaves RNA. In some embodiments, the second RNA-binding protein is fused to a first RNA-binding protein which is a PUF, PUMBY, or PPR-based protein.

In some embodiments of the compositions of the disclosure, the second RNA binding protein comprises or consists of an RNAse.

In some embodiments, the second RNA binding protein comprises or consists of an RNAse1. In some embodiments, the RNAse1 protein comprises or consists of SEQ ID NO: 325.

In some embodiments, the second RNA binding protein comprises or consists of an RNAse4. In some embodiments, the RNAse4 protein comprises or consists of SEQ ID NO: 326.

In some embodiments, the second RNA binding protein comprises or consists of an RNAse6. In some embodiments, the RNAse6 protein comprises or consists of SEQ ID NO: 327.

In some embodiments, the second RNA binding protein comprises or consists of an RNAse7. In some embodiments, the RNAse7 protein comprises or consists of SEQ ID NO: 328.

In some embodiments, the second RNA binding protein comprises or consists of an RNAse8. In some embodiments, the RNAse8 protein comprises or consists of SEQ ID NO: 329.

In some embodiments, the second RNA binding protein comprises or consists of an RNAse2. In some embodiments, the RNAse2 protein comprises or consists of SEQ ID NO: 330.

In some embodiments, the second RNA binding protein comprises or consists of an RNAse6PL. In some embodiments, the RNAse6PL protein comprises or consists of SEQ ID NO: 331.

In some embodiments, the second RNA binding protein comprises or consists of an RNAseL. In some embodiments, the RNAseL protein comprises or consists of SEQ ID NO: 332.

In some embodiments, the second RNA binding protein comprises or consists of an RNAseT2. In some embodiments, the RNAseT2 protein comprises or consists of SEQ ID NO: 333.

In some embodiments, the second RNA binding protein comprises or consists of an RNAse11. In some embodiments, the RNAse11 protein comprises or consists of SEQ ID NO: 334.

In some embodiments, the second RNA binding protein comprises or consists of an RNAseT2-like. In some embodiments, the RNAseT2-like protein comprises or consists of SEQ ID NO: 335.

In some embodiments of the compositions of the disclosure, the second RNA binding protein comprises or consists of a mutated RNAse.

In some embodiments, the second RNA binding protein comprises or consists of a mutated Rnase1 (Rnase1(K41R)) polypeptide. In some embodiments, the Rnase1(K41R) polypeptide comprises or consists of SEQ ID NO: 336.

In some embodiments, the second RNA binding protein comprises or consists of a mutated Rnase1 (Rnase1(K41R, D121E)) polypeptide. In some embodiments, the Rnase1 (Rnase1(K41R, D121E)) polypeptide comprises or consists of SEQ ID NO: 337.

In some embodiments, the second RNA binding protein comprises or consists of a mutated Rnase1 (Rnase1(K41R, D121E, H119N)) polypeptide. In some embodiments, the Rnase1 (Rnase1(K41R, D121E, H119N)) polypeptide comprises or consists of SEQ ID NO: 338.

In some embodiments, the second RNA binding protein comprises or consists of a mutated Rnase1. In some embodiments, the second RNA binding protein comprises or consists of a mutated Rnase1 (Rnase1(H119N)) polypeptide. In some embodiments, the Rnase1 (Rnase1(H119N)) polypeptide comprises or consists of SEQ ID NO: 339.

In some embodiments, the second RNA binding protein comprises or consists of a mutated Rnase1 (Rnase1(R39D, N67D, N88A, G89D, R91D, H19N)) polypeptide.

In some embodiments, the Rnase1 (Rnase1(R39D, N67D, N88A, G89D, R91D, H119N)) polypeptide comprises or consists of SEQ ID NO: 340.

In some embodiments, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D, H119N, K41R, D121E)) polypeptide comprises or consists of SEQ ID NO: 341.

In some embodiments, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D)) polypeptide comprises or consists of SEQ ID NO: 342.

In some embodiments, the second RNA binding protein comprises or consists of a mutated Rnase1 (Rnase1 (R39D, N67D, N88A, G89D, R91D, H119N, K41R, D121E)) polypeptide that comprises or consists of SEQ ID NO: 343.

In some embodiments of the compositions of the disclosure, the second RNA binding protein comprises or consists of a NOB1 polypeptide. In some embodiments, the NOB1 polypeptide comprises or consists of SEQ ID NO: 344.

In some embodiments of the compositions of the disclosure, the second RNA binding protein comprises or consists of an endonuclease. In some embodiments, the second RNA binding protein comprises or consists of an endonuclease V (ENDOV). In some embodiments, the ENDOV protein comprises or consists of SEQ ID NO: 345.

In some embodiments, the second RNA binding protein comprises or consists of an endonuclease G (ENDOG). In some embodiments, the ENDOG protein comprises or consists of SEQ ID NO: 346.

In some embodiments, the second RNA binding protein comprises or consists of an endonuclease D1 (ENDOD1). In some embodiments, the ENDOD1 protein comprises or consists of SEQ ID NO: 347.

In some embodiments, the second RNA binding protein comprises or consists of a Human flap endonuclease-1 (hFEN1). In some embodiments, the hFEN1 polypeptide comprises or consists of SEQ ID NO: 348.

In some embodiments, the second RNA binding protein comprises or consists of a DNA repair endonuclease XPF (ERCC4) polypeptide. In some embodiments, the ERCC4 polypeptide comprises or consists of SEQ ID NO: 349.

In some embodiments of the compositions of the disclosure, the second RNA binding protein comprises or consists of an Endonuclease III-like protein 1 (NTHL) polypeptide. In some embodiments, the NTHL polypeptide comprises or consists of SEQ ID NO: 340.

In some embodiments of the compositions of the disclosure, the second RNA binding protein comprises or consists of a human Schlafen 14 (hSLFN14) polypeptide. In some embodiments, the hSLFN14 polypeptide comprises or consists of SEQ ID NO: 351.

In some embodiments of the compositions of the disclosure, the second RNA binding protein comprises or consists of a human beta-lactamase-like protein 2 (hLACTB2) polypeptide. In some embodiments, the hLACTB2 polypeptide comprises or consists of SEQ ID NO: 352.

In some embodiments of the compositions of the disclosure, the second RNA binding protein comprises or consists of an apurinic/apyrimidinic (AP) endodeoxyribonuclease (APEX) polypeptide. In some embodiments, the second RNA binding protein comprises or consists of an apurinic/apyrimidinic (AP) endodeoxyribonuclease (APEX2) polypeptide. In some embodiments, the APEX2 polypeptide comprises or consists of SEQ ID NO: 353.

In some embodiments, the APEX2 polypeptide comprises or consists of SEQ ID NO: 354.

In some embodiments, the second RNA binding protein comprises or consists of an apurinic or apyrimidinic site lyase (APEX1) polypeptide. In some embodiments, the APEX1 polypeptide comprises or consists of SEQ ID NO: 355.

In some embodiments of the compositions of the disclosure, the second RNA binding protein comprises or consists of an angiogenin (ANG) polypeptide. In some embodiments, the ANG polypeptide comprises or consists of SEQ ID NO: 356.

In some embodiments of the compositions of the disclosure, the second RNA binding protein comprises or consists of a heat responsive protein 12 (HRSP12) polypeptide. In some embodiments, the HRSP12 polypeptide comprises or consists of SEQ ID NO: 357.

In some embodiments of the compositions of the disclosure, the second RNA binding protein comprises or consists of a Zinc Finger CCCH-Type Containing 12A (ZC3H12A) polypeptide. In some embodiments, the ZC3H12A polypeptide comprises or consists of SEQ ID NO: 358.

In some embodiments, the ZC3H12A polypeptide comprises or consists of SEQ ID NO: 359.

In some embodiments of the compositions of the disclosure, the second RNA binding protein comprises or consists of a Reactive Intermediate Imine Deaminase A (RIDA) polypeptide. In some embodiments, the RIDA polypeptide comprises or consists of SEQ ID NO: 360.

In some embodiments of the compositions of the disclosure, the second RNA binding protein comprises or consists of a Phospholipase D Family Member 6 (PDL6) polypeptide. In some embodiments, the PDL6 polypeptide comprises or consists of SEQ ID NO: 361.

In some embodiments of the compositions of the disclosure, the second RNA binding protein comprises or consists of a mitochondrial ribonuclease P catalytic subunit (KIAA0391) polypeptide. In some embodiments, the KIAA0391 polypeptide comprises or consists of SEQ ID NO: 362.

In some embodiments of the compositions of the disclosure, the second RNA binding protein comprises or consists of an argonaute 2 (AGO2) polypeptide.

In some embodiments of the compositions of the disclosure, the AGO2 polypeptide comprises or consists of SEQ ID NO: 363.

In some embodiments of the compositions of the disclosure, the second RNA binding protein comprises or consists of a mitochondrial nuclease EXOG (EXOG) polypeptide. In some embodiments, the EXOG polypeptide comprises or consists of SEQ ID NO: 364.

In some embodiments of the compositions of the disclosure, the second RNA binding protein comprises or consists of a Zinc Finger CCCH-Type Containing 12D (ZC3H12D) polypeptide. In some embodiments, the ZC3H12D polypeptide comprises or consists of SEQ ID NO: 365.

In some embodiments of the compositions of the disclosure, the second RNA binding protein comprises or consists of an endoplasmic reticulum to nucleus signaling 2 (ERN2) polypeptide. In some embodiments, the ERN2 polypeptide comprises or consists of SEQ ID NO: 366.

In some embodiments of the compositions of the disclosure, the second RNA binding protein comprises or consists of a pelota mRNA surveillance and ribosome rescue factor (PELO) polypeptide. In some embodiments, the PELO polypeptide comprises or consists of SEQ ID NO: 367.

In some embodiments of the compositions of the disclosure, the second RNA binding protein comprises or consists of a YBEY metallopeptidase (YBEY) polypeptide. In some embodiments, the YBEY polypeptide comprises or consists of SEQ ID NO: 368.

In some embodiments of the compositions of the disclosure, the second RNA binding protein comprises or consists of a cleavage and polyadenylation specific factor 4 like (CPSF4L) polypeptide. In some embodiments, the CPSF4L polypeptide comprises or consists of SEQ ID NO: 369.

In some embodiments of the compositions of the disclosure, the second RNA binding protein comprises or consists of an hCG_2002731 polypeptide. In some embodiments, the hCG_2002731 polypeptide comprises or consists of SEQ ID NO: 370.

In some embodiments, the hCG_2002731 polypeptide comprises or consists of SEQ ID NO: 371.

In some embodiments of the compositions of the disclosure, the second RNA binding protein comprises or consists of an Excision Repair Cross-Complementation Group 1 (ERCC1) polypeptide. In some embodiments, the ERCC1 polypeptide comprises or consists of SEQ ID NO: 372.

In some embodiments of the compositions of the disclosure, the second RNA binding protein comprises or consists of a ras-related C3 botulinum toxin substrate 1 isoform (RAC1) polypeptide. In some embodiments, the RAC1 polypeptide comprises or consists of SEQ ID NO: 373.

In some embodiments of the compositions of the disclosure, the second RNA binding protein comprises or consists of a Ribonuclease A A1 (RAA1) polypeptide. In some embodiments, the RAA1 polypeptide comprises or consists of SEQ ID NO: 374.

In some embodiments of the compositions of the disclosure, the second RNA binding protein comprises or consists of a Ras Related Protein (RAB1) polypeptide. In some embodiments, the RAB1 polypeptide comprises or consists of SEQ ID NO: 375.

In some embodiments of the compositions of the disclosure, the second RNA binding protein comprises or consists of a DNA Replication Helicase/Nuclease 2 (DNA2) polypeptide. In some embodiments, the DNA2 polypeptide comprises or consists of SEQ ID NO: 376.

In some embodiments of the compositions of the disclosure, the second RNA binding protein comprises or consists of a FLJ35220 polypeptide. In some embodiments, the FLJ35220 polypeptide comprises or consists of SEQ ID NO: 377.

In some embodiments of the compositions of the disclosure, the second RNA binding protein comprises or consists of a FLJ13173 polypeptide. In some embodiments, the FLJ13173 polypeptide comprises or consists of SEQ ID NO: 378.

In some embodiments of the compositions of the disclosure, the second RNA binding protein comprises or consists of Teneurin Transmembrane Protein (TENM) polypeptide. In some embodiments, the second RNA binding protein comprises or consists of Teneurin Transmembrane Protein 1 (TENM1) polypeptide. In some embodiments, the TENM1 polypeptide comprises or consists of SEQ ID NO: 379.

In some embodiments, the second RNA binding protein comprises or consists of Teneurin Transmembrane Protein 2 (TENM2) polypeptide. In some embodiments, the TENM2 polypeptide comprises or consists of SEQ ID NO: 380.

In some embodiments of the compositions of the disclosure, the second RNA binding protein comprises or consists of a Ribonuclease Kappa (RNAseK) polypeptide. In some embodiments, the RNAseK polypeptide comprises or consists of SEQ ID NO: 381.

In some embodiments of the compositions of the disclosure, the second RNA binding protein comprises or consists of a transcription activator-like effector nuclease (TALEN) polypeptide or a nuclease domain thereof. In some embodiments, the TALEN polypeptide comprises or consists of SEQ ID NO: 382. In some embodiments, the TALEN polypeptide comprises or consists of SEQ ID NO: 383.

In some embodiments of the compositions of the disclosure, the second RNA binding protein comprises or consists a zinc finger nuclease polypeptide or a nuclease domain thereof. In some embodiments, the second RNA binding protein comprises or consists of a ZNF638 polypeptide or a nuclease domain thereof. In some embodiments, the ZNF638 polypeptide polypeptide comprises or consists of SEQ ID NO: 384.

In some embodiments of the compositions of the disclosure, the second RNA binding protein comprises or consists of a PIN domain derived from the human SMG6 protein, also commonly known as telomerase-binding protein EST1A isoform 3, NCBI Reference Sequence: NP_001243756.1. In some embodiments, the PIN from hSMG6 is used herein in the form of a Cas fusion protein and as an internal control, for example, and without limitation, see FIG. 9, which shows PIN-dSauCas9, PIN-dSauCas9dHNH, PIN-dSPCas9, and dcjeCas9-PIN.

In some embodiments of the compositions of the disclosure, the composition further comprises (a) a sequence comprising a gRNA that specifically binds within an RNA molecule and (b) a sequence encoding a nuclease. In some embodiments, a nuclease comprises a sequence isolated or derived from a CRISPR/Cas protein. In some embodiments, the CRISPR/Cas protein is isolated or derived from any one of a type I, a type IA, a type IB, a type IC, a type ID, a type IE, a type IF, a type IU, a type III, a type IIIA, a type IIIB, a type IIIC, a type IIID, a type IV, a type IVA, a type IVB, a type II, a type IIA, a type IIB, a type IIC, a type V, or a type VI CRISPR/Cas protein. In some embodiments, a nuclease comprises a sequence isolated or derived from a TALEN or a nuclease domain thereof. In some embodiments, a nuclease comprises a sequence isolated or derived from a zinc finger nuclease or a nuclease domain thereof.

### Fusion Proteins

In some embodiments of the compositions and methods of the disclosure, the composition comprises a sequence encoding a target RNA-binding fusion protein comprising (a) a sequence encoding a first RNA-binding polypeptide or portion thereof, and optionally (b) a sequence encoding a second RNA-binding polypeptide, wherein the first RNA-biding polypeptide binds a target RNA, and wherein the second RNA-binding polypeptide comprises RNA-nuclease activity.

In some embodiments, a target RNA-binding fusion protein is an RNA-guided target RNA-binding fusion protein. RNA-guided target RNA-binding fusion proteins comprise at least one RNA-binding polypeptide which corresponds to a gRNA which guides the RNA-binding polypeptide to target RNA. RNA-guided target RNA-binding fusion proteins include without limitation, RNA-binding polypeptides which are CRISPR/Cas-based RNA-binding polypeptides or portions thereof.

In some embodiments, a target RNA-binding fusion protein is not an RNA-guided target RNA-binding fusion protein and as such comprises at least one RNA-binding polypeptide which is capable of binding a target RNA without a corresponding gRNA sequence. Such non-guided RNA-binding polypeptides include, without limitation, at least one RNA-binding protein or RNA-binding portion thereof which is a PUF (Pumilio and FBF homology family). This type RNA-binding polypeptide can be used in place of a gRNA-guided RNA binding protein such as CRISPR/Cas. The unique RNA recognition mode of PUF proteins (named for Drosophila Pumilio and C. elegans fem-3 binding factor) that are involved in mediating mRNA stability and translation are well known in the art. The PUF domain of human Pumiliol, also known in the art, binds tightly to cognate RNA sequences and its specificity can be modified. It contains eight PUF repeats that recognize eight consecutive RNA bases with each repeat recognizing a single base. Since two amino acid side chains in each repeat recognize the Watson-Crick edge of the corresponding base and determine the specificity of that repeat, a PUF domain can be designed to specifically bind most 8-nt RNA. Wang et al., Nat Methods. 2009; 6(11): 825-830. See also WO2012/068627 which is incorporated by reference herein in its entirety.

The modular nature of the PUF-RNA interaction has been used to rationally engineer the binding specificity of PUF domains (Cheong, C. G. & Hall, T. M. (2006) PNAS 103: 13635-13639; Wang, X. et al (2002) Cell 110: 501-512). However, only the successful design of PUF domains with repeats that recognize adenine, guanine or uracil have been reported prior to the teachings of WO2012/06827 supra. While the wild-type PumHD does not bind C, molecular engineering has shown that some of the Pum units can be mutated to bind C with good yield and specificity. See e.g., Dong, S. et al. Specific and modular binding code for cytosine recognition in Pumilio/FBF (PUF) RNA-binding domains, The Journal of biological chemistry 286, 26732-26742 (2011). Accordingly, PumHD is a modified version of the WT Pumilio protein that exhibits programmable binding to arbitrary 8-base sequences of RNA. Each of the eight units of PumHD can bind to all four RNA bases, and the RNA bases flanking the target sequence do not affect binding. See also the following for art-recognized RNA-binding rules of PUF design: Filipovska A, Razif M F, Nygørd KK, & Rackham O. A universal code for RNA recognition by PUF proteins. Nature chemical biology, 7(7), 425-427 (2011); Filipovska A, & Rackham O. Modular recognition of nucleic acids by PUF, TALE and PPR proteins. Molecular BioSystems, 8(3), 699-708 (2012); Abil Z, Denard C A, & Zhao H. Modular assembly of designer PUF proteins for specific post-transcriptional regulation of endogenous RNA. Journal of biological engineering, 8(1), 7 (2014); Zhao Y, Mao M, Zhang W, Wang J, Li H, Yang Y, Wang Z, & Wu J. Expanding RNA binding specificity and affinity of engineered PUF domains. Nucleic Acids Research, 46(9), 4771-4782 (2018); Shinoda K, Tsuji S, Futaki S, & Imanishi M. Nested PUF Proteins: Extending Target RNA Elements for Gene Regulation. ChemBioChem, 19(2), 171-176 (2018); Koh Y Y, Wang Y, Qiu C, Opperman L, Gross L, Tanaka Hall T M, & Wickens M. Stacking Interactions in PUF-RNA Complexes. RNA, 17(4), 718-727 (2011).

As such, it is well known in the art that human PUM1 (1186 amino acids) contains an RNA-binding domain (RBD) in the C-terminus of the protein (also known as Pumilio homology domain PUM-HD amino acid 828-amino acid 1175) and that PUFs are based on the RBD of human PUM1. There are 8 structural repeat modules of 36 amino acids (except module 7 has 43 amino acids) for RNA binding and flanking N- and C-terminal regions important for protein structure and stability. Within each repeat module, amino acids 12, 13, and 16 are important for RNA binding with 12 and 16 controlling RNA base recognition. Amino acid 13 stacks with RNA bases and can be modified to tune specificity and affinity. Alternatively, the PUF design may maintain amino acid 13 as human PUM1's native residue. Recognition occurs in reverse orientation as N- and C-terminal PUF recognizes 3′ to 5′ RNA. Accordingly, PUF engineering of 8 modules (8PUF), as known in the art, mimics a human protein. An exemplary 8-mer RNA recognition (8PUF) would designed as follows: R1′-R1-R2-R3-R4-R5-R6-R7-R8-R8′. In one embodiment, an 8PUF is used as the RBD. In another embodiment, a variation of the 8PUF design is used to create a 12-mer RNA recognition (12PUF) RBD or a 16-mer RNA recognition (16PUF) RBD. Repeats 1-8 of wild type human PUM1 are provided herewith at SEQ ID NOS: 609-616, respectively. The nucleic acid sequence encoding the PUF domain from human PUM1 is SEQ ID NO: 617 and the amino acid sequence of the PUF domain from human PUM1 amino acids 828-176 is SEQ ID NO: 618. See also U.S. Pat. No. 9,580,714 which is incorporated herein in its entirety.

In some embodiments of the non-guided RNA-binding fusion proteins of the disclosure, the fusion protein comprises at least one RNA-binding protein or RNA-binding portion thereof which is a PUMBY (Pumilio-based assembly) protein. RNA-binding protein PumHD, which has been widely used in native and modified form for targeting RNA, has been engineered into a protein architecture designed to yield a set of four canonical protein modules, each of which targets one RNA base. These modules (i.e., Pumby, for Pumilio-based assembly) are concatenated in chains of varying composition and length, to bind desired target RNAs. In essence, PUMBY is a more simple and modular form of PumHD, in which a single protein unit of PumHD is concatenated into arrays of arbitrary size and binding sequence specificity. The specificity of such Pumby-RNA interactions is high, with undetectable binding of a Pumby chain to RNA sequences that bear three or more mismatches from the target sequence. Katarzyna et al., PNAS, 2016; 113(19): E2579-E2588. See also US 2016/0238593 which is incorporated by reference herein in its entirety.

In some embodiments of the compositions of the disclosure, the first RNA binding protein comprises a Pumilio and FBF (PUF) protein. In some embodiments, the first RNA binding protein comprises a Pumilio-based assembly (PUMBY) protein. In some embodiments, the PUF or PUMBY RNA-binding proteins are fused with a nuclease domain such as E17.

Exemplary PUF RNA-binding protein used in the compositions and methods disclosed herein are as follows:

In some embodiments, a PUF26 protein (original sequence) of the disclosure comprises or consists of the amino acid sequence of SEQ ID NO: 393.

In some embodiments, a PUF26 protein of the disclosure is encoded by an optimized nucleic acid sequence comprising or consisting of SEQ ID NO: 394.

In some embodiments, a PUF54 protein (original sequence) of the disclosure comprises or consists of the amino acid sequence of SEQ ID NO: 395.

In some embodiments, a PUF54 protein of the disclosure is encoded by an optimized nucleic acid sequence comprising or consisting of SEQ ID NO: 396.

In some embodiments, a PUF60 protein (original sequence) of the disclosure comprises or consists of the amino acid sequence of SEQ ID NO: 397.

In some embodiments, a PUF60 protein of the disclosure is encoded by an optimized nucleic acid sequence comprising or consisting of SEQ ID NO: 398.

In some embodiments, a PUF110 protein (original sequence) of the disclosure comprises or consists of the amino acid sequence of SEQ ID NO: 399.

In some embodiments, a PUF110 protein of the disclosure is encoded by an optimized nucleic acid sequence comprising or consisting of SEQ ID NO: 400.

Exemplary PUF RNA-binding proteins (targeting 8 Rho nucleotides) used in the compositions and methods disclosed herein are as follows:

In some embodiments, a PUF08 (targeting 8 nucleotides) of the disclosure comprises or consists of the amino acid sequence of SEQ ID NO: 491.

In some embodiments, a PUF08 (targeting 8 nucleotides) of the disclosure is encoded by a nucleic acid sequence comprising or consisting of SEQ ID NO: 492.

In some embodiments, a PUF16 (targeting 8 nucleotides) of the disclosure comprises or consists of the amino acid sequence of SEQ ID NO: 493.

In some embodiments, a PUF16 (targeting 8 nucleotides) of the disclosure is encoded by a nucleic acid sequence comprising or consisting of SEQ ID NO: 494.

In some embodiments, a PUF22 (targeting 8 nucleotides) of the disclosure comprises or consists of the amino acid sequence of SEQ ID NO: 495.

In some embodiments, a PUF22 (targeting 8 nucleotides) of the disclosure is encoded by a nucleic acid sequence comprising or consisting of SEQ ID NO: 496.

In some embodiments, a PUF34 (targeting 8 nucleotides) of the disclosure comprises or consists of the amino acid sequence of SEQ ID NO: 497.

In some embodiments, a PUF34 (targeting 8 nucleotides) of the disclosure is encoded by a nucleic acid sequence comprising or consisting of SEQ ID NO: 498.

In some embodiments, a PUF56 (targeting 8 nucleotides) of the disclosure comprises or consists of the amino acid sequence of SEQ ID NO: 499.

In some embodiments, a PUF56 (targeting 8 nucleotides) of the disclosure is encoded by a nucleic acid sequence comprising or consisting of SEQ ID NO: 500.

In some embodiments, a PUF64 (targeting 8 nucleotides) of the disclosure comprises or consists of the amino acid sequence of SEQ ID NO: 501.

In some embodiments, a PUF64 (targeting 8 nucleotides) of the disclosure is encoded by a nucleic acid sequence comprising or consisting of SEQ ID NO: 502.

In some embodiments, a PUF66 (targeting 8 nucleotides) of the disclosure comprises or consists of the amino acid sequence of SEQ ID NO: 503.

In some embodiments, a PUF66 (targeting 8 nucleotides) of the disclosure is encoded by a nucleic acid sequence comprising or consisting of SEQ ID NO: 504.

In some embodiments, a PUF90 (targeting 8 nucleotides) of the disclosure comprises or consists of the amino acid sequence of SEQ ID NO: 505.

In some embodiments, a PUF90 (targeting 8 nucleotides) of the disclosure is encoded by a nucleic acid sequence comprising or consisting of SEQ ID NO: 506.

In some embodiments, a PUF102 (targeting 8 nucleotides) of the disclosure comprises or consists of the amino acid sequence of SEQ ID NO: 507.

In some embodiments, a PUF102 (targeting 8 nucleotides) of the disclosure is encoded by a nucleic acid sequence comprising or consisting of SEQ ID NO: 508.

In some embodiments, a PUF112 (targeting 8 nucleotides) of the disclosure comprises or consists of the amino acid sequence of SEQ ID NO: 509.

In some embodiments, a PUF112 (targeting 8 nucleotides) of the disclosure is encoded by a nucleic acid sequence comprising or consisting of SEQ ID NO: 510.

In some embodiments, a PUF122 (targeting 8 nucleotides) of the disclosure comprises or consists of the amino acid sequence of SEQ ID NO: 511.

In some embodiments, a PUF122 (targeting 8 nucleotides) of the disclosure is encoded by a nucleic acid sequence comprising or consisting of SEQ ID NO: 512.

In some embodiments, a PUF128 (targeting 8 nucleotides) of the disclosure comprises or consists of the amino acid sequence of SEQ ID NO: 513.

In some embodiments, a PUF128 (targeting 8 nucleotides) of the disclosure is encoded by a nucleic acid sequence comprising or consisting of SEQ ID NO: 514.

In some embodiments, a PUF130 (targeting 8 nucleotides) of the disclosure comprises or consists of the amino acid sequence of SEQ ID NO: 515.

In some embodiments, a PUF130 (targeting 8 nucleotides) of the disclosure is encoded by a nucleic acid sequence comprising or consisting of SEQ ID NO: 516.

In some embodiments, a PUF154 (targeting 8 nucleotides) of the disclosure comprises or consists of the amino acid sequence of SEQ ID NO: 517.

In some embodiments, a PUF154 (targeting 8 nucleotides) of the disclosure is encoded by a nucleic acid sequence comprising or consisting of SEQ ID NO: 518.

In some embodiments, a PUF166 (targeting 8 nucleotides) of the disclosure comprises or consists of the amino acid sequence of SEQ ID NO: 519.

In some embodiments, a PUF166 (targeting 8 nucleotides) of the disclosure is encoded by a nucleic acid sequence comprising or consisting of SEQ ID NO: 520.

Exemplary PUF RNA-binding proteins (targeting 16 Rho nucleotides) are as follows:

In some embodiments, a PUF26 (Design 1-P001IS) of the disclosure comprises or consists of the amino acid sequence of SEQ ID NO: 521.

In some embodiments, a PUF26 (Design 1-P001IS) of the disclosure is encoded by a nucleic acid sequence comprising or consisting of SEQ ID NO: 522.

In some embodiments, a PUF26 (Design 2-P001KZ) of the disclosure comprises or consists of the amino acid sequence of SEQ ID NO: 523.

In some embodiments, a PUF26 (Design 2-P001KZ) of the disclosure is encoded by a nucleic acid sequence comprising or consisting of SEQ ID NO: 524.

In some embodiments, a PUF26 (Design 3-P001LE) of the disclosure comprises or consists of the amino acid sequence of SEQ ID NO: 525.

In some embodiments, a PUF26 (Design 3-P001LE) of the disclosure is encoded by a nucleic acid sequence comprising or consisting of SEQ ID NO: 526.

In some embodiments, a PUF54 (Design 1-P001T) of the disclosure comprises or consists of the amino acid sequence of SEQ ID NO: 527.

In some embodiments, a PUF54 (Design 1-P001T) of the disclosure is encoded by a nucleic acid sequence comprising or consisting of SEQ ID NO: 528.

In some embodiments, a PUF54 (Design 2-P001LA) of the disclosure comprises or consists of the amino acid sequence of SEQ ID NO: 529.

In some embodiments, a PUF54 (Design 2-P001LA) of the disclosure is encoded by a nucleic acid sequence comprising or consisting of SEQ ID NO: 530.

In some embodiments, a PUF54 (Design 3-P001LF) of the disclosure comprises or consists of the amino acid sequence of SEQ ID NO: 531.

In some embodiments, a PUF54 (Design 3-P001LF) of the disclosure is encoded by a nucleic acid sequence comprising or consisting of SEQ ID NO: 532.

In some embodiments, a PUF60 (Design 1-P001IU) of the disclosure comprises or consists of the amino acid sequence of SEQ ID NO: 533.

In some embodiments, a PUF60 (Design 1-P001IU) of the disclosure is encoded by a nucleic acid sequence comprising or consisting of SEQ ID NO: 534.

In some embodiments, a PUF60 (Design 2-P001LB) of the disclosure comprises or consists of the amino acid sequence of SEQ ID NO: 535.

In some embodiments, a PUF60 (Design 2-P001LB) of the disclosure is encoded by a nucleic acid sequence comprising or consisting of SEQ ID NO: 536.

In some embodiments, a PUF60 (Design 3-P001LG) of the disclosure comprises or consists of the amino acid sequence of SEQ ID NO: 537.

In some embodiments, a PUF60 (Design 3-P001LG) of the disclosure is encoded by a nucleic acid sequence comprising or consisting of SEQ ID NO: 538.

In some embodiments, a PUF110 (Design 1-P001IV) of the disclosure comprises or consists of the amino acid sequence of SEQ ID NO: 539.

In some embodiments, a PUF110 (Design 1-P001IV) of the disclosure is encoded by a nucleic acid sequence comprising or consisting of SEQ ID NO: 540.

In some embodiments, a PUF110 (Design 2-P001LC) of the disclosure comprises or consists of the amino acid sequence of SEQ ID NO: 541.

In some embodiments, a PUF110 (Design 2-P001LC) of the disclosure is encoded by a nucleic acid sequence comprising or consisting of SEQ ID NO: 542.

In some embodiments, a PUF110 (Design 3-P001LH) of the disclosure comprises or consists of the amino acid sequence of SEQ ID NO: 543.

In some embodiments, a PUF110 (Design 3-P001LH) of the disclosure is encoded by a nucleic acid sequence comprising or consisting of SEQ ID NO: 545.

Exemplary PUMBY RNA-binding proteins (targeting 8 Rho nucleotides) are as follows:

In some embodiments, a PUM14 protein of the disclosure comprises or consists of the amino acid sequence of SEQ ID NO: 401.

In some embodiments, a PUM14 protein of the disclosure is encoded by a nucleic acid sequence comprising or consisting of SEQ ID NO: 402.

Exemplary PUMBY RNA-binding proteins (targeting 16 Rho nucleotides) are as follows:

In some embodiments, a PUM14 protein (Design 1-P001JG) of the disclosure comprises or consists of the amino acid sequence of SEQ ID NO: 545.

In some embodiments, a PUM14 protein (Design 1-P001JG) of the disclosure is encoded by a nucleic acid sequence comprising or consisting of SEQ ID NO: 546.

In some embodiments, a PUM14 protein (Design 2-P001JB) of the disclosure comprises or consists of the amino acid sequence of SEQ ID NO: 547.

In some embodiments, a PUM14 protein (Design 2-P001JB) of the disclosure is encoded by a nucleic acid sequence comprising or consisting of SEQ ID NO: 548.

In some embodiments of the compositions of the disclosure, at least one of the RNA-binding proteins or RNA-binding portions thereof is a PPR protein. PPR proteins (proteins with pentatricopeptide repeat (PPR) motifs derived from plants) are nuclear-encoded and exclusively controlled at the RNA level organelles (chloroplasts and mitochondria), cutting, translation, splicing, RNA editing, genes specifically acting on RNA stability. PPR proteins are typically a motif of 35 amino acids and have a structure in which a PPR motif is about 10 contiguous amino acids. The combination of PPR motifs can be used for sequence-selective binding to RNA. PPR proteins are often comprised of PPR motifs of about 10 repeat domains. PPR domains or RNA-binding domains may be configured to be catalytically inactive. WO 2013/058404 incorporated herein by reference in its entirety.

In some embodiments, the fusion protein disclosed herein comprises a linker between the at least two RNA-binding polypeptides. In some embodiments, the linker is a peptide linker. In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the at least one RNA-binding protein does not require multimerization for RNA-binding activity. In some embodiments, the at least one RNA-binding protein is not a monomer of a multimer complex. In some embodiments, a multimer protein complex does not comprise the RNA binding protein. In some embodiments, the at least one of RNA-binding protein selectively binds to a target sequence within the RNA molecule. In some embodiments, the at least one RNA-binding protein does not comprise an affinity for a second sequence within the RNA molecule. In some embodiments, the at least one RNA-binding protein does not comprise a high affinity for or selectively bind a second sequence within the RNA molecule. In some embodiments, the at least one RNA-binding protein comprises between 2 and 1300 amino acids, inclusive of the endpoints.

In some embodiments, the at least one RNA-binding protein of the fusion proteins disclosed herein further comprises a sequence encoding a nuclear localization signal (NLS). In some embodiments, a nuclear localization signal (NLS) is positioned at the N-terminus of the RNA binding protein. In some embodiments, the at least one RNA-binding protein comprises an NLS at a C-terminus of the protein. In some embodiments, the at least one RNA-binding protein further comprises a first sequence encoding a first NLS and a second sequence encoding a second NLS. In some embodiments, the first NLS or the second NLS is positioned at the N-terminus of the RNA-binding protein. In some embodiments, the at least one RNA-binding protein comprises the first NLS or the second NLS at a C-terminus of the protein. In some embodiments, the at least one RNA-binding protein further comprises an NES (nuclear export signal) or other peptide tag or secretory signal.

In some embodiments, a fusion protein disclosed herein comprises the at least one RNA-binding protein as a first RNA-binding protein together with a second RNA-binding protein comprising or consisting of a nuclease domain.

In some embodiments, the second RNA-binding polypeptide is operably configured to the first RNA-binding polypeptide at the C-terminus of the first RNA-binding polypeptide. In some embodiments, the second RNA-binding polypeptide is operably configured to the first RNA-binding polypeptide at the N-terminus of the first RNA-binding polypeptide. For example, one such exemplary fusion protein is E99 which is configured so that RNAse1(R39D, N67D, N88A, G89D, R19D, H119N, K41R) is located at the N-terminus of SpyCas9 whereas another exemplary fusion protein, E100, is configured so that RNAse1(R39D, N67D, N88A, G89D, R19D, H119N, K41R) is located at the C-terminus of SpyCas9. In another embodiment, an exemplary fusion protein is a PUF or PUMBY-based first RNA-binding protein fused to a second RNA-binding protein which is an zinc-finger endonuclease known as ZC3H12A of SEQ ID NO: 358 (also termed E17).

### Vectors

In some embodiments of the compositions and methods of the disclosure, a vector comprises a guide RNA of the disclosure. In some embodiments, the vector comprises at least one guide RNA of the disclosure. In some embodiments, the vector comprises one or more guide RNA(s) of the disclosure. In some embodiments, the vector comprises two or more guide RNAs of the disclosure. In one embodiment, the vector comprises three guide RNAs. In one embodiment, the vector comprises four guide RNAs. In some embodiments, the vector further comprises a guided or non-guided RNA-binding protein of the disclosure. In some embodiments, the vector further comprises a RNA-binding fusion protein of the disclosure. In some embodiments, the fusion protein comprises a first RNA binding protein and a second RNA binding protein. In some embodiments, the RNA-guided RNA-binding systems comprising a RNA-binding protein and a gRNA are in a single vector. In a particular embodiment, the single vector comprises the RNA-guided RNA-binding systems which are Cas13d RNA-guided RNA-binding systems. In one embodiment, the single vector comprises the Cas13dRNA-guided RNA-binding systems which are CasRx RNA-guided RNA-binding systems. In another embodiment, the single vector comprises a non-guided RNA-binding system comprising a PUF or PUMBY-based protein fused with a nuclease domain such as ZC3H12A.

In some embodiments of the compositions and methods of the disclosure, a first vector comprises a guide RNA of the disclosure and a second vector comprises an RNA-binding protein or RNA-binding fusion protein of the disclosure. In some embodiments, the first vector comprises at least one guide RNA of the disclosure. In some embodiments, the first vector comprises one or more guide RNA(s) of the disclosure. In some embodiments, the first vector comprises two or more guide RNA(s) of the disclosure. In some embodiments, the fusion protein comprises a first RNA binding protein and a second RNA binding protein. In some embodiments, the first vector and the second vector are identical vectors or vector serotypes. In some embodiments, the first vector and the second vector are not identical vectors or vector serotypes.

In some embodiments of the compositions and methods of the disclosure, the vector is or comprises a component of a “2-component Cas9-based RNA targeting system” comprising (a) nucleic acid sequence encoding an RNA-binding protein or RNA-binding fusion protein and a therapeutic replacement protein of the disclosure; and (b) a single guide RNA (sgRNA) sequence comprising: on its 5′ end, an RNA sequence (or spacer sequence) that hybridizes to or binds to a target RNA sequence (e.g., a pathogenic RNA comprising a target RNA sequence); and on its 3′ end, an RNA sequence (or scaffold sequence) capable of binding to or associating with the CRISPR/Cas9 protein of the fusion protein; and wherein the 2-component RNA targeting system recognizes and alters the target RNA (e.g., comprised within pathogenic target RNA) in a cell in the absence of a PAMmer. In some embodiments, the sequences of the 2-component system are in a single vector. In some embodiments, the spacer sequence of the 2-component system targets RNA comprising one or more gain-or-loss-of-function mutations.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector. Vectors are capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes to which they are operatively-linked. Common expression vectors are often in the form of plasmids. In some embodiments, recombinant expression vectors comprise a nucleic acid provided herein such as e.g., a guide RNA which can be expressed from an RNA sequence or a RNA sequence, and a nucleic acid encoding a Cas 13d protein, in a form suitable for expression of the nucleic acid in a host cell. Recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell. Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., CRISPR transcripts, proteins, enzymes, mutant forms thereof, fusion proteins thereof, etc.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10 (AAVrh10), AAV11 or AAV12. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. In some embodiments, expression control by a promoter is constituitive or ubiquitous. Non-limiting exemplary promoters include a pol III promoter such as, e.g., U6 and H1 promoters and/or a pol II promoter e.g., SV40, CMV (optionally including the CMV enhancer), RSV (Rous Sarcoma Virus LTR promoter (optionally including RSV enhancer), CBA (hybrid CMV enhancer/chicken β-actin), CAG (hybrid CMV enhancer fused to chicken β-actin), truncated CAG, Cbh (hybrid CBA), EF-1a (human longation factor alpha-1) or EFS (short intron-less EF-1 alphs), PGK (phosphoglycerol kinase), CEF (chicken embryo fibroblasts), UBC (ubiquitinC), GUSB (lysosomal enzyme beta-glucuronidase), UCOE (ubiquitous chromatin opening element), hAAT (alpha-1 antitrypsin), TBG (thyroxine binding globulin), Desmin, MCK (muscle creatine kinase), C5-12 (synthetic muscle promoter), NSE (neuron-specific enolase), Synapsin, Synapsin-1 (SYN-1), opsin, PDGF (platelet-derived growth factor), PDGF-A, MecP2 (methyl CpG-binding protein 2), CaMKII (Calcium/Calmodulin-dependent protein kinase II), mGuR2 (metabotropic glutamate receptor 2), NFL (neurofilament light), NFH (neurofilament heavy), nP2, PPE (rat preproenkephalin), ENK (preproenkephalin), Preproenkephalin-neurofilament chimeric promoter, EAAT2 (glutamate transporter), GFAP (glial fibrillary acidic protein), MBP (myelin basic protein), human rhodopsin kinase promoter (hGRK), β-actin promoter, dihydrofolate reductase promoter, and combinations thereof. An “enhancer” is a region of DNA that can be bound by activating proteins to increase the likelihood or frequency of transcription. Non-limiting exemplary enhancers and posttranscriptional regulatory elements include the CMV enhancer, MCK enhancer, R-U5′ segment in LTR of HTLV-1, SV40 enhancer, the intron sequence between exons 2 and 3 of rabbit β-globin, and WPRE.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

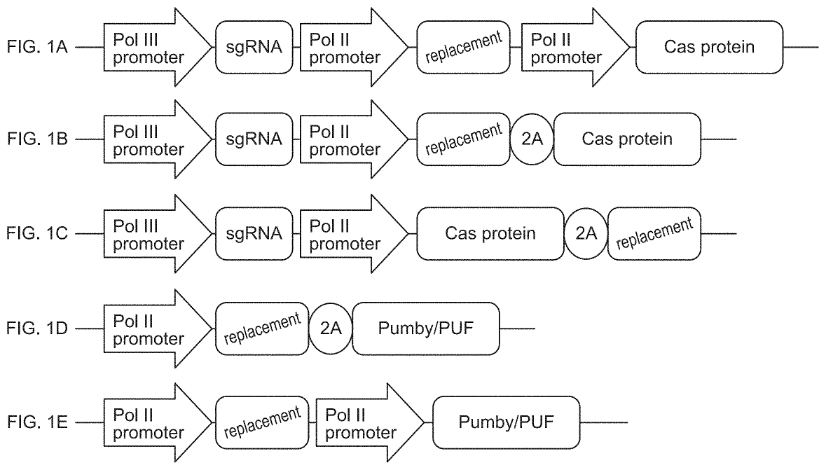

In one embodiment, the vector configuration is shown in e.g., FIGS. 1, 2 or 6. In another embodiment, the vector configuration comprises a promoter or regulatory sequence driving the expression of the nucleic acid encoding the RNA-binding protein in operable linkage with a promoter or regulatory sequence driving the expression of the replacement gene. In another embodiment, a vector configuration comprises an promoter such as a rhodopsin kinase promoter driving expression of the nucleic acid encoding the PUF or PUMBY fusion protein in operable linkage with a promoter such as an opsin promoter driving expression of a nucleic acid sequence encoding the replacement or “hardened” rhodopsin protein. In another embodiment, a vector configuration comprises an promoter such as an opsin promoter driving expression of the nucleic acid encoding the PUF or PUMBY fusion protein in operable linkage with a promoter such as an rhodopsin kinase promoter driving expression of a nucleic acid sequence encoding the replacement or “hardened” rhodopsin protein. In another embodiment, the nucleic acid encoding the RNA-binding protein operably linked to the nucleic acid encoding the replacement protein via an IRES or a 2A peptide.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV2-Tyr mutant vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector, an AAV-Tyr mutant vector, and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Nucleic Acids

Provided herein are the nucleic acid sequences encoding the knockdown and replacement therapeutics disclosed herein for use in gene transfer and expression techniques described herein. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The nucleic acid sequences (e.g., polynucleotide sequences) disclosed herein may be codon-optimized which is a technique well known in the art. In some embodiments disclosed herein, exemplary Cas sequences, such as e.g., a nucleic acid sequence encoding SEQ ID NO: 92 (Cas13d known as CasRx) or the nucleic acid sequence encoding SEQ ID NO: 298 (Cas13d known as CasRx), are codon optimized for expression in human cells. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., a Cas protein, can be generated. In some embodiments, such a sequence is optimized for expression in a host or target cell, such as a host cell used to express the Cas protein or a cell in which the disclosed methods are practiced (such as in a mammalian cell, e.g., a human cell). Codon preferences and codon usage tables for a particular species can be used to engineer isolated nucleic acid molecules encoding a Cas protein (such as one encoding a protein having at least 80%, at least 85%, at least 90%, at least 92%, at least 95%, at least 96%, at least 97%, at least 98%, at least 99%, or 100% sequence identity to its corresponding wild-type protein) that takes advantage of the codon usage preferences of that particular species. For example, the Cas proteins disclosed herein can be designed to have codons that are preferentially used by a particular organism of interest. In one example, an Cas nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule encoding at least one Cas protein (which can be part of a vector) includes at least one Cas protein coding sequence that is codon optimized for expression in a eukaryotic cell, or at least one Cas protein coding sequence codon optimized for expression in a human cell. In one embodiment, such a codon optimized Cas coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a Cas protein having at least 85%, at least 90%, at least 92%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same Cas protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the disclosure, a somatic cell is an ocular cell. An ocular cell includes, without limitation, corneal epithelial cells, keratyocytes, retinal pigment epithelial (RPE) cells, lens epithelial cells, iris pigment epithelial cells, conjunctival fibroblasts, non-pigmented ciliary epithelial cells, trabecular meshwork cells, ocular choroid fibroblasts, conjunctival epithelial cells, In some embodiments, an ocular cell is a retinal cell or a corneal cell. In one embodiment, a retinal cell is a photoreceptor cell or a retinal pigment epithelial cell. In another embodiment, a retinal cell is a ganglion cell, an amacrine cell, a bipolar cell, a horizontal cell, a Müller glial cell, a rod cell, or a cone cell.

In some embodiments of the compositions and methods of the disclosure, a somatic cell of the disclosure is an immune cell. In some embodiments, an immune cell of the disclosure is a lymphocyte. In some embodiments, an immune cell of the disclosure is a T lymphocyte (also referred to herein as a T-cell). Exemplary T-cells of the disclosure include, but are not limited to, naïve T cells, effector T cells, helper T cells, memory T cells, regulatory T cells (Tregs) and Gamma delta T cells. In some embodiments, an immune cell of the disclosure is a B lymphocyte. In some embodiments, an immune cell of the disclosure is a natural killer cell. In some embodiments, an immune cell of the disclosure is an antigen-presenting cell.

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell.

In some embodiments of the compositions and methods of the disclosure, a somatic cell of the disclosure is an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a somatic cell of the disclosure is a neuronal cell. In some embodiments, a neuron cell of the disclosure is a neuron of the central nervous system. In some embodiments, a neuron cell of the disclosure is a neuron of the brain or the spinal cord. In some embodiments, a neuron cell of the disclosure is a neuron of the retina. In some embodiments, a neuron cell of the disclosure is a neuron of a cranial nerve or an optic nerve. In some embodiments, a neuron cell of the disclosure is a neuron of the peripheral nervous system. In some embodiments, a neuron cell of the disclosure is a neuroglial or a glial cell. In some embodiments, a glial of the disclosure is a glial cell of the central nervous system including, but not limited to, oligodendrocytes, astrocytes, ependymal cells, and microglia. In some embodiments, a glial of the disclosure is a glial cell of the peripheral nervous system including, but not limited to, Schwann cells and satellite cells.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods of Use

The disclosure provides a method of modifying level of expression of an RNA molecule of the disclosure or a protein encoded by the RNA molecule comprising contacting the composition of the disclosure and the RNA molecule under conditions suitable for binding of one or more of the guide RNA or the RNA-binding protein or RNA-binding fusion protein (or a portion thereof) to the RNA molecule.

The disclosure provides a method of modifying an activity of a protein encoded by an RNA molecule comprising contacting the composition of the disclosure and the RNA molecule under conditions suitable for binding of one or more of the guide RNA or the RNA-binding protein or the fusion protein (or a portion thereof) to the RNA molecule.

The disclosure provides a method of modifying level of expression of an RNA molecule of the disclosure or a protein encoded by the RNA molecule comprising contacting the composition of the disclosure and a cell comprising the RNA molecule under conditions suitable for binding of one or more of the guide RNA or the RNA-binding protein or fusion protein (or a portion thereof) to the RNA molecule. In some embodiments, the cell is in vivo, in vitro, ex vivo or in situ. In some embodiments, the composition of the disclosure comprises a vector comprising a guide RNA of the disclosure and an RNA-binding protein or fusion protein of the disclosure and the therapeutic replacement protein of the disclosure. In some embodiments, the vector is an AAV.

The disclosure provides a method of modifying an activity of a protein encoded by an RNA molecule comprising contacting the composition of the disclosure and a cell comprising the RNA molecule under conditions suitable for binding of one or more of the guide RNA or the RNA-binding protein or fusion protein (or a portion thereof) to the RNA molecule. In some embodiments, the cell is in vivo, in vitro, ex vivo or in situ. In some embodiments, the composition of the disclosure comprises a vector comprising a guide RNA or a single guide RNA sequence of the disclosure and a nucleic acid sequence encoding the RNA-binding protein or fusion protein of the disclosure and the therapeutic replacement protein of the disclosure. In some embodiments, the vector is an AAV.

The disclosure provides a method of modifying the level of expression of an RNA molecule of the disclosure or a protein encoded by the RNA molecule comprising contacting the composition of the disclosure and the RNA molecule under conditions suitable for RNA nuclease activity wherein the RNA-binding protein or fusion protein induces a break in the RNA molecule.

The disclosure provides a method of modifying an activity of a protein encoded by an RNA molecule comprising contacting the composition of the disclosure and the RNA molecule under conditions suitable for RNA nuclease activity wherein the RNA-binding protein or fusion protein induces a break in the RNA molecule.

The disclosure provides a method of modifying a level of expression of an RNA molecule of the disclosure or a protein encoded by the RNA molecule comprising contacting the composition of the disclosure and a cell comprising the RNA molecule under conditions suitable for RNA nuclease activity wherein the RNA-binding protein or fusion protein induces a break in the RNA molecule. In some embodiments, the composition of the disclosure additionally provides a replacement therapeutic protein which corresponds to a pathogenic RNA comprising a target RNA. In some embodiments, the cell is in vivo, in vitro, ex vivo or in situ. In some embodiments, the composition comprises a vector comprising composition comprising a guide RNA of the disclosure, an RNA-binding fusion protein of the disclosure, and a therapeutic replacement protein of the disclosure. In some embodiments, the vector is an AAV.

The disclosure provides a method of modifying an activity of a protein encoded by an RNA molecule comprising contacting the composition and a cell comprising the RNA molecule under conditions suitable for RNA nuclease activity wherein the RNA-binding protein or fusion protein induces a break in the RNA molecule. In some embodiments, the cell is in vivo, in vitro, ex vivo or in situ. In some embodiments, the composition comprises a vector comprising composition comprising a guide RNA or a single guide RNA of the disclosure and a nucleic acid sequence encoding an RNA-binding protein or fusion protein of the disclosure and a therapeutic replacement protein. In some embodiments, the vector is an AAV.

The disclosure provides a method of treating a disease or disorder comprising administering to a subject a therapeutically effective amount of a composition of the disclosure.

The disclosure provides a method of treating a disease or disorder comprising administering to a subject a therapeutically effective amount of a composition of the disclosure, wherein the composition comprises a vector comprising composition comprising a guide RNA of the disclosure and a nucleic acid sequence encoding an RNA-binding protein or fusion protein of the disclosure and a therapeutic replacement protein of the disclosure, wherein the composition modifies, reduces or ablates a level of expression of a pathogenic target RNA of an RNA molecule of the disclosure or a protein encoded by the RNA molecule (compared to the level of expression of a corresponding wild-type protein), and wherein the therapeutic protein replaces gain-or-loss-of-function mutations encoded by the pathogenic RNA.

The disclosure provides a method of treating a disease or disorder comprising administering to a subject a therapeutically effective amount of a composition of the disclosure, wherein the composition comprises a vector comprising composition comprising a guide RNA of the disclosure and a nucleic acid sequence encoding an RNA-binding protein or fusion protein of the disclosure and a therapeutic replacement protein of the disclosure, wherein the composition modifies, reduces or ablates a level of expression of a pathogenic target RNA of an RNA molecule of the disclosure or a protein encoded by the RNA molecule (compared to the level of expression of a corresponding wild-type protein), and wherein the therapeutic protein replaces gain-or-loss-of-function mutations encoded by the pathogenic RNA.

In some embodiments of the compositions and methods of the disclosure, a disease or disorder includes, without limitation, a disease or disorder related to rhodopsin expression or lack thereof. In some embodiments, the disease or disorder is a retinal degenerative disorder or retinopathy. In some embodiments, the retinal degenerative disorder is retinitis pigmentosa.

Retinitis pigmentosa is an autosomal dominant disorder caused by gain-or-loss-of-function mutations in the rhodopsin gene. Loss of rod photoreceptor cells which express rhodopsin leads to loss of cone photoreceptor cells which causes a degenerative loss of vision. Mutations in the human rhodopsin gene affect the protein's folding, trafficking and activity which most often triggers retinal degeneration in afflicted patients. A single base-substitution at codon position 23 in the human opsin gene (P23H) is also a common cause of retinitis pigmentosa. Retinitis pigmentosa is one of the most common forms of inherited retinal degeneration with a prevalence of 1 in 4000. The disease is the result of varying inheritance patterns (autosomal dominant, autosomal recessive, and X-linked) depending on the mutated gene.

In some embodiments of the compositions and methods of the disclosure, a disease or disorder of the disclosure includes, but is not limited to, a genetic disease or disorder. In some embodiments, the genetic disease or disorder is a single-gene disease or disorder. In some embodiments, the single-gene disease or disorder is an autosomal dominant disease or disorder, an autosomal recessive disease or disorder, an X-chromosome linked (X-linked) disease or disorder, an X-linked dominant disease or disorder, an X-linked recessive disease or disorder, a Y-linked disease or disorder or a mitochondrial disease or disorder. In some embodiments, the genetic disease or disorder is a multiple-gene disease or disorder. In some embodiments, the genetic disease or disorder is a multiple-gene disease or disorder. In some embodiments, the single-gene disease or disorder is an autosomal dominant disease or disorder including, but not limited to, Huntington's disease, neurofibromatosis type 1, neurofibromatosis type 2, Marfan syndrome, hereditary nonpolyposis colorectal cancer, hereditary multiple exostoses, Von Willebrand disease, and acute intermittent porphyria. In some embodiments, the single-gene disease or disorder is an autosomal recessive disease or disorder including, but not limited to, Albinism, Medium-chain acyl-CoA dehydrogenase deficiency, cystic fibrosis, sickle-cell disease, Tay-Sachs disease, Niemann-Pick disease, spinal muscular atrophy, and Roberts syndrome. In some embodiments, the single-gene disease or disorder is X-linked disease or disorder including, but not limited to, muscular dystrophy, Duchenne muscular dystrophy, Hemophilia, Adrenoleukodystrophy (ALD), Rett syndrome, and Hemophilia A. In some embodiments, the single-gene disease or disorder is a mitochondrial disorder including, but not limited to, Leber's hereditary optic neuropathy.

In some embodiments of the compositions and methods of the disclosure, a disease or disorder of the disclosure includes, but is not limited to, an immune disease or disorder. In some embodiments, the immune disease or disorder is an immunodeficiency disease or disorder including, but not limited to, B-cell deficiency, T-cell deficiency, neutropenia, asplenia, complement deficiency, acquired immunodeficiency syndrome (AIDS) and immunodeficiency due to medical intervention (immunosuppression as an intended or adverse effect of a medical therapy). In some embodiments, the immune disease or disorder is an autoimmune disease or disorder including, but not limited to, Achalasia, Addison's disease, Adult Still's disease, Agammaglobulinemia, Alopecia areata, Amyloidosis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, AAT (alpha 1 anti-trypsin deficiency), Wegener's granulomatosis, Wilson disease, Hereditary Hemochromatosis Types 1-5, Type I tyrosinemia, Argininosuccinate Lyase Deficiency, Glycogen storage disease type I-VIII, Citrin deficiency, Cholesteryl ester storage disease, progressive familial intrahepatic cholestasis type 3, polycystic kidney disease, Alstrom syndrome, and Congenital hepatic fibrosis.

In some embodiments of the compositions and methods of the disclosure, a disease or disorder of the disclosure includes, but is not limited to, an inflammatory disease or disorder.

In some embodiments of the compositions and methods of the disclosure, a disease or disorder of the disclosure includes, but is not limited to, a metabolic disease or disorder. In some embodiments, the metabolic disease or disorder is related to inborn errors of the metabolism. In some embodiments, the metabolic disease or disorder related to inborn errors of the metabolism include, without limitation, disorders of amino acid metabolism, disorders of carbohydrate metabolism, disorder or defects of urea cycle, disorders of organic acid metabolism (e.g., organic acidurias), disorders of fatty acid oxidation and mitochondrial metabolism, disorders of porphyrin metabolism, disorders of purine or pyrimidine metabolism, disorders of steroid metabolism, disorders of peroxisomal function, lysosomal storage disorders, and cholestatic diseases.

In some embodiments of the compositions and methods of the disclosure, a disease or disorder of the disclosure includes, but is not limited to, mitochondrial diseases. In some embodiments, the mitochondrial disease includes, but is not limited to, Leber's hereditary optic neuropathy (LHON), Leigh's disease or syndrome, Neuropathy, Ataxia, and Retinitis Pigmentosa (NARP), Kearns-Sayre syndrome (KSS), Pearson syndrome, Chronic Progressive External Opthalmoplegia (CPEO), Mitochondrial neurogastrointestinal encephalopathy syndrome (MNGIE), Mitochondrial Encephalomyopathy Lactic Acidosis and Strokelike Episodes (MELAS), and Mitochondrial Enoyl CoA Reductase Protein Associated Neurodegeneration (MEPAN).

In some embodiments of the compositions and methods of the disclosure, a disease or disorder of the disclosure includes, but is not limited to, a degenerative or a progressive disease or disorder. In some embodiments, the degenerative or a progressive disease or disorder includes, but is not limited to, amyotrophic lateral sclerosis (ALS), Huntington's disease, Alzheimer's disease, and aging.

In some embodiments of the compositions and methods of the disclosure, a disease or disorder of the disclosure includes, but is not limited to, an infectious disease or disorder.

In some embodiments of the compositions and methods of the disclosure, a disease or disorder of the disclosure includes, but is not limited to, a pediatric or a developmental disease or disorder.

In some embodiments of the compositions and methods of the disclosure, a disease or disorder of the disclosure includes, but is not limited to, a cardiovascular disease or disorder.

In some embodiments of the compositions and methods of the disclosure, a disease or disorder of the disclosure includes, but is not limited to, a proliferative disease or disorder. In some embodiments, the proliferative disease or disorder is a cancer. In some embodiments, the cancer includes, but is not limited to, Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Gastrointestinal Carcinoid Tumors, Astrocytomas, Atypical Teratoid/Rhabdoid Tumor, Central Nervous System (Brain Cancer), Basal Cell Carcinoma, Bile Duct Cancer, Bladder Cancer, Bone Cancer, Ewing Sarcoma, Osteosarcoma, Malignant Fibrous Histiocytoma, Brain Tumors, Breast Cancer, Burkitt Lymphoma, Carcinoid Tumor, Carcinoma, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ, Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Parathyroid Cancer, Penile Cancer, Pharyngeal Cancer (Head and Neck Cancer), Pheochromocytoma, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sezary Syndrome, Lymphoma, Skin Cancer, Small Cell Lung Cancer, Small Intestine Cancer, Soft Tissue Sarcoma, Squamous Cell Carcinoma of the Skin, Squamous Neck Cancer, Stomach (Gastric) Cancer, T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Renal Cell Cancer, Urethral Cancer, Uterine Sarcoma, Vaginal Cancer, Vascular Tumors (Soft Tissue Sarcoma), Vulvar Cancer, Wilms Tumor and Other Childhood Kidney Tumors.

In some embodiments of the methods of the disclosure, a subject of the disclosure has been diagnosed with the disease or disorder. In some embodiments, the subject of the disclosure presents at least one sign or symptom of the disease or disorder. In some embodiments, the subject has a biomarker predictive of a risk of developing the disease or disorder. In some embodiments, the biomarker is a genetic mutation.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject systemically. In some embodiments, the composition of the disclosure is administered to the subject by an intravenous route. In some embodiments, the composition of the disclosure is administered to the subject by an injection or an infusion.

In some embodiments of the methods of the disclosure, a composition of the disclosure is administered to the subject locally. In some embodiments, the composition of the disclosure is administered to the subject by an intraosseous, intraocular, intracerebrospinal or intraspinal route. In some embodiments, the composition of the disclosure is administered directly to the cerebral spinal fluid of the central nervous system. In some embodiments, the composition of the disclosure is administered directly to a tissue or fluid of the eye and does not have bioavailability outside of ocular structures. In some embodiments, the composition of the disclosure is administered to the subject by an injection or an infusion.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intrastriatal, subcutaneous, intradermal, intraperitoneal, intratumoral, intravenous, intraocular, and/or parenteral administration. In some embodiments, intraocular administration includes, without limitation, subretinal, intravitreal, deep intravitreal, or topical (via eye drops) administration. In one embodiment, subretinal injection targets photoreceptors and RPE (retinal pigment epithelium) cells. In certain embodiments, the compositions of the present disclosure are formulated for intravenous administration.

### Example Embodiments

Embodiment 1. A composition comprising a nucleic acid sequence encoding an RNA-guided target RNA knockdown and replacement therapeutic comprising (a) an RNA-binding polypeptide or portion thereof, and (b) a therapeutic protein, wherein the RNA-binding polypeptide binds and cleaves a target RNA when guided by a gRNA sequence, wherein a pathogenic RNA comprises the target RNA, and wherein the therapeutic protein is a replacement of gain-or-loss-of-function mutations encoded by the pathogenic RNA.

Or

A composition comprising a nucleic acid sequence encoding a target RNA knockdown and replacement therapeutic comprising (a) an RNA-binding polypeptide or portion thereof, and (b) a therapeutic protein, wherein the RNA-binding polypeptide binds and cleaves a target RNA, wherein a pathogenic RNA comprises the target RNA, and wherein the therapeutic protein is a replacement of gain-or-loss-of-function mutations encoded by the pathogenic RNA.

Or

A composition comprising a nucleic acid sequence encoding a target RNA knockdown and replacement therapeutic comprising (a) an RNA-binding polypeptide or portion thereof, and (b) a therapeutic protein, wherein the RNA-binding polypeptide binds and cleaves a target RNA, wherein a pathogenic RNA comprises the target RNA, and wherein the pathogenic RNA encodes one or more gain-of-function rhodopsin mutations, and wherein the therapeutic protein is wild-type rhodopsin or “hardened” rhodopsin which replaces the gain-or-loss-of-function rhodopsin mutations.

Embodiment 2. The composition of embodiment 1, wherein the therapeutic protein is selected from the group consisting of rhodopsin (Retinitis Pigmentosa), PRPF3 (Retinitis Pigmentosa), PRPF31 (autosomal dominant Retinitis Pigmentosa), GRN (FTD), SOD1 (ALS), PMP22 (Charcot Marie Tooth Disease), PABPN1 (Oculopharangeal Muscular Dystrophy), KCNQ4 (Hearing Loss), CLRN1 (Usher Syndrome), APOE2 (Alzheimer's Disease), APOE4 (Alzheimer's Disease), BEST1 (Eye Disease), MYBPC3 (Familial Cardiomyopathy), TNNT2 (Familial Cardiomyopathy), and TNNI3 (Familial Cardiomyopathy).

Embodiment 3. The composition of embodiment 1 or 2, wherein the pathogenic target sequence comprises or encodes at least one gain-or-loss-of-function mutation.

Embodiment 4. The composition of embodiment 1, wherein the sequence comprising the gRNA comprises a promoter capable of expressing the gRNA in a eukaryotic cell.

Embodiment 5. The composition of embodiment 4, wherein the eukaryotic cell is an animal cell.

Embodiment 6. The composition of embodiment 4, wherein the animal cell is a mammalian cell.

Embodiment 7. The composition of embodiment 5, wherein the animal cell is a human cell.

Embodiment 8. The composition of any one of embodiments 1-7, wherein the promoter is a constitutively active promoter.

Embodiment 9. The composition of any one of embodiments 1-7, wherein the promoter is isolated or derived from a promoter capable of driving expression of an RNA polymerase.

Embodiment 9. The composition of embodiment 9, wherein the promoter is isolated or derived from a U6 promoter.

Embodiment 10. The composition of any one of embodiments 1-9, wherein the promoter is isolated or derived from a promoter capable of driving expression of a transfer RNA (tRNA).

Embodiment 11. The composition of embodiment 10, wherein the promoter is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter.

Embodiment 12. The composition of embodiment 11, wherein the promoter is isolated or derived from a valine tRNA promoter.

Embodiment 13. The composition of any one of embodiments 1-12, wherein the sequence comprising the gRNA comprises a spacer sequence that specifically binds to the target RNA sequence.

Embodiment 14. The composition of embodiment 13, wherein the spacer sequence has at least 50%, 55%, 60%, 65%, 70%, 75%, 80%, 87%, 90%, 95%, 97%, 99% or any percentage in between of complementarity to the target RNA sequence.

Embodiment 15. The composition of embodiment 14, wherein the spacer sequence has 100% complementarity to the target RNA sequence.

Embodiment 16. The composition of any one of embodiments 13-15, wherein the spacer sequence comprises or consists of 20 nucleotides.

Embodiment 17. The composition of any one of embodiments 13-15, wherein the spacer sequence comprises or consists of 26 nucleotides.

Embodiment 18. The composition of any one of embodiments 1-17, wherein the sequence comprising the gRNA comprises a direct repeat (DR) or scaffold sequence that specifically binds to the first RNA binding protein.

Embodiment 20. The composition of embodiment 18, wherein the scaffold sequence comprises a stem-loop structure.

Embodiment 21. The composition of embodiment 19 or 20, wherein the scaffold sequence comprises or consists of 90 nucleotides.

Embodiment 22. The composition of embodiment 19 or 20, wherein the scaffold sequence comprises or consists of 93 nucleotides.

Embodiment 23. The composition of embodiment 22, wherein the scaffold sequence comprises the sequence

(SEQ ID NO: 403)
GUUUAAGAGCUAUGCUGGAAACAGCAUAGCAAGUUUAAAUAAGGCUAG
UCCGUUAUCAACUUGAAAAAGUGGCACCGAGUCGGUGCUUUUUUU.

Embodiment 24. The composition of embodiment 19, wherein the scaffold sequence comprises a step-loop structure.

Embodiment 25. The composition of embodiment 19, wherein the scaffold sequence comprises or consists of 85 nucleotides.

Embodiment 26. The composition of embodiment 25, wherein the scaffold sequence comprises the sequence

(SEQ ID NO: 404)
GGACAGCAUAGCAAGUUAAAAUAAGGCUAGUCCGUUAUCAACUUGAAAAAG
UGGCACCGAGUCGGUGCUUUUU.

Embodiment 27. The composition of embodiment 19, wherein the sequence comprising the gRNA comprises a DR sequence that specifically binds to the first RNA binding protein.

Embodiment 28. The composition of embodiment 27, wherein the DR sequence comprises a stem-loop structure.

Embodiment 29. The composition of embodiment 27, wherein the DR sequence comprises or consists of about 20-36 nucleotides.

Embodiment 30. The composition of embodiment 27, wherein the scaffold sequence comprises or consists of 30-32 nucleotides.

Embodiment 31. The composition of embodiment 27, wherein the DR sequence comprises the nucleotide sequence comprising

(SEQ ID NO: 461)
AACCCCTACCAACTGGTCGGGGTTTGAAAC.

Embodiment 32. The composition of any one of embodiments 1-31, wherein the gRNA does not bind or does not selectively bind to a second sequence within the RNA molecule.

Embodiment 33. The composition of embodiment 32, wherein an RNA genome or an RNA transcriptome comprises the RNA molecule.

Embodiment 34. The composition of any one of embodiments 1-33, wherein the RNA binding protein comprises a CRISPR-Cas protein.

Embodiment 35. The composition of embodiment 34, wherein the CRISPR-Cas protein is a Type II CRISPR-Cas protein.

Embodiment 36. The composition of embodiment 35, wherein the RNA binding protein comprises a Cas9 polypeptide or an RNA-binding portion thereof.

Embodiment 37. The composition of embodiment 34, wherein the CRISPR-Cas protein is a Type V CRISPR-Cas protein.

Embodiment 38. The composition of embodiment 34, wherein the RNA binding protein comprises a Cpf1 polypeptide or an RNA-binding portion thereof.

Embodiment 39. The composition of embodiment 34, wherein the CRISPR-Cas protein is a Type VI CRISPR-Cas protein.

Embodiment 40. The composition of embodiment 39, wherein the RNA binding protein comprises a Cas13 polypeptide or an RNA-binding portion thereof.

Embodiment 41. The composition of any one of embodiments 34-40, wherein the CRISPR-Cas protein comprises a native RNA nuclease activity.

Embodiment 42. The composition of embodiment 41, wherein the native RNA nuclease activity is reduced or inhibited.

Embodiment 43. The composition of embodiment 41, wherein the native RNA nuclease activity is increased or induced.

Embodiment 44. The composition of any one of embodiments 34-43, wherein the CRISPR-Cas protein comprises a native DNA nuclease activity and wherein the native DNA nuclease activity is inhibited, inactive, and/or dead (e.g., dCas).

Embodiment 45. The composition of embodiment 34, wherein the CRISPR-Cas protein comprises a mutation.

Embodiment 46. The composition of embodiment 45, wherein a nuclease domain of the CRISPR-Cas protein comprises the mutation.

Embodiment 47. The composition of embodiment 45, wherein the mutation occurs in a nucleic acid encoding the CRISPR-Cas protein.

Embodiment 48. The composition of embodiment 45, wherein the mutation occurs in an amino acid encoding the CRISPR-Cas protein.

Embodiment 49. The composition of any one of embodiments 45-48, wherein the mutation comprises a substitution, an insertion, a deletion, a frameshift, an inversion, or a transposition.

Embodiment 50. The composition of any one of embodiments 45-49, wherein the mutation comprises a deletion of a nuclease domain, a binding site within the nuclease domain, an active site within the nuclease domain, or at least one essential amino acid residue within the nuclease domain.

Embodiment 51. The composition of any one of embodiments 2-3, wherein the RNA binding protein comprises a Pumilio and FBF (PUF) protein.

Embodiment 52. The composition of embodiment 51, wherein the RNA binding protein comprises a Pumilio-based assembly (PUMBY) protein.

Embodiment 53. The composition of any one of embodiments 51-52, wherein the RNA binding protein does not require multimerization for RNA-binding activity.

Embodiment 54. The composition of embodiment 53, wherein the RNA binding protein is not a monomer of a multimer complex

Embodiment 55. The composition of embodiment 54, wherein a multimer protein complex does not comprise the first RNA binding protein.

Embodiment 56. The composition of any one of embodiments 1-55, wherein the RNA binding protein selectively binds to a pathogenic target sequence within the RNA molecule.

Embodiment 57. The composition of embodiment 56, wherein the RNA binding protein does not comprise an affinity for a second sequence within the RNA molecule.

Embodiment 58. The composition of embodiment 56 or 57, wherein the RNA binding protein does not comprise a high affinity for or selectively bind a second sequence within the RNA molecule.

Embodiment 59. The composition of embodiment 58, wherein an RNA genome or an RNA transcriptome comprises the RNA molecule.

Embodiment 60. The composition of any one of embodiments 1-59, wherein the RNA binding protein comprises between 2 and 1300 amino acids, inclusive of the endpoints.

Embodiment 61. The composition of any one of embodiments 1-60, wherein the sequence encoding the RNA binding protein further comprises a sequence encoding a nuclear localization signal (NLS).

Embodiment 62. The composition of embodiment 61, wherein the sequence encoding a nuclear localization signal (NLS) is positioned 3′ to the sequence encoding the first RNA binding protein.

Embodiment 63. The composition of embodiment 62, wherein the RNA binding protein comprises an NLS at a C-terminus of the protein.

Embodiment 64. The composition of any one of embodiments 1-63, wherein the sequence encoding the RNA binding protein further comprises a first sequence encoding a first NLS and a second sequence encoding a second NLS.

Embodiment 65. The composition of embodiment 64, wherein the sequence encoding the first NLS or the second NLS is positioned 3′ to the sequence encoding the RNA binding protein.

Embodiment 66. The composition of embodiment 65, wherein the RNA binding protein comprises the first NLS or the second NLS at a C-terminus of the protein.

Embodiment 67. The composition of any one of embodiments 1-66, wherein the second RNA binding protein comprises or consists of a nuclease domain.

Embodiment 68. A composition comprising a sequence encoding 1) a target RNA-binding fusion protein comprising (a) a sequence encoding a first RNA-binding polypeptide or portion thereof, and (b) a sequence encoding a second RNA-binding polypeptide, wherein the first RNA-binding polypeptide binds a pathogenic target RNA not guided by a gRNA sequence, and wherein the second RNA-binding polypeptide comprises RNA-nuclease activity; and 2) a therapeutic replacement protein, wherein the therapeutic replacement protein replaces a corresponding gene comprising at least one gain-or-loss-of-function mutation encoded by the pathogenic target RNA.

Embodiment 69. The composition of embodiment 68, wherein the first RNA-binding polypeptide or portion thereof is a PUF, PUMBY, or PPR polypeptide or portion thereof. Embodiment 70. A method for modifying the level of expression of a pathogenic RNA molecule or a protein encoded by the RNA molecule, the method comprising contacting the composition of embodiments 1, 2, 3 or 68 and the RNA molecule under conditions suitable for binding of the RNA-binding protein or a portion thereof to the RNA molecule. Embodiment 71. A method of manufacturing the RNA-targeting knockdown and replacement compositions disclosed herein or the vectors comprising the RNA-targeting knockdown and replacement compositions disclosed herein.

### EXAMPLES

### Example 1: RNA-Guided Cleavage of Target mRNAs

Various RNA-targeting proteins with and without an effector nuclease were constructed. The RNA-targeting proteins are either CRISPR-associated (Cas) proteins or engineered RNA binding proteins known as PUF or Pumby proteins (FIG. 1A-1E). Plasmids encoding the RNA-guided-targeting RNA-binding proteins are co-transfected with a plasmid encoding a corresponding guide RNA that targets a target RNA sequence, e.g., in genes encoding SOD1, human Rhodopsin, PRPF3, PMP22, PABPN1, KCNQ4, CLRN1, APOE2, APOE4, BEST1, MYBPC3, TNNT2, TNN13, or some other gene or mutated gene which causes a disease or leads to a disorder. Plasmids and vectors were designed using exemplary guide RNA spacer sequences which are specific to the target RNA. See SEQ ID NO: 250 to SEQ ID NO: 24960 for exemplary gRNA sequences targeting RHO, SOD1, PMP22, PABPN1, KCNQ4, CLRN1, APOE2, TNNI3, BEST1, MYBPC3, and TNNT2. A plasmid encoding a Cas13d RNA-guided-targeting RNA-binding protein was co-transfected with a plasmid encoding a corresponding guide RNA that targets a target RNA sequence. A Cas13d system based on CasRx sequences was used. Three gRNAs comprising the below spacer sequences targeting rhodopsin target RNA were constructed and used for knockdown of the rhodopsin target sequence below. The gRNAs comprised a CasRx DR sequence with the nucleic acid sequence AACCCCTACCAACTGGTCGGGGTTTGAAAC (SEQ ID NO: 461). The transfected cell line was co-transfected with a plasmid encoding the target RNA. In addition, a cell line which natively expressed the target RNA is used. The level of the target RNA was evaluated by RT-PCR. We observed knockdown of WT RHO containing mRNA.

Spacer sequences and target sequences used for Rho targeting are as detailed in table 2.

TABLE 2
Spacer sequences and target
sequences used for Rho targeting

| Spacer | Spacer Sequences | Target Sequences |
|---|---|---|
| Rho | ACATGTAGATGACAAAAGA | CAACGAGTCTTTTGTCATC |
| guide 1 | CTCGTTG (SEQ ID NO: | TACATGT (SEQ ID NO: |
| | 465) | 462) |
| Rho | TGAAGATGTAGAATGCCAC | CGCCAGCGTGGCATTCTAC |
| guide 2 | GCTGGCG (SEQ ID NO: | ATCTTCA (SEQ ID NO: |
| | 409) | 463) |
| Rho | ACTGCTTGTTCATCATGAT | CATCTATATCATGATGAAC |
| guide 3 | ATAGATG (SEQ ID NO: | AAGCAGT (SEQ ID NO: |
| | 466) | 464) |

### Example 2: Simultaneous Knockdown and Replacement of Target Genes

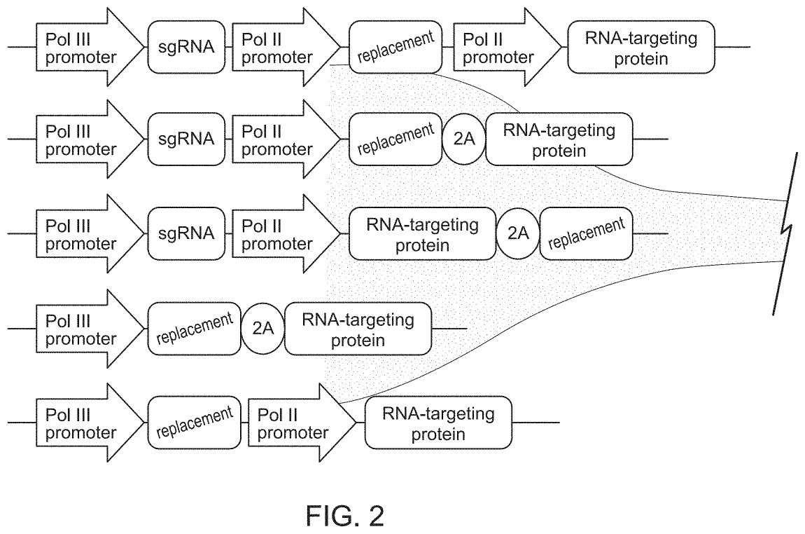

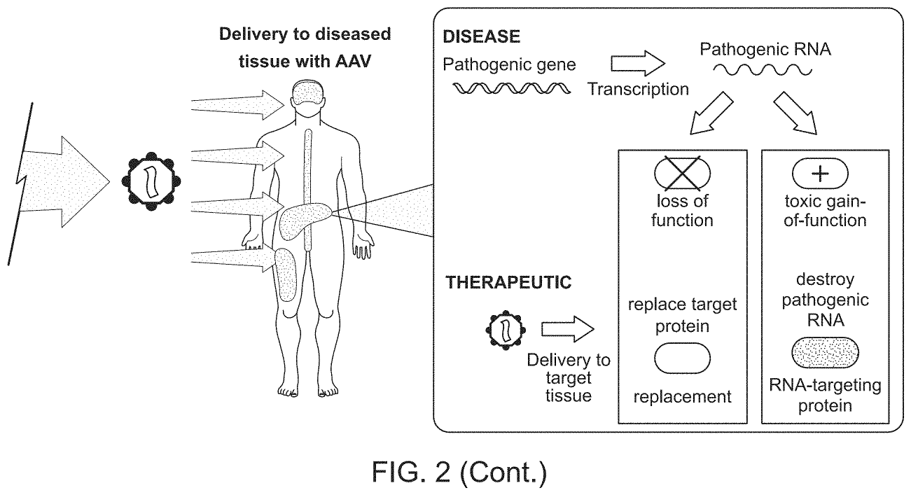

Vectors that carry an RNA-targeting system described in Example 1 with a codon-optimized version of the targeted gene, lacking the corresponding pathogenic mutation, were constructed (FIG. 2). The resulting vectors are capable of knocking down the endogenous, mutated gene and reconstituting expression of the same gene with a wild-type copy. Cells are transfected with the vectors. In addition, cells are infected with AAV vectors comprising the RNA-targeting systems (FIG. 2). We assess levels of both the mutated gene in cells and levels of the reconstituted, therapeutic replacement gene (FIG. 2).

### Example 3: Simultaneous Knockdown and Replacement of Target Genes in a Model of Disease

Vectors that carry an RNA-targeting system described in Example 1 with a codon-optimized version of the targeted gene, lacking the corresponding pathogenic mutation, were constructed. The resulting vectors are capable of knocking down the endogenous, mutated gene and reconstituting expression of the same gene with a wild-type copy. Mice harboring mutated copies of one of the following genes are treated with AAV vectors carrying the above systems (associated human disease in parentheses): rhodopsin (Retinitis Pigmentosa), PRPF3 (Retinitis Pigmentosa), PRPF31 (autosomal dominant Retinitis Pigmentosa), GRN (FTD), SOD1 (ALS), PMP22 (Charcot Marie Tooth Disease), PABPN1 (Oculopharangeal Muscular Dystrophy), KCNQ4 (Hearing Loss), CLRN1 (Usher Syndrome), APOE2 (Alzheimer's Disease), APOE4 (Alzheimer's Disease), BEST1 (Eye Disease), MYBPC3 (Familial Cardiomyopathy), TNNT2 (Familial Cardiomyopathy), and TNNI3 (Familial Cardiomyopathy). We assess levels of both the mutated gene in cells and levels of the reconstituted, unmutated therapeutic replacement gene in the target tissue. We also assess functional/behavioral/physiological changes in situations where these phenomena are modulated by the disease model.

### Example 4: Rhodopsin Knockdown and Replacement

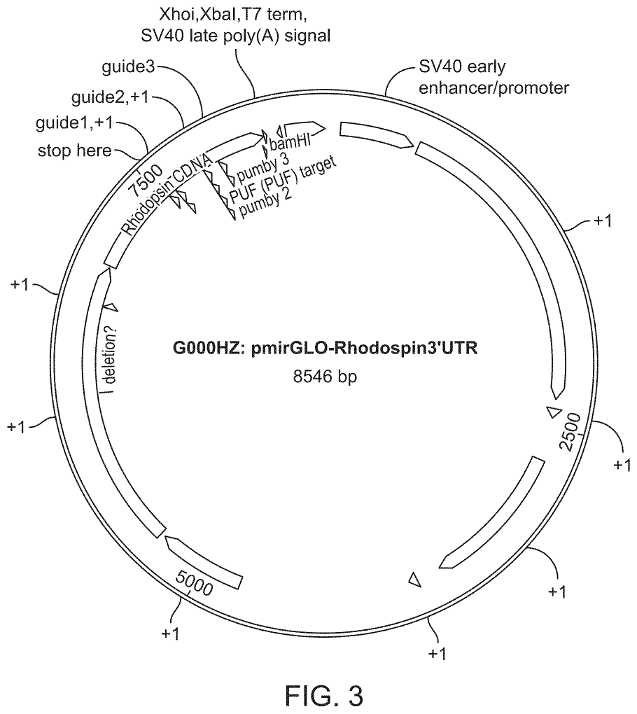

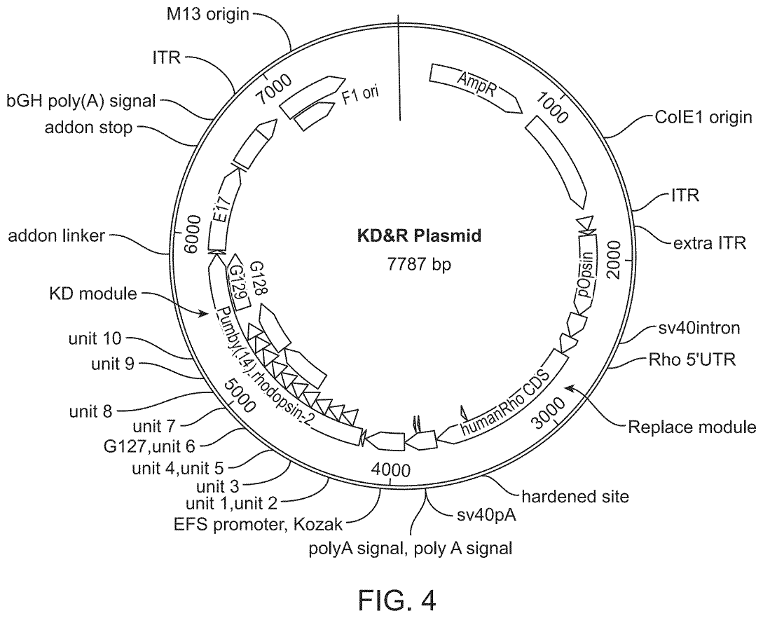

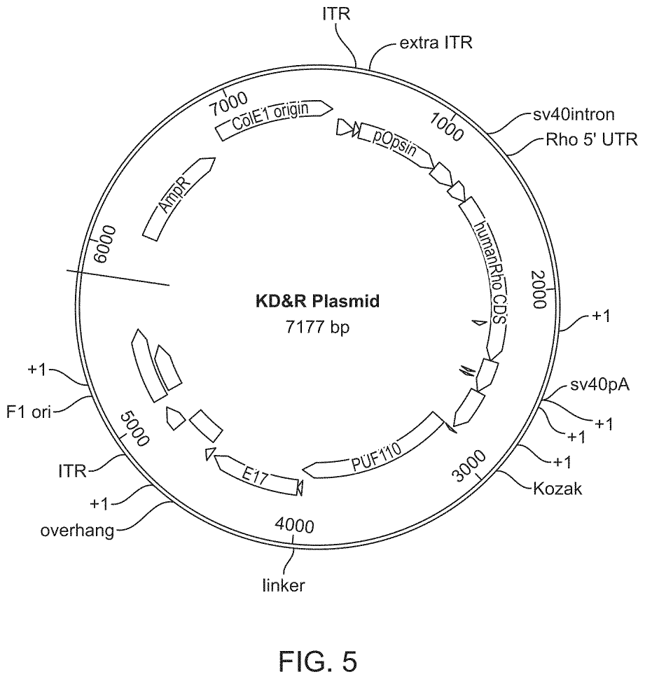

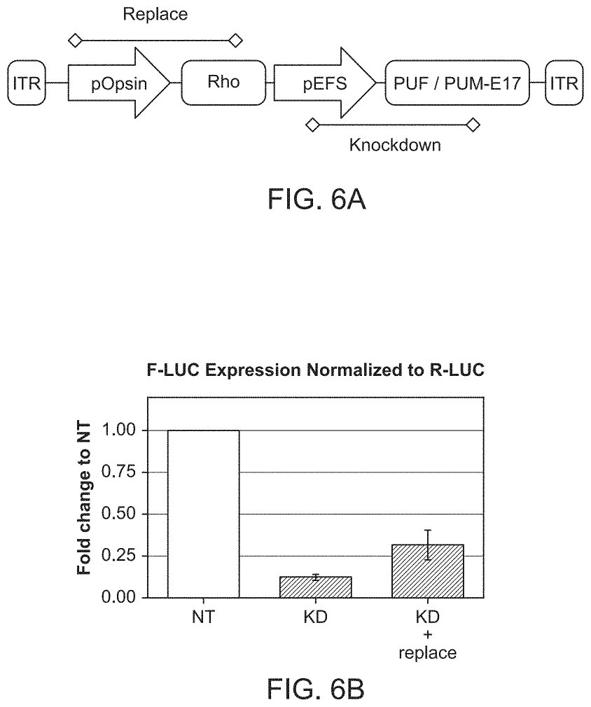

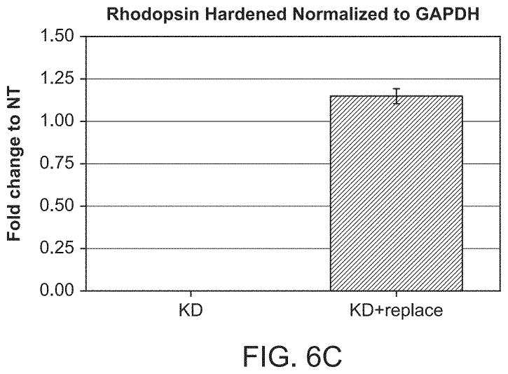

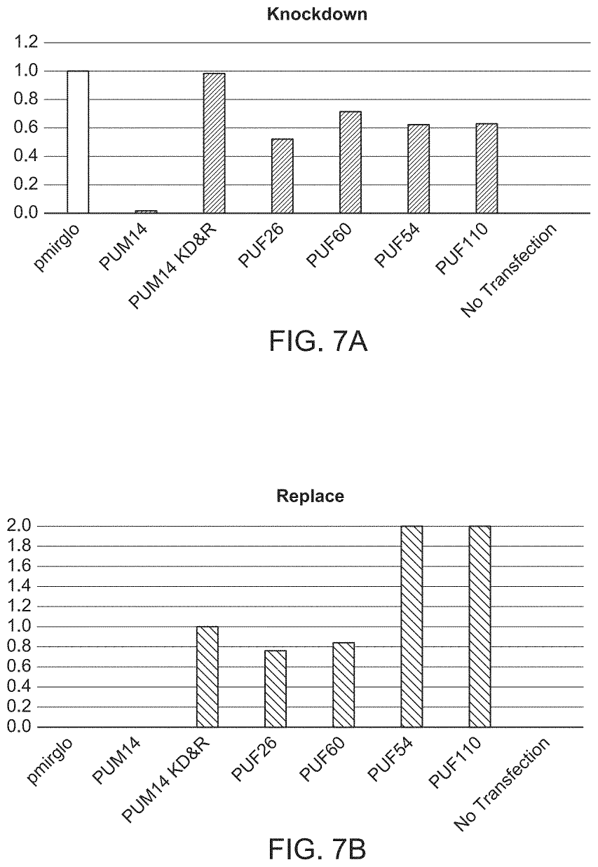

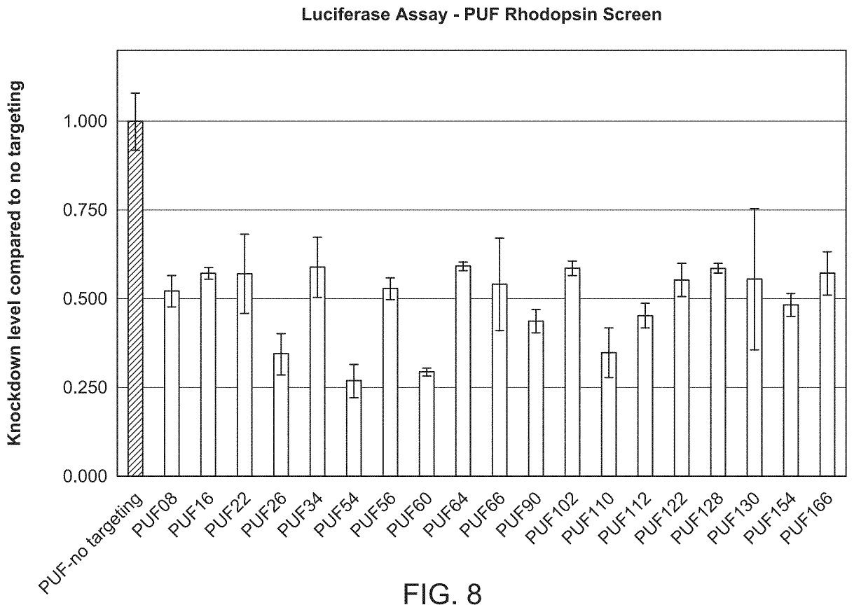

For rhodopsin (RHO) knockdown detection a luciferase reporter assay was designed using the pmirGlo plasmid (FIG. 3) by introducing the wild type (WT) RHO mRNA sequence in the 3′UTR of Firefly luciferase driven by the human phosphoglycerate kinase (hPGK). The reporter plasmid also expressed Renilla luciferase driven by the SV40 promoter for normalization purposes. For knockdown and replacement of RHO 500 ng of the ‘Knockdown and Replace’ PUM and PUF constructs (1 PUMBY construct PUM14, 4 PUF constructs 26, 54, 60, 110 with different optimized PUF sequences—PUF sequences listed below) that express “hardened” Rhodopsin (RHO) open reading frame driven by the opsin promoter and EFS-promoter driven PUMBY or PUF protein linked to ZC3H12A, also termed E17 (FIG. 4, FIG. 5, FIG. 6A) targeting, for cleavage, a specific site on the WT RHO mRNA were transfected using Lipofectamine 3000 (Thermo) into CosM6 cells (according to the manufacturer's protocol) along with the 100 ng of the pmirGlo reporter. Cells were washed and RNA was collected using the Qiagen RNeasy kit. RT-qPCR for normal and hardened Rhodopsin was performed using the Quantabio 1-step RT-qPCR kit, Biorad qPCR machine and the following primer sets: Firefly Luciferase-Forward: GTGGTGTGCAGCGAGAATAG (SEQ ID NO: 410) Reverse: CGCTCGTTGTAGATGTCGTTAG (SEQ ID NO: 411); Renilla Luciferase-Forward: TTCTGGATTCATCGACTGTG (SEQ ID NO: 412) Reverse: TTCAGCAATATCACGGGTAG (SEQ ID NO: 413); Hardened RHO-Forward: ACTGCATGCTCACCACCAT (SEQ ID NO: 414) Reverse: CGAAGAACTCCAGCATGAGA (SEQ ID NO: 415). Firefly luciferase expression was used as the measure of WT RHO mRNA knockdown normalized Renilla Luciferase mRNA expression used to control for transfection. Hardened Rhodopsin expression was normalized to GAPDH and was a measure of replacement. We observed that our knockdown and replace vectors were able to knockdown WT RHO containing mRNA and decrease Firefly Luciferase expression while simultaneously expressing hardened RHO levels of which were sustained. (FIGS. 6B-C and 7A-B).

TABLE 3
PUF and PUMBY Sequences used in the
Knockdown and Replacement Studies

| | Target sequence | Target sequence | Hardened Sequence |
|---|---|---|---|
| Construct | 8 nucleotides | 16 nucleotides | on replacement |
| PUF110 | UCAUCAUG (SEQ ID | GUCAUCAUCAUGGUC | GTGATTATTATGGTG |
| (A000YH) | NO: 549) | A (SEQ ID NO: 550) | A (SEQ ID NO: 551) |
| PUF54 | CCUGUGGU (SEQ ID | UUGCCCUGUGGUCCU | TCGCTCTCTGGTCTTT |
| (A000XL) | NO: 552) | U (SEQ ID NO: 553) | (SEQ ID NO: 554) |
| PUF60 | GGUGUGUA (SEQ | UGGUGGUGUGUAAGC | TCGTCGTCTGCAAAC |
| (A000XM) | ID NO: 555) | C (SEQ ID NO: 556) | C (SEQ ID NO: 557) |
| PUF26 | UCUACGUC (SEQ ID | ACGCUCUACGUCACC | ACCCTGTATGTGACA |
| (A000XK) | NO: 558) | G (SEQ ID NO: 559) | G (SEQ ID NO: 560) |
| PUMBY14 | GUGGCAUUCUACA | CGUGGCAUUCUACAU | CGTAGCTTTTTATAT |
| (A000FS) | U (SEQ ID NO: 561) | C (SEQ ID NO: 562) | T (SEQ ID NO: 563) |

The following sequences are present at the Knockdown module for the above referenced plasmids.

Original PUF26 amino acid sequence:

(SEQ ID NO: 393
MGRSRLLEDFRNNRYPNLQLREIAGHIMEFSQDQHGSRFIRLKLERATPAE
RQLVFNEILQAAYQLMVDVFGNYVIQKFFEFGSLEQKLALAERIRGHVLSL
ALQMYGSRVIEKALEFIPSDQQNEMVRELDGHVLKCVKDQNGSYVVRKCIE
CVQPQSLQFIIDAFKGQVFALSTHPYGCRVIQRILEHCLPDQTLPILEELH
QHTEQLVQDQYGNYVIQHVLEHGRPEDKSKIVAEIRGNVLVLSQHKFASYV
VRKCVTHASRTERAVLIDEVCTMNDGPHSALYTMMKDQYANYVVQKMIDVA
EPGQRKIVMHKIRPHIATLRKYTYGKHILAKLEKYYMKNGVDLG

The Optimized (for Homo sapiens(Human)) sequence of PUF26

A 285 T 205 C 286 G 292 |GC %: 54.12%|Length: 1068

(SEQ ID NO: 394)
ATGGGAAGGAGCAGACTCCTCGAGGACTTTAGGAACAATAGATACCCCAAC
CTCCAGCTGAGAGAAATCGCCGGCCACATCATGGAGTTCAGCCAAGACCAG
CACGGATCTAGATTCATTAGGCTGAAGCTCGAGAGAGCTACACCCGCCGAG
AGGCAACTGGTGTTCAATGAGATTCTGCAAGCCGCCTACCAGCTCATGGTC
GACGTCTTCGGAAACTACGTGATCCAGAAGTTCTTCGAGTTCGGATCTCTG
GAGCAGAAACTCGCTCTGGCTGAGAGGATCAGAGGCCATGTGCTGTCTCTG
GCTCTCCAGATGTACGGCTCTAGAGTGATCGAGAAAGCCCTCGAGTTCATC
CCCTCCGACCAACAGAATGAGATGGTGAGGGAGCTGGACGGCCACGTGCTG
AAATGTGTGAAGGACCAGAACGGCTCCTACGTCGTGAGAAAGTGCATTGAG
TGCGTGCAGCCCCAGAGCCTCCAGTTTATCATCGACGCCTTCAAGGGCCAA
GTGTTCGCTCTCAGCACCCATCCTTACGGCTGTAGAGTCATCCAGAGAATT
CTGGAGCATTGCCTCCCCGACCAGACACTGCCTATTCTCGAGGAGCTCCAT
CAGCATACCGAGCAACTCGTCCAAGACCAGTACGGCAACTACGTGATTCAG
CATGTGCTGGAGCATGGCAGACCCGAGGACAAGAGCAAGATCGTGGCTGAG
ATCAGAGGCAATGTGCTGGTGCTGAGCCAGCACAAATTCGCCAGCTATGTG
GTGAGGAAGTGTGTGACACACGCCTCTAGAACAGAGAGGGCTGTGCTCATC
GATGAGGTGTGCACCATGAACGATGGCCCTCACAGCGCTCTGTACACCATG
ATGAAGGACCAGTACGCCAACTACGTGGTGCAGAAAATGATCGACGTGGCT
GAGCCCGGCCAGAGGAAAATCGTGATGCACAAGATCAGACCTCATATCGCC
ACCCTCAGAAAGTACACCTATGGCAAACACATTCTGGCCAAGCTCGAGAAG
TACTACATGAAAAATGGCGTCGATCTGGGC

The original sequence of PUF54

(SEQ ID NO: 395)
MGRSRLLEDFRNNRYPNLQLREIAGHIMEFSQDQHGNRFIQLKLERATPAE
RQLVFNEILQAAYQLMVDVFGSYVIEKFFEFGSLEQKLALAERIRGHVLSL
ALQMYGSRVIEKALEFIPSDQQNEMVRELDGHVLKCVKDQNGNYVVQKCIE
CVQPQSLQFIIDAFKGQVFALSTHPYGSRVIERILEHCLPDQTLPILEELH
QHTEQLVQDQYGNYVIQHVLEHGRPEDKSKIVAEIRGNVLVLSQHKFASYV
VRKCVTHASRTERAVLIDEVCTMNDGPHSALYTMMKDQYASYVVRKMIDVA
EPGQRKIVMHKIRPHIATLRKYTYGKHILAKLEKYYMKNGVDLG

The Optimized (for Homo sapiens(Human)) sequence of PUF54

A 290 T 194 C 285 G 299 |GC %: 54.68%|Length: 1068

(SEQ ID NO: 396)
ATGGGAAGATCCAGACTGCTGGAGGACTTTAGAAACAATAGGTACCCCAAT
CTGCAGCTGAGAGAGATCGCCGGCCACATCATGGAATTCAGCCAAGACCAG
CACGGCAATAGATTCATCCAGCTGAAGCTCGAGAGGGCTACACCCGCTGAG
AGGCAGCTGGTCTTCAACGAGATTCTGCAAGCCGCCTATCAACTGATGGTG
GACGTGTTCGGCAGCTATGTGATCGAGAAGTTCTTCGAATTCGGCTCTCTG
GAACAGAAGCTGGCTCTGGCCGAGAGGATCAGAGGCCATGTGCTGTCTCTG
GCTCTGCAGATGTACGGCTCTAGAGTCATCGAGAAGGCCCTCGAGTTCATC
CCCTCCGACCAACAGAACGAGATGGTGAGGGAGCTGGACGGACACGTGCTG
AAGTGCGTGAAGGACCAGAACGGAAACTACGTCGTCCAGAAGTGCATCGAA
TGCGTGCAGCCCCAGAGCCTCCAGTTCATTATCGACGCCTTCAAGGGCCAA
GTGTTCGCCCTCAGCACACACCCTTACGGAAGCAGAGTGATCGAGAGGATT
CTGGAGCACTGTCTGCCCGACCAGACACTGCCTATTCTGGAGGAGCTGCAC
CAACACACAGAGCAGCTGGTGCAAGACCAGTACGGCAACTATGTCATTCAG
CACGTCCTCGAGCATGGCAGACCCGAGGACAAAAGCAAGATCGTCGCCGAA
ATCAGAGGCAATGTGCTGGTGCTCAGCCAACACAAGTTCGCTTCCTACGTC
GTGAGGAAGTGCGTGACACACGCTTCCAGAACAGAGAGAGCCGTGCTCATC
GATGAGGTGTGCACCATGAACGATGGCCCTCACAGCGCTCTGTATACCATG
ATGAAGGACCAATACGCCAGCTATGTGGTGAGAAAGATGATCGACGTGGCT
GAACCCGGCCAGAGAAAGATCGTGATGCACAAGATCAGACCCCACATTGCC
ACACTGAGGAAGTATACCTACGGCAAGCACATTCTGGCCAAGCTCGAGAAG
TACTACATGAAGAACGGAGTGGATCTGGGC

The original sequence of PUF60

(SEQ ID NO: 397)
MGRSRLLEDFRNNRYPNLQLREIAGHIMEFSQDQHGSRFIQLKLERATPAE
RQLVFNEILQAAYQLMVDVFGNYVIQKFFEFGSLEQKLALAERIRGHVLSL
ALQMYGSRVIEKALEFIPSDQQNEMVRELDGHVLKCVKDQNGNYVVQKCIE
CVQPQSLQFIIDAFKGQVFALSTHPYGSRVIERILEHCLPDQTLPILEELH
QHTEQLVQDQYGNYVIQHVLEHGRPEDKSKIVAEIRGNVLVLSQHKFASNV
VEKCVTHASRTERAVLIDEVCTMNDGPHSALYTMMKDQYASYVVEKMIDVA
EPGQRKIVMHKIRPHIATLRKYTYGKHILAKLEKYYMKNGVDLG

The Optimized (for Homo sapiens(Human)) sequence of PUF60

A 288 T 201 C 281 G 298 |GC %: 54.21%|Length: 1068

(SEQ ID NO: 398)
ATGGGAAGATCCAGACTGCTGGAGGACTTTAGAAATAATAGATACCCCAAT
CTGCAGCTGAGGGAAATCGCTGGCCACATCATGGAGTTCTCCCAAGACCAG
CATGGATCTAGATTCATCCAGCTGAAGCTCGAGAGAGCCACCCCCGCCGAA
AGGCAGCTCGTCTTCAACGAAATTCTGCAAGCCGCCTACCAACTGATGGTG
GATGTGTTTGGCAACTACGTGATCCAGAAGTTCTTCGAATTTGGCAGCCTC
GAGCAGAAGCTGGCTCTGGCCGAAAGAATTAGAGGCCATGTGCTGTCTCTG
GCCCTCCAGATGTATGGCTCTAGAGTCATCGAAAAGGCTCTGGAGTTCATC
CCCTCCGACCAGCAGAACGAGATGGTGAGAGAGCTCGACGGACATGTGCTG
AAGTGTGTGAAGGACCAGAACGGCAATTACGTCGTCCAGAAGTGCATCGAG
TGCGTGCAGCCCCAGTCTCTGCAGTTTATCATCGACGCCTTCAAGGGCCAA
GTGTTCGCTCTGAGCACACACCCTTACGGCAGCAGAGTGATCGAGAGGATT
CTGGAACACTGTCTGCCCGACCAGACACTGCCTATTCTGGAGGAGCTGCAC
CAGCACACAGAGCAGCTGGTGCAAGACCAGTACGGCAACTATGTGATCCAG
CATGTGCTGGAGCATGGCAGACCCGAGGACAAGAGCAAGATCGTGGCCGAA
ATCAGAGGCAACGTGCTGGTGCTGAGCCAGCACAAGTTCGCCTCCAACGTG
GTGGAAAAGTGCGTGACCCACGCTTCTAGAACAGAAAGGGCTGTGCTCATC
GATGAGGTGTGTACCATGAACGATGGCCCTCACAGCGCTCTGTACACCATG
ATGAAAGACCAGTACGCCAGCTACGTGGTGGAGAAAATGATCGACGTCGCT
GAGCCCGGCCAGAGGAAGATCGTGATGCACAAGATCAGACCCCACATTGCC
ACACTGAGGAAGTACACCTATGGCAAACACATTCTGGCCAAGCTCGAGAAG
TACTACATGAAGAACGGAGTGGATCTGGGC

The original sequence of PUF110

(SEQ ID NO: 399)
MGRSRLLEDFRNNRYPNLQLREIAGHIMEFSQDQHGSRFIELKLERATPAE
RQLVFNEILQAAYQLMVDVFGNYVIQKFFEFGSLEQKLALAERIRGHVLSL
ALQMYGCRVIQKALEFIPSDQQNEMVRELDGHVLKCVKDQNGSYVVRKCIE
CVQPQSLQFIIDAFKGQVFALSTHPYGNRVIQRILEHCLPDQTLPILEELH
QHTEQLVQDQYGCYVIQHVLEHGRPEDKSKIVAEIRGNVLVLSQHKFASYV
VRKCVTHASRTERAVLIDEVCTMNDGPHSALYTMMKDQYANYVVQKMIDVA
EPGQRKIVMHKIRPHIATLRKYTYGKHILAKLEKYYMKNGVDLG

The Optimized (for Homo sapiens(Human)) sequence of PUF110

A 292 T 196 C 293 G 287 |GC %: 54.31%|Length: 1068

(SEQ ID NO: 400)
ATGGGAAGATCCAGACTGCTGGAGGACTTTAGAAACAATAGGTACCCCAAC
CTCCAGCTGAGAGAAATCGCCGGCCACATCATGGAGTTCAGCCAAGACCAG
CACGGCTCTAGATTTATTGAGCTGAAGCTCGAGAGAGCCACCCCCGCCGAG
AGGCAACTGGTGTTCAATGAGATTCTGCAAGCCGCCTACCAGCTCATGGTC
GACGTCTTCGGCAACTACGTCATCCAGAAGTTCTTCGAGTTCGGCTCTCTG
GAACAGAAGCTGGCTCTGGCCGAGAGGATCAGAGGCCACGTGCTGTCCCTC
GCTCTGCAGATGTACGGCTGTAGGGTGATCCAGAAGGCTCTGGAGTTCATC
CCTTCCGACCAGCAGAACGAGATGGTGAGAGAGCTGGATGGACACGTGCTG
AAATGCGTCAAGGACCAGAACGGCTCCTATGTGGTGAGAAAGTGCATCGAG
TGCGTGCAGCCCCAGTCTCTGCAGTTCATCATCGACGCCTTCAAGGGCCAA
GTCTTCGCCCTCAGCACACACCCTTACGGAAATAGAGTCATCCAGAGGATT
CTGGAACACTGTCTGCCCGACCAGACACTGCCTATTCTGGAGGAGCTGCAC
CAACACACAGAGCAGCTGGTCCAAGACCAGTATGGCTGCTACGTGATCCAG
CACGTGCTGGAGCATGGAAGACCCGAGGATAAGAGCAAGATCGTCGCCGAA
ATCAGAGGCAATGTGCTGGTGCTCAGCCAACACAAGTTCGCTTCCTACGTC
GTGAGGAAATGCGTGACACACGCTTCTAGAACAGAAAGGGCCGTGCTCATC
GATGAGGTGTGCACCATGAACGATGGCCCCCACAGCGCTCTGTATACCATG
ATGAAGGACCAGTACGCCAACTACGTGGTGCAGAAGATGATCGACGTGGCT
GAGCCCGGCCAGAGGAAGATTGTGATGCACAAGATTAGGCCCCATATCGCC
ACACTGAGAAAGTACACCTACGGAAAGCATATCCTCGCCAAGCTCGAGAAG
TACTACATGAAGAACGGCGTCGACCTCGGC

The PUMBY (PUM14) targeting rhodopsin comprises the amino acid sequence:

(SEQ ID NO: 401)
MGRSRLLEDFRNNRYPNLQLREIAHTEQLVQDQYGNYVIQHVLEHGRPEDK
SKIVAEIRGHTEQLVQDQYGCYVIQHVLEHGRPEDKSKIVAEIRGHTEQLV
QDQYGSYVIRHVLEHGRPEDKSKIVAEIRGHTEQLVQDQYGCYVIQHVLEH
GRPEDKSKIVAEIRGHTEQLVQDQYGNYVIQHVLEHGRPEDKSKIVAEIRG
HTEQLVQDQYGSYVIRHVLEHGRPEDKSKIVAEIRGHTEQLVQDQYGNYVI
QHVLEHGRPEDKSKIVAEIRGHTEQLVQDQYGNYVIQHVLEHGRPEDKSKI
VAEIRGHTEQLVQDQYGCYVIQHVLEHGRPEDKSKIVAEIRGHTEQLVQDQ
YGSYVIRHVLEHGRPEDKSKIVAEIRGHTEQLVQDQYGSYVIEHVLEHGRP
EDKSKIVAEIRGHTEQLVQDQYGSYVIEHVLEHGRPEDKSKIVAEIRGHTE
QLVQDQYGNYVIQHVLEHGRPEDKSKIVAEIRGHTEQLVQDQYGSYVIEHV
LEHGRPEDKSKIVAEIRGPHIATLRKYTYGKHILAKLEKYYMKNGVDLGG
R.

The PUMBY (PUM14) targeting rhodopsin comprises the nucleic acid sequence:

(SEQ ID NO: 402)
ATGGGCAGAAGCCGGCTGCTGGAAGATTTCCGGAACAACAGATACCCCAAC
CTGCAGCTGAGAGAGATCGCCCACACAGAGCAGCTGGTGCAGGACCAGTAC
GGCAACTACGTGATCCAGCATGTGCTGGAACACGGCAGACCCGAGGACAAG
TCTAAGATCGTGGCCGAGATCAGAGGCCACACCGAACAGCTCGTCCAGGAT
CAATACGGCTGTTATGTGATTCAGCACGTCCTCGAGCACGGACGGCCTGAG
GATAAGAGCAAAATTGTGGCCGAAATCCGGGGCCATACTGAACAACTGGTT
CAGGATCAGTATGGGTCCTATGTGATCCGCCACGTCCTGGAACATGGACGC
CCAGAGGACAAAAGCAAGATCGTCGCTGAGATTCGGGGACATACCGAGCAA
CTCGTCCAAGACCAGTACGGCTGTTACGTGATCCAGCATGTGCTGGAACAC
GGCAGACCCGAGGACAAGTCTAAGATCGTGGCCGAGATCAGAGGCCACACC
GAACAGCTGGTGCAGGACCAGTACGGCAACTATGTGATTCAGCACGTCCTC
GAGCACGGACGGCCTGAGGATAAGAGCAAAATTGTGGCCGAAATCCGGGGA
CACACAGAGCAGCTCGTCCAGGATCAGTATGGCTCCTACGTGATCAGACAC
GTTTTGGAGCACGGCAGGCCAGAAGATAAGTCCAAGATTGTCGCTGAGATT
CGCGGGCATACTGAGCAACTGGTGCAAGATCAATACGGGAATTACGTCATC
CAACACGTTCTCGAACATGGAAGGCCAGAGGACAAAAGCAAGATCGTCGCA
GAAATTAGGGGCCATACAGAACAACTGGTCCAGGACCAGTACGGCAACTAC
GTGATCCAGCATGTGCTGGAACACGGCAGACCCGAGGACAAGTCTAAGATC
GTGGCCGAGATCAGAGGCCACACCGAACAGCTGGTGCAGGATCAGTACGGC
TGTTATGTGATTCAGCACGTCCTCGAGCACGGACGGCCTGAGGATAAGAGC
AAAATTGTGGCCGAAATCCGGGGACACACAGAGCAGCTGGTCCAAGACCAG
TATGGAAGCTATGTCATCAGGCACGTCCTGGAACATGGACGCCCAGAGGAC
AAAAGCAAGATCGTCGCTGAGATTCGGGGCCATACTGAGCAGCTCGTTCAG
GACCAATACGGGTCTTACGTGATCGAACACGTGTTGGAGCATGGCAGGCCC
GAAGATAAGTCCAAAATTGTCGCAGAGATACGCGGCCACACCGAACAGCTG
GTGCAGGATCAGTACGGCAGCTACGTGATCGAGCATGTGCTGGAACACGGC
AGACCCGAGGACAAGTCTAAGATCGTGGCCGAGATCAGAGGCCACACCGAG
CAGCTCGTTCAGGACCAGTATGGCAATTATGTGATCCAGCACGTCCTCGAG
CACGGACGGCCTGAGGATAAGAGCAAAATTGTGGCCGAAATCCGGGGACAC
ACAGAGCAACTGGTCCAAGACCAGTACGGCTCCTATGTGATTGAACACGTT
CTGGAACATGGACGCCCAGAGGACAAAAGCAAGATCGTCGCTGAGATTCGG
GGCCCTCACATTGCCACACTGCGGAAGTACACCTACGGCAAGCACATCCTG
GCCAAGCTGGAAAAGTACTACATGAAGAACGGCGTGGACCTCGGCGGCAG
A.

### Example 5: Knockdown Replacement Screening of Additional Candidates

A Rhodopsin (RHO) knockdown detection luciferase reporter assay was described and carried out as in previous Example 4.

Additional PUF candidates were detailed as depicted in Table 4.

TABLE 4
Additional PUF candidates for Knockdown Replacement

| | Target sequence | Target sequence | Hardened Sequence |
|---|---|---|---|
| Construct | 8 nucleotides | 16 nucleotides | on replacement |
| PUF08 | CGGGUGUG | GCGACGGGUGUGGUAC | GCCACCGGCGTCGTGC |
| (P001MC) | (SEQ ID NO: 564) | (SEQ ID NO: 579) | (SEQ ID NO: 594) |
| PUF16 | CAGUUCUC | AUGGCAGUUCUCCAUG | CTGGCAATTTTCTATG |
| (P001MD) | (SEQ ID NO: 565) | (SEQ ID NO: 580) | (SEQ ID NO: 595) |
| PUF22 | CUGGGCUU | CGUGCUGGGCUUCCCC | TGTCCTCGGATTTCCT |
| (P001ME) | (SEQ ID NO: 566) | (SEQ ID NO: 581) | (SEQ ID NO: 596) |
| PUF34 | AACCUAGC | GCUCAACCUAGCCGUG | CCTGAATCTGGCTGTC |
| (P001MG) | (SEQ ID NO: 567) | (SEQ ID NO: 582) | (SEQ ID NO: 597) |
| PUF56 | UGGUCCUG | UUGGUGGUCCUGGCCA | TTAGTCGTGCTCGCTA |
| (P001MI) | (SEQ ID NO: 568) | (SEQ ID NO: 583) | (SEQ ID NO: 598) |
| PUF64 | UUCGGGGA | CCGCUUCGGGGAGAAC | TCGGTTTGGCGAAAAT |
| (P00005) | (SEQ ID NO: 569) | (SEQ ID NO: 584) | (SEQ ID NO: 599) |
| PUF66 | UGCCAUCA | ACCAUGCCAUCAUGGG | ATCACGCTATTATGGG |
| (P001MK) | (SEQ ID NO: 570) | (SEQ ID NO: 585) | (SEQ ID NO: 600) |
| PUF90 | CGUGGUCC | UGUUCGUGGUCCACUU | TGTTTGTCGTGCATTT |
| (P001MM) | (SEQ ID NO: 571) | (SEQ ID NO: 586) | (SEQ ID NO: 601) |
| PUF102 | GCAGCAGG | CCCAGCAGCAGGAGUC | CTCAACAACAAGAATC |
| (P001MN) | (SEQ ID NO: 572) | (SEQ ID NO: 587) | (SEQ ID NO: 602) |
| PUF112 | GCUUUCCU | CAUCGCUUUCCUGAUC | GATTGCATTTCTCATT |
| (P001MP) | (SEQ ID NO: 573) | (SEQ ID NO: 588) | (SEQ ID NO: 603) |
| PUF122 | UCGGUCCC | AACUUCGGUCCCAUCU | AATTTTGGCCCTATTT |
| (P001MQ) | (SEQ ID NO: 574) | (SEQ ID NO: 589) | (SEQ ID NO: 604) |
| PUF128 | GCGCCGCC | AAGAGCGCCGCCAUCU | AAAAGTGCTGCTATTT |
| (P001MR) | (SEQ ID NO: 575) | (SEQ ID NO: 590) | (SEQ ID NO: 605) |
| PUF130 | AACCCUGU | CUACAACCCUGUCAUC | TTATAATCCAGTGATT |
| (P00006) | (SEQ ID NO: 576) | (SEQ ID NO: 591) | (SEQ ID NO: 606) |
| PUF154 | ACUAUAGG | GCCGACUAUAGGCGUC | GCAGATTAGAGCCGAC |
| (P001MS) | (SEQ ID NO: 577) | (SEQ ID NO: 592) | (SEQ ID NO: 607) |
| PUF166 | CACAUAGG | AAGUCACAUAGGCUCC | AACTCTCAGAGCCTCT |
| (P001MT) | (SEQ ID NO: 578) | (SEQ ID NO: 593) | (SEQ ID NO: 608) |

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodimented herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

1. (canceled)

2. A composition comprising a nucleic acid sequence encoding a target RNA knockdown and replacement therapeutic comprising:
(a) a first nucleic acid sequence encoding an RNA-binding polypeptide or portion thereof; and
(b) a second nucleic acid sequence encoding a wild-type rhodopsin therapeutic protein,
wherein the RNA-binding polypeptide binds and cleaves a target rhodopsin RNA and wherein the target rhodopsin RNA encodes a pathogenic rhodopsin protein with one or more gain-or-loss-of-function mutations.

3.-4. (canceled)

5. The composition of claim 2, wherein the target rhodopsin and therapeutic rhodopsin are human rhodopsin.

6. The composition of claim 2, wherein the therapeutic rhodopsin is a hardened rhodopsin.

7. The composition of claim 2, wherein the RNA binding protein comprises a Pumilio and FBF (PUF) protein.

8. The composition of claim 2, wherein the RNA binding protein comprises a Pumilio-based assembly (PUMBY) protein.

9. The composition of claim 2, wherein the target rhodopsin RNA sequence comprises CAACGAGTCTTTTGTCATCTACATGT (SEQ ID NO: 462), CGCCAGCGTGGCATTCTACATCTTCA (SEQ ID NO: 463), or CATCTATATCATGATGAACAAGCAGT (SEQ ID NO: 464).

10. The composition of claim 9, wherein the target rhodopsin RNA encodes an amino acid sequence comprising YASVAFYIFT (SEQ ID NO: 486) at position 268 to 277.

11. The composition of claim 6, wherein the hardened rhodopsin is encoded by a nucleic acid sequence which does not comprise the target rhodopsin RNA comprising GCCAGCGTGGCATTCTACATCTTC (SEQ ID NO: 406).

12. The composition of claim 11, wherein the hardened rhodopsin is encoded by a nucleic acid sequence comprising GCTTCCGTAGCTTTTTATATTTTT (SEQ ID NO: 408).

13. The composition of claim 2, wherein the nucleic acid sequence comprises at least one promoter.

14. The composition of claim 13, wherein the at least one promoter is a constitutive promoter or a tissue-specific promoter.

15. The composition of claim 14, wherein the at least one promoter is selected from the group consisting of opsin promoter, EFS promoter, and both.

16. The composition of claim 2, wherein the nucleic acid sequence comprises two promoters.

17. A vector comprising the composition of claim 2.

18. The vector of claim 17, wherein the vector is selected from the group consisting of: adeno-associated virus, retrovirus, lentivirus, adenovirus, nanoparticle, micelle, liposome, lipoplex, polymersome, polyplex, and dendrimer.

19. A cell comprising the vector of claim 17.

20. The composition of claim 2, wherein the RNA-binding polypeptide is a first RNA-binding polypeptide, and wherein the nucleic acid sequence encodes a second RNA-binding polypeptide which binds RNA in a manner in which it associates with RNA.

21. The composition of claim 20, wherein the second RNA-binding polypeptide associates with RNA in a manner in which it cleaves RNA.

22. The composition of claim 20, wherein the second RNA-binding polypeptide is selected from the group consisting of: RNAse1, RNAse4, RNAse6, RNAse7, RNAse8, RNAse2, RNAse6PL, RNAseL, RNAseT2, RNAse11, RNAseT2-like, NOB1, ENDOV, ENDOG, ENDOD1, hFEN1, hSLFN14, hLACTB2, APEX2, ANG, HRSP12, ZC3H12A, RIDA, PDL6, NTHL, KIAA0391, APEX1, AGO2, EXOG, ZC3H12D, ERN2, PELO, YBEY, CPSF4L, hCG_2002731, ERCC1, RAC1, RAA1, RAB1, DNA2, FLJ35220, FLJ13173, ERCC4, Rnase1(K41R), Rnase1(K41R, D121E), Rnase1(K41R, D121E, H119N), Rnase1(H119N), Rnase1(R39D, N67D, N88A, G89D, R91D, H119N), Rnase1(R39D, N67D, N88A, G89D, R91D, H119N, K41R, D121E), Rnase1(R39D, N67D, N88A, G89D, R91D), TENM1, TENM2, RNAseK, TALEN, ZNF638, and hSMG6.

23. The composition of claim 22, wherein the second RNA-binding polypeptide is ZC3H12A.

24. A method for reducing the level of expression of a pathogenic target RNA molecule or a protein encoded by the pathogenic RNA molecule and replacing gain-or-loss-of-function mutations caused by the pathogenic target RNA with a therapeutic replacement protein, the method comprising contacting the composition of claim 2 and the pathogenic target RNA molecule comprising a target RNA sequence under conditions suitable for binding of the RNA binding protein to the target RNA sequence, wherein the level of expression of the pathogenic target RNA is reduced, and wherein the expression of the pathogenic target RNA is replaced with expression of a therapeutic replacement protein.

25. An adeno-associated viral (AAV) vector comprising the composition of claim 2.
